(19) United States
(12) Patent Application Publication
(10) Pub. No.: US 2003/0114362 A1
(43) Pub. Date: Jun. 19, 2003

(54) Penta-or tetrapeptide binding to somatostatin receptors and the use of the same

(75) Inventors: Sibylle Gruner, Wasserburg (DE); Gyorgy Keri, Budapest (HU); Horst Kessler, Garching (DE); Erika Pinter, Pecs (HU); Richard Schwab, Budapest (HU); Janos Szolcsanyi, Pecs (HU); Aniko Venetianer, Szeged (HU)

Correspondence Address: SCULLY, SCOTT, MURPHY & PRESSER, 400 Garden City Plaza, Garden City, NY, 11530, US

(73) Assignee: Novaspin Biotech GmbH, Garching (DE)

(21) Appl. No.: 10/166,222

(22) Filed: Jun. 7, 2002

(30) Foreign Application Priority Data
Jun. 8, 2001 (DE) 101 27 747.4
Sep. 25, 2001 (DE) 101 47 056.8

(51) Int. Cl. A61K 38/14; A61K 38/12; A61K 38/16; C07K 9/00; C07K 7/64; C07K 7/08; C07K 7/06

(52) U.S. Cl. 514/8; 514/9; 514/12; 530/317; 530/322; 530/324

(57) ABSTRACT

The subject matter of the present invention is a cyclic or linear tetra- or pentapeptide binding to somatostatin receptors. The compounds of the invention are characterised in that they contain the radical of an amino carboxylic acid bearing a five-membered ring in the peptide backbone which may optionally contain O, S, Se, N, or P. These compounds are easy to prepare and display increased stability against peptidases.

The compounds of the present invention induce apoptosis of tumour cells and the use of said compounds for cancer therapy is described. In particular, the compounds are characterised in that they are active even against tumour cells displaying resistance against other somatostatin derivatives such as octreotide. In addition, the use of the compounds of the invention for tumour diagnosis by means of positron-emission tomography is described, as well as their use as agents against neurogenic inflammation.

### PRIOR ART

Programmed cell death, so-called apoptosis, is an important instrument of the organism to prevent or combat cancer. Cells that have suffered an irreparable damage to their DNA express the tumour suppressor protein p53 which induces cell apoptosis. About 50% of all human cancers are characterised by a mutation of p53 which saves the tumour cells from apoptosis.

Somatostatin is a cyclic peptide hormone which holds a key position in several regulatory metabolic processes. At present, five somatostatin receptors, SSTR1 to SSTR5, are known which may be allocated to the class of G-protein coupled receptors. By binding to these receptors, somatostatin, among other things, influences the adenyl cyclase activity, tyrosine phosphatase activity, MAP kinase activity, the regulation of K⁺ channels, Ca²⁺ channels and the activity of different phospholipases.

Somatostatin receptors, especially SSTR1-SSTR3, were also found on various tumour cell lines. For example, tumour cell lines of the pituitary gland (AtT-20), breast cancer cell lines (MCF7) and Langerhans tumour cell lines (Rin m5f, HIT) may be mentioned. Most human tumours also bear somatostatin receptors, usually in several isoforms.

Somatostatin has a very short half-life of just a few minutes in the human body so that it is hardly suitable as a therapeutic agent. Therefore, many efforts have been made to provide somatostatin derivatives that live longer in the human body [Veber et al., Nature 292, 55, 1981; Veber et al., Life Sci. 34, 1371, 1984; Murphy et al., Biochem. Biophys. Res. Commun. 132, 922, 1985; Cai et al., Proc. Natl. Acad. Sci USA 83, 1896, 1986; U.S. Pat. No. 5,480,879].

There are indications already that somatostatin derivatives binding to somatostatin receptors may cause apoptosis of tumour cells. Therefore, influencing apoptosis with somatostatin derivatives is a promising approach for the therapy of cancer.

In fact, several somatostatin derivatives are clinically applied in tumour therapy already. Examples worth mentioning are octreotide, vapreotide and seglitide. It was also possible to demonstrate an antiproliferative effect as well as the induction of apoptosis in some tumour cell lines for the peptide TT-232 [U.S. Pat. No. 5,480,870].

However, the somatostatin derivatives known from the prior art and suitable for inducing apoptosis are characterised by several disadvantages. For example, the peptidic derivatives consisting of natural amino acids (e.g. TT-232) are decomposed by peptidases and therefore have a comparatively short half-life in the body. The development of so-called multipledrug restistances, MDR, against cytostatic agents of tumor cells, poses one of the greatest challenges to modem anti-cancer medicine, since they drastically reduce the possibilities of using these drugs [Diaconu, C.-C.; Szathmári, M.; Kéri, G.; Venetianer, A. Br. J. Cancer 1999, 80, 1197-1203]. Cytostatic agents exhibit pronounced side-effects whereas somatostatin derivatives are generally more easily tolerated.

A lot of the diseases common in the well developed countries are based on inflammatory processes. In all those processes neurogenic inflammation plays an important part.

In all inflammatory processes occurring in the body neurogenic components, such as certain neuropeptides are involved. Neurogenic inflammation consists of a vicious cycle: The inflammation replicates itself, generating chronic inflammation and pain. Neuropeptides released due to inflammation cause yet again inflammation. The exact mechanism of those inflammatory processes is not yet fully understood. However, it is known that neurogenic inflammation is a major cause of many diseases. These include allergic inflammations of mucous membranes and airways, such as asthma, bronchitis, rhinitis and hay fever as well as arthritis, allergic conjunctivitis, urticaria, inflammations of the gastrointestinal system, such as colitis and inflammatory diseases of the skin, such as psoriasis. This list is far from exhaustive.

To date there is no drug on the market, that reliably inhibits neurogenic inflammation, thereby providing a possibility of an efficient treatment of the pathological pictures of the above listed diseases. This results in the misery of chronic pain, which extremely effects the quality of life of these patients. Classic non-steroidal anti-inflammatory drugs like for instance salicylate, amidopyridine, phenylbutazone, flufenamic acid or indomethacin do not inhibit neurogenic inflammation at all. Steroids do inhibit neurogenic inflammation, but only in very high doses, that cause considerable toxic side effects. Opiates alone proved to be effective. However, they cannot be used due to the their tremendous side effects, E. Pinter, J. Szolcsanyi, Neurosci. Lett. 1996, 212, 33-36; J. Szolcsanyi, in Neurog Inflammation (Eds.: P. Geppetti, P. Holzer), CRC, Boca Raton, USA, 1996, pp. 33-42.

Pretreatment with somatostatin prevented experimentally induced neurogenic inflammation. Nonetheless, it is of no therapeutic use due to its extremely short half life (t_{1/2}<1 min) in the body and its lack of selectivity.

CSPANs (peripheral endings of capsaicin-sensitive primary afferent neurons) synthesize and utilize neuropeptides and tachykinins such as substance P(SP) as transmitters. We know that SP plays an important role in the neurogenic inflammatory process, but the exact mechanism is not yet completely understood. However, we do know, that the inhibition of mechanical, chemical and thermal induced SP and CGRP release, prevents the inflammatory process and pain otherwise caused.

Thus, the capsaicin-sensitive peptidergic sensory nerve endings and terminal varicosities (i.e. part of the ending of neurons) equally provide both a nociceptive afferent function as well as an efferent function eliciting a local tissue response. They play an important role in the signaling of neuropathic or inflammatory as well as hot stimulus- or irritant-induced pains; J. Szolcsanyi, in Capsaicin Study Pain (Ed.: J. N. Wood), Publisher: Academic, London, 1993, pp. 1-26

It has been shown that somatostatin can be found in the peripheral endings of capsaicin-sensitive primary afferent neurons (CSPAN) and is liberated upon stimulation. Capsaicin (8-methyl-N-vanillyl-6-nonene amide), the pungent substance of red pepper, selectively stimulates or, in high doses, degenerates a subgroup of primary afferent neurons (small dark nerve cells). Because of this property, this subpopulation of neurons is called “capsaicin-sensitive primary afferent neurons” (CSPAN)[J. Szolcsanyi, R. Porszasz, G. Pethö, in Peripheral neurons in nociception: physio-pharmacological aspects (Eds.: J. M. Besson, G. Gialbaud, I. Ollat), John Libbey, Eurotext, Paris, France, 1994, pp. 109-124; A. Lecci, C. A. Maggi, Regul. Pept. 2001, 101, 1-18; C. A. Maggi, Prog. Neurobiol. (Oxford) 1995, 45, 1-98.]. CSPAN form about one half of the nerve cell population of sensory ganglions. This group includes the C-polymodal nociceptors amounting to about 60 to 70% of C-afferentation of the skin as well as the perivascular chemoceptive interoceptors of the mucous membranes (conjunctiva, airways, urogenital system, etc.) and visceral organs (heart, kidney, stomach etc.), which can be excited by chemical painstimuli (bradykinin, acids, capsaicin). A common property of these nociceptive afferents is that when stimulated, they release tachykinins (TKs) (substance P(SP), neurokinin A), calcitonin gene-related peptide (CGRP) [C. A. Maggi, Prog. Neurobiol. (Oxford) 1995, 45, 1-98] and somatostatin from their peripheral endings. Tachykinins induce plasma extravasation and neurogenic inflammation on the venules whereas CGRP gives rise chiefly to vasodilatation of the arterioles and enhancement of microcirculation [L. A. Chahl, Pharmacol. Ther. 1988, 37, 275-300]. Thus the capsaicin-sensitive peptidergic sensory nerve endings and terminal varicosities equally provide both a nociceptive afferent function as well as an efferent function eliciting a local tissue response. They play an important role in the signalling of neuropathic or inflammatory as well as hot stimulus-or irritant-induced pains [J. Szolcsanyi, in Capsaicin Study Pain (Ed.: J. N. Wood), Publisher: Academic, London, 1993, 1-26].

Therefore, it is an object of the present invention to provide somatostatin derivatives which exhibit an antiproliferative effect on tumor cells, that is, which reduce tumor growth or induce apoptosis, have a longer half-life in the human body than active ingredients known from the prior art and are effective even against tumors which exhibit multiple resistencies against cytostatic agents (multidrug resistance) or are resistant to other somatostatin derivatives such as octreotide.

A further object of the present invention is to provide agents and pharmaceutical compositions, which are useful to inhibit neurogenic and/or non-neurogenic inflammations as well as to alleviate pain. It is another object of the present invention to provide somatostatin derivatives which have the above characteristics and which may be produced in a simple and inexpensive manner at the same time.

Many tumours in glands of the human body are difficult to diagnose. A lot of human tumors bear somatostatin receptors. Therefore, somatostatin derivatives with a sufficient half-life, a suitable pharmacokinetic profile which bind to these receptors or which are internalised by these tumor cells and which bear a radioactive atom should be suitable agents for tumour diagnosis by means of positron-emission tomography.

The so-called SST-receptor scintigraphy is currently the most important clinical method of diagnosis for neuroendocrine tunors [Scarpignato, C.; Pelosini, I. Chemotherapy 2001, 47, 1-29].

It is a further object of the present invention to provide somatostatin derivatives which may be used to diagnose tumours by means of positron-emission tomography.

### SUMMARY OF THE INVENTION

The invention achieves the above object by providing a peptide according to claim 1. Preferred embodiments of the invention are described in the sub-claims 2 to 31.

The different uses of the peptide are specified in claims 32 to 44 and its production in claim 45.

### DESCRIPTION OF THE FIGURES

FIG. 1: Schematic drawing of the functional anatomy of the capsaicin-sensitive primary afferent neuron (CSPAN). Sensory neuropeptides (tachykinins (TKs): —substance P(SP) and neurokinin A (NKA)—and calcitonin gene-related peptide (CGRP)) are synthesized in the perikaryon and transported to both peripheral (1, 2, 3) and central terminals (4) of the CSPANs. Environmental stimuli (mechanical, chemical, thermal) induce the release of sensory neuropeptides (like SP, NKA, and CGRP) from the same nerve terminal at which they activate afferent discharge.

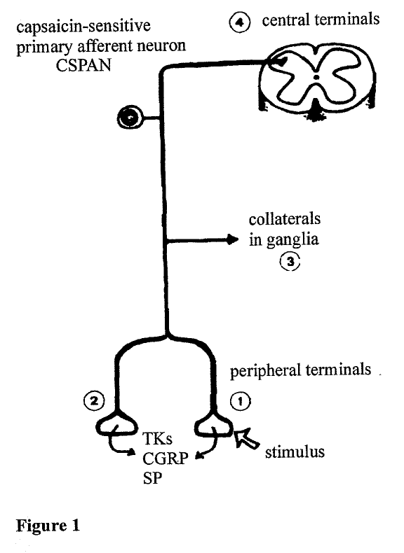

FIG. 2: Illustration of the results obtained in Example 9, showing that Substance P release evoked by electrical stimulation of sensory nerve terminals is inhibited by SGTG, SGA and SGTH in similar extent as elicited by TT-232.

### DETAILED DESCRIPTION OF THE INVENTION

1. Definitions

The following abbreviations are used:

Nomenclature of protected and unprotected natural and unnatural amino acids according to the definition in the Novabiochem Catalogue 2000 under “useful information, nomenclature, abbreviation”, page x et seq. and pages A3-A13.

| | |
|---|---|
| TKs | tachykinins |
| SP | substance P, neuropeptide of the sequence |
| | H-Arg-Pro-Lys-Pro-Gln-Gln-Phe-Phe-Gly-Leu-Met- |
| | NH₂. |
| NKA | neurokinin A |
| CGRP | calcitonin gene-related peptide |
| Bzl | benzyl |
| Bn | benzyl |
| Bip | biphenyl alanine |
| Fmoc-Bip-OH | cas#: [199110-64-0] |
| Bpa | benzophenone alanine |
| Collidine | 2,4,6-trimethyl pyridine |
| DIPEA | diisopropyl ethyl amine |
| DPPA | diphenyl phosphoryl acid |
| equiv | equivalents |
| ESI | electron spray ionisation |
| HATU | [O-(7-azabenzotriazol-1-yl)-1,1,3,3- |
| | tetramethyluronium hexafluorophosphate] |
| HOAt | 1-hydroxy-7-azabenzotriazol |
| ivDde | 1-(4,4-dimethyl-2,6-dioxo-cyclohexylidene)3-methyl |
| | butyl |
| MTT | 3-(4,5-dimethyl-2-thiazolyl)-2,5-diphenyltetrazolium |
| | bromide |
| NMP | N-methyl pyrrolidone |
| ODmab | 4{N-[1-(4,4-dimethyl-2,6-dioxo-cyclohexylidene)-3- |
| | methyl butyl]-amino}benzyloxy |
| TCP-resin: | tritylchloropolystyrene resin |
| TLC: | thin layer chromatography |
| Trt | trityl |
| GABA | 4-aminobutyric acid |
| TEMPO | 2,2,6,6-tetramethylpiperidine-1-oxyl |
| HFIP | hexafluoroisopropanol |
| DCM | dichloromethane |
| HPLC | high performance liquid chromatography |
| XTT | (2,3-bis-(2-methoxy-4-nitro-5-sulfophenyl)-2H- |
| | tetrazolium-5-carboxanilide, disodium salt) |
| Nle | norleucine |
| β-z | 3-amino-3-deoxy-N-9-fluorenylmethoxycarbonyl- |
| | 1,2-isopropylidene-α-D-ribofuranose acid |
| γ-z | 3-amino-3-deoxy-N-9-fluorenylmethoxycarbonyl- |
| | 1,2-isopropylidene-α-D-allofuranose acid |
| Tentagel | trichlorotrityl resin |
| Fmoc | 9-fluorenyloxy carbonyl |
| Boc | t-butyloxycarbonyl |
| Lys | lysine |
| Trp | tryptophan |
| Tyr | tyrosine |
| Tyr(Me) | tyrosine methyl ether |
| Tyr(Bzl) | tyrosine benzyl ether |
| Thr | threonine |
| Thr(Bzl) | threonine benzyl ether |
| Bta | L-3-benzothienyl alanine (L-form: CAS#: |
| | 72120-71-9) |
| | 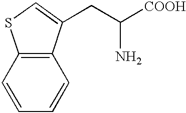 |
| Bip | L-biphenyl alanine (L-Form: CAS#: 155760-02-4) |
| | 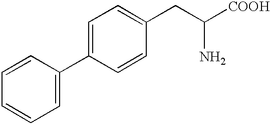 |
| Dip | L-diphenyl alanine (L-Form: CAS#: 1495997-92-2) |
| | 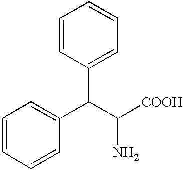 |
| Bpa | 1-benzophenone alanine |
| 1-Nal | 1-naphthyl alanine |
| 2-Nal | 2-naphthyl alanine |
| o-fluoro-Phe | o-fluorophenyl alanine |
| m-fluoro-Phe | m-fluorophenyl alanine |
| p-fluoro-Phe | p-fluorophenyl alanine |
| 2,3-difluoro-Phe | 2,3-difluorophenyl alanine |
| 2,4-difluoro-Phe | 2,4-difluorophenyl alanine |
| 2,5-difluoro-Phe | 2,5-difluorophenyl alanine |
| Phe(F₅) | pentafluorophenyl alanine |
| o-chloro-Phe | o-chlorophenyl alanine |
| m-chloro-Phe | m-chlorophenyl alanine |
| p-chloro-Phe | p-chlorophenyl alanine |
| 2,3-Dichloro-Phe | 2,3-dichlorophenyl alanine |
| 2,4-Dichloro-Phe | 2,4-dichlorophenyl alanine |
| 2,5-Dichloro-Phe | 2,5-dichlorophenyl alanine |
| Phe(Cl₅) | pentachlorophenyl alanine |
| 3-Pal | 3-pyridinyl alanine |
| 4-Pal | 4-pyridinyl alanine |
| Phg | phenyl glycine |
| Thr(Ar) | aryl ether or arylalkyl ether of threonine |
| hPhe | homo-phenyl alanine (L-Form: CAS#: 943-73-7) |
| | 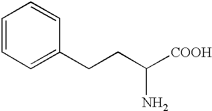 |
| hTyr | homo-tyrosine |
| | 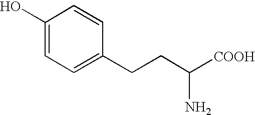 |
| Igl | indanyl glycine |
| Phe(4-NO₂) | 4-nitrophenyl alanine |
| Phe(4-NH-2Clz) | 4-((2-chlorobenzyl)oxycarbonyl-amino)-phenyl |
| | alanine |
| Phe(4-NHz) | 4-(benzyloxycarbonyl-amino) phenyl alanine |
| Pra | propargyl glycine |
| DMF | N,N-dimethyl formamide |
| ESI-MS | Electron Spray Ionisation Mass Spectroscopy |
| MB | Methylene blue |
| MTT | 3-(4,5-dimethyl-2-thiazolyl)-2,5-diphenyltetrazolium |
| | bromide |
| XTT | (2,3-bis-(2-methoxy-4-nitro-5-sulfophenyl)-2H- |
| | tetrazolium-5-carboxanilide, disodium salt) |
| Bpa | 4-benzophenyl alanine |
| Fmoc-Bpa-OH | CAS #11766696-3 |
| Fmoc-D-1-Nal-OH | [138774-93-3] |
| Fmoc-1-Nal-OH | Fmoc-1-naphthyl alanine [96402-49-2] |
| Fmoc-2-Nal-OH | Fmoc-2-naphthyl alanine [136774-94-4] |
| AcOEt | ethyl acetate |
| FC | flash chromatography, chromatography at increased |
| | pressure |
| EDTA | ethylenediiaminetetraacetic acid |
| DFO | desferrioxamine-B |
| DADS | diamidedithiol |

Alkyl within the meaning of the present invention is a branched, unbranched or cyclic alkyl group. Lower alkyl groups having 1 to 10 carbon atoms are preferred; those having 1 to 6 carbon atoms are particularly preferred. Special mention may be made of the radicals methyl, ethyl, propyl, iso-propyl, n-butyl, sec-butyl, tert-butyl, n-pentyl, neo-pentyl, 1-methyl butyl, 2-methyl butyl, 3-methyl butyl, cyclo-pentyl, n-hexyl, 1-methyl pentyl, 2-methyl pentyl, 3-methyl pentyl, 4-methyl pentyl, 1-ethyl butyl, 2-ethyl butyl, 3-ethyl butyl and cyclo-hexyl.

Alkenyl within the meaning of the present invention is a branched, unbranched or cyclic hydrocarbon group comprising one or more unsaturated carbon-carbon bonds. These unsaturated carbon-carbon bonds do not form an aromatic system. Alkenyl groups having 2 to 10 carbon atoms are preferred; those having 2 to 6 carbon atoms are especially preferred. The unsaturated bond may be present at any position within the alkenyl group. Special mention may be made of the radicals ethenyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-methyl ethenyl, 1-methyl-1-propenyl, 1-methyl-2-propenyl, 1-methyl-1-butenyl, 1-methyl-2-butenyl, 1-methyl-3-butenyl, 2-methyl-1-butenyl, 2-methyl-2-butenyl, 2-methyl-3-butenyl, 3-methyl-2-butenyl, 1-methyl-1-pentenyl, 1-methyl-2-pentenyl, 1-methyl-3-pentenyl, 1-methyl-4-pentenyl, 2-methyl-1-pentenyl, 2-methyl-2-pentenyl, 2-methyl-3-pentenyl, 2-methyl-4-pentenyl, 3-methyl-1-pentenyl, 3-methyl-2-pentenyl, 3-methyl-3-pentenyl, 3-methyl-4-pentenyl, 4-methyl-1-pentenyl, 4-methyl-2-pentenyl, 4-methyl-3-pentenyl, 4-methyl-4-pentenyl.

Alkinyl within the meaning of this invention is a branched, unbranched or cyclic hydrocarbon group having one or more di-unsaturated carbon-carbon bonds. Alkinyl groups having 2 to 10 carbon atoms are preferred; those having 2 to 6 carbon atoms are especially preferred. The di-unsaturated bond may be present at any position within the alkinyl group. Special mention may be made of the radicals ethinyl, 1-propinyl, 2-propinyl, 1-butinyl, 2-butinyl, 3-butinyl, 1-pentinyl, 2-pentinyl, 3-pentinyl, 4-pentinyl, 1-hexinyl, 2-hexinyl, 3-hexinyl, 4-hexinyl, 5-hexinyl, 1-methyl-2-propinyl, 1-methyl-2-butinyl, 1-methyl-3-butinyl, 2-methyl-3-butinyl, 3-methyl-1-butinyl, 1-methyl-2-pentinyl, 1-methyl-3-pentinyl, 1-methyl-4-pentinyl, 2-methyl-3-pentinyl, 2-methyl-4-pentinyl, 3-methyl-4-pentinyl, 3-methyl-1-pentinyl, 4-methyl-1-pentinyl und 4-methyl-2-pentinyl.

Aryl within the meaning of this invention is a cyclic aromatic group. The aryl group optionally contains one or more heteroatoms selected from the group consisting of N, S, O so that heteroaryl groups also fall under the term “aryl group” within the meaning of this invention. Aryl groups having 4 to 16 carbon atoms are preferred; benzyl, naphthyl, anthracyl, fluorenyl, pyridyl, pyrazinyl, pyrrolyl, imidazolyl, furanyl, thienyl and indolyl groups are especially preferred.

Arylalkyl within the meaning of the present invention is an aryl group linked to the remainder of the molecule by an alkyl group. The preferred groups listed for this group are also preferred in the present case.

Alkylaryl within the meaning of the present invention is an alkyl group linked to the remainder of the molecule by an aryl group. The preferred groups listed for this group are also preferred in the present case.

Alkoxy within the meaning of the present invention is an alkyl group linked to the remainder of the molecule by an oxygen atom. The preferred groups listed for this group are also preferred in the present case.

Alkenyloxy within the meaning of the present invention is an alkenyl group linked to the remainder of the molecule by an oxygen atom. The preferred groups listed for this group are also preferred in the present case.

Aryloxy within the meaning of the present invention is an aryl group linked to the remainder of the molecule by an oxygen atom. The preferred groups listed for this group are also preferred in the present case.

Arylalkoxy within the meaning of the present invention is an arylalkyl group linked to the remainder of the molecule by an oxygen atom. The preferred groups listed for this group are also preferred in the present case.

Alkylaryloxy within the meaning of the present invention is an alkylaryl group linked to the remainder of the molecule by an oxygen atom. The preferred groups listed for this group are also preferred in the present case.

Thioalkyl within the meaning of the present invention is an alkyl group linked to the remainder of the molecule by a sulfur atom. The preferred groups listed for this group are also preferred in the present case.

Thioalkenyl within the meaning of the present invention is an alkenyl group linked to the remainder of the molecule by a sulfur atom. The preferred groups listed for this group are also preferred in the present case.

Thioaryl within the meaning of the present invention is an aryl group linked to the remainder of the molecule by a sulfur atom. The preferred groups listed for this group are also preferred in the present case.

Selenoalkyl within the meaning of the present invention is an alkyl group linked to the remainder of the molecule by a selenium atom. The preferred groups listed for this group are also preferred in the present case.

Selenoaryl within the meaning of the present invention is an aryl group linked to the remainder of the molecule by a selenium atom. The preferred groups listed for this group are also preferred in the present case.

Alkanoyl within the meaning of the present invention is an alkyl group linked to the remainder of the molecule by a —C(O) group. The preferred groups listed for this group are also preferred in the present case.

Alkenoyl within the meaning of the present invention is an alkenyl group linked to the remainder of the molecule by a —C(O) group. The preferred groups listed for this group are also preferred in the present case.

Alkinoyl within the meaning of the present invention is an alkinyl group linked to the remainder of the molecule by a C(O) group. The preferred groups listed for this group are also preferred in the present case.

Aroyl within the meaning of the present invention is an aryl group linked to the remainder of the molecule by a —C(O) group. The preferred groups listed for this group are also preferred in the present case.

Arylalkanoyl within the meaning of the present invention is an arylalkyl group linked to the remainder of the molecule by a —C(O) group. The preferred groups listed for this group are also preferred in the present case.

Alkylaroyl within the meaning of the present invention is an alkylaryl group linked to the remainder of the molecule by a —C(O) group. The preferred groups listed for this group are also preferred in the present case.

Amidoalkyl within the meaning of the present invention is an alkyl group linked to the remainder of the molecule by an amide linkage. The preferred groups listed for this group are also preferred in the present case.

Amidoalkenyl within the meaning of the present invention is an alkenyl group linked to the remainder of the molecule by an amide linkage. The preferred groups listed for this group are also preferred in the present case.

Amidoalkinyl within the meaning of the present invention is an alkinyl group linked to the remainder of the molecule by an amide group. The preferred groups listed for this group are also preferred in the present case.

Arylalkanoyloxy within the meaning of the present invention is an arylalkyl group linked to the remainder of the molecule by an ester group. The preferred groups listed for this group are also preferred in the present case.

Alkylaroyloxy within the meaning of the present invention is an alkylaryl group linked to the remainder of the molecule by an ester group. The preferred groups listed for this group are also preferred in the present case.

Aminocarboxylic acid within the meaning of the present invention is an α-, β-, or γ-aminocarboxylic acid. Alpha-aminocarboxylic acids occurring in nature are preferred. Unless explicitly defined, all stereo isomers of optically active aminocarboxylic acids are included, especially the D- and L-forms of α-aminocarboxylic acids occurring in nature.

Aliphatic side chains within the meaning of the present invention mean a side chain of an aminocarboxylic acid which is an alkyl group. The side chains of the amino carboxylic acids alanine, valine, leucine, norleucine and isoleucine are preferred. Optionally, the side chain may bear one or more substituents selected from the group consisting of F, Cl, Br, I, alkoxy, alkylthio, alkylseleno.

An aromatic side chain within the meaning of the present invention is a side chain of an aminocarboxylic acid comprising at least one aromatic ring. This ring may be a pure carbocycle or include one or more heteroatoms selected from the group consisting of N, S and O. The aromatic ring may be substituted. It may be linked to the peptide backbone directly or by an alkylene group. Preferred aromatic side chains are the side chains of phenyl alanine, 1- and 2-naphthyl alanine, tyrosine, tryptophan, biphenyl alanine, mono-, di-, tri-, tetra-, and pentahalogenated phenyl alanine, substituted and unsubstituted, especially mono-, di-, tri-, tetra-, and pentahalogenated homophenyl alanine, methylphenyl alanine, nitrophenyl alanine, alkyl tyrosine, phosphotyrosine, mono-, di-, tri-, and tetrahalogenated tyrosyl, substituted and unsubstituted, especially mono-, di-, tri-, and tetrahalogenated and alkylated homotyrosyl, substituted and unsubstituted, especially halogenated 4-biphenyl alanine, diphenyl glycine, 2-indanyl glycine, diphenyl alanine, 4-benzoyl phenyl alanine, 3-benzothienyl alanine.

An amino group within the meaning of the present invention is a group selected from NH₂, NHR′ and NR′R″ wherein the R′ and R″ groups are selected independently from alkyl, alkenyl, and aryl, preferably C₁-C₄ alkyl, C₂-C₆ alkenyl and C₆-C₁₄ aryl. NH₂, dimethyl amine and diethyl amine are especially preferred.

Acid groups in the side chain within the meaning of the present invention are groups of which at least 5% are present in a deprotonated state in an aqueous solution at a pH value of 7.

Basic groups in the side chain within the meaning of the present invention are groups of which at least 5% are present in a protonated state in an aqueous solution at a pH value of 7. A side chain is a basic side chain if at least one basic group is contained. Polyfunctional side chains are defined as basic side chains within the meaning of the present invention if they bear more basic groups than acidic groups.

2. The Somatostatin Derivatives of the Present Invention.

The peptides of the present invention are represented by the general formulae 1 to 6.

y₁-Aₙ-B—C-Dₘ-Z-y² (1)

y¹-Z-Aₙ-B—C-Dₘ-y² (2)

y¹-Dₘ-Z-Aₙ-B—C-y² (3)

y¹-C-Dₘ-Z-Aₙ-B-y² (4)

y¹-B—C-Dₘ-Z-Aₙ-y² (5)

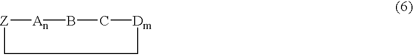

The groups A, B, C, D, and Z are radicals derived from aminocarboxylic acids linked to each other by a peptide linkage. n and m represent 0 or 1 and n+m represents 1 or 2. Accordingly, the formulae 1 to 6 represent tetra- or pentapeptides.

The linear peptides of the formulae 1 to 5 may be derived from the cyclic peptide of the formula 6 by cleaving any binding site among the peptide linkages and by saturating the free valences with the terminal groups y¹ and y².

Group Z is described by the following general formula 7

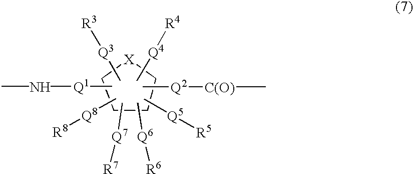

wherein the substituents Q¹, Q², Q³, Q⁴, Q⁵, Q⁶, Q⁷, Q¹, R³, R⁴, R⁵, R⁶, R⁷, R⁸ and X have the following meaning:

X is selected from O, S, Se, NR⁹, PR⁸ and CR⁹R¹⁰, preferably O and NH, wherein R⁹, R¹⁰ are independently selected from H, OH, SH, F, Cl, Br, I, alkyl, alkenyl, alkinyl, aryl, alkylaryl, arylalkyl, alkoxy, alkenyloxy, aryloxy, thioalkyl, thioaryl, selenoalkyl, selenoaryl which may optionally be substituted with one or more of the substituents selected from F, OH, SH, SeH, an amino group, an oxo group and a carboxy group. H, alkyl, aryl and OH are preferred.

Q¹ and Q² are independently selected from a single bond, CH₂, CH(OH), CH(OR¹), CHR¹ and CR¹R²,

wherein R¹ and R² are independently selected from alkyl, alkenyl, aryl, arylalkyl, alkylaryl, which may optionally be substituted with F, OH, an amino group or a carboxy group.

Preferred groups Q¹ and Q² are a single bond, CH(OH) and CH(O benzyl), especially mono-, di-, tri-, tetra- and pentahalogenated benzyl ether, fluorinated benzyl ether, alkylated benzyl ether, arylbenzyl ether, hydroxy benzyl ether and alkoxy benzyl ether.

Q³ bis Q⁸ are independently selected from a single bond, O, S, Se, N₂, NR⁹, PO₃.

R³ bis R⁸ are independently selected from the group consisting of H, OH, SH, N₃, CN, NC, SCN, F, Cl, Br, I, SO₃, NO₂, PR¹¹R¹², COOR¹¹, alkyl, alkenyl, alkinyl, aryl, alkylaryl, arylalkyl, alkanoyl, alkenoyl, alkinoyl, aroyl, arylalkanoyl, alkylaroyl, which may optionally be substituted with one or more substituents selected from F, OH, SH, SeH, an amino group, an oxo group or a carboxy group.

R¹¹ and R¹² are independently selected from H, OH, SH, F, Cl, Br, I, CN, NC, SCN, alkyl, alkenyl, alkinyl, aryl, alkylaryl, arylalkyl, alkoxy, alkenyloxy, aryloxy, thioalkyl, thioalkenyl, thioaryl, selenoalkyl, selenoalkenyl, selenoaryl, amidoalkyl, amidoalkenyl, amidoalkinyl, arylalkanoyloxy, alkylaroyloxy, arylalkoxy, alkylaryloxy, which may optionally be substituted with one or more of the substituents selected from F, OH, SH, SeH, an amino group, an oxo group or a carboxy group.

Optionally, two substituents Rⁱ and R^{j}, with i, j=3 to 8, are linked, forming a 5- or 6-membered ring, wherein optionally one or more of the ring atoms are independently substituted with one or more groups, independently selected from alkyl, alkenyl and aryl. Typical representatives of this group are spiro compounds, aryl ketals, alkylaryl ketals, alkyl acetals, aryl acetals, arylthio ketals, alkylarylthio ketals, alkylthio acetals, arylthio acetals, aryl aminals, alkylaryl aminals, alkyl aminals and aryl aminals each of which may be substituted or unsubstituted, branched or unbranched. Alkyl ketals, aryl ketals, alkylaryl ketals, alkyl acetals or aryl acetals are preferred. The ketal of acetone and the ketal of substituted or unsubstituted benzophenone are especially preferred.

Preferred substituents -Qⁱ-Rⁱ and -Q^{j}-R^{j}, with i, j=3 to 8, are H, alkyl, alkenyl, aryl, arylalkyl, alkylaryl, alkoxy, aryloxy, aroyloxy and alkanoyloxy. Especially preferred are H, methoxy, benzyloxy, allyloxy and —O—C(CH₃)₂—O—.

It is also especially preferred to select the substituents -Qⁱ-Rⁱ with i 3 to 8, in such a manner that each of the ring atoms in formula (7) except X bears a hydrogen atom and a substituent other than hydrogen. This criterion is met by most of the monosaccharides occurring in nature. The use of such molecules as starting materials provides the advantage that the groups Z with a defined stereochemistry may be obtained at low cost.

Preferred regioisomers of group Z are characterised in that the groups -Q¹-NH— and -Q²-C(O)— are linked to adjacent carbon atoms of the ring in formula (7). Z groups wherein the groups -Q¹-NH— and -Q²-C(O)— are linked to the two carbon atoms of the ring in formula (7) which are adjacent to X are also preferrred.

The structural formulae for preferred representatives of group Z are shown in the following. In each case, the free amino carboxylic acids are shown. In the peptide of the invention, peptide linkages are present at the positions of the amino group and of the carboxyl group. The substituents R, R′ and R″ shown in the following structural images have the same meaning as the substituents -Qⁱ-Rⁱ, wherein i=3 to 8, defined above and in the claims.

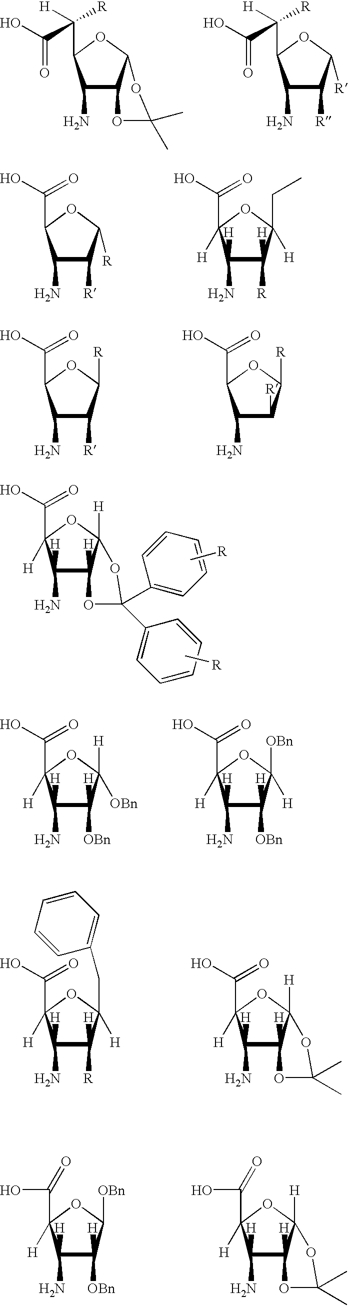

as well as

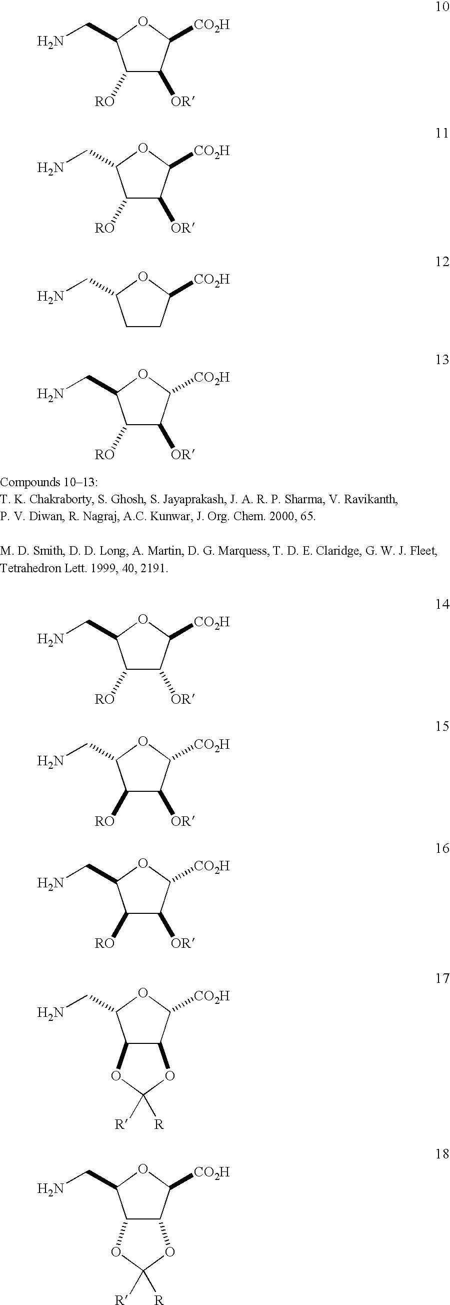

Group A is an α-, β- or γ-amino carboxylic acid radical having an aromatic side chain or an aliphatic side chain. C₆-C₁₄ aryl groups, which may optionally be substituted with OH or I and wherein a carbon atom may be isosterically replaced by nitrogen or sulfur, and C₁-C₁₀ alkyl groups are preferred. It is also preferred if the side chain of the amino carboxylic radical A is a C₁-C₄ alkyl-C₆-C₁₄ aryl group wherein the aryl group is optionally substituted with OH or I and wherein a carbon atom may optionally be replaced isosterically by nitrogen or sulfur.

The amino carboxylic acid radicals of valine, tyrosine, the methyl ether of tyrosine and of phenyl alanine are particularly preferred. Also preferred is D-Asp incorporated as a β-amino acid wherein the side chain is amidically linked to benzyl amine or 1-naphthyl amine via an amide linkage. Also preferred are β-Phe, β-Tyr and β-Val wherein the side-chain may be located in the 2- or 3-position. With regard to the nomenclature and synthesis of β-amino carboxylic acids reference is made to the works of D. Seebach: Helv. Chim. Acta 1998, 81, 2141; Angewandte Chemie 1999, 111, 1302; Helv. Chim. Acta 2000, 83, 16; Helv. Chim. Acta 1998, 81, 187; Helv. Chim. Acta 1998, 81, 983; Helv. Chim. Acta 1998, 81, 2093; Helv. Chim. Acta 1999, 82, 1150; Liebigs Ann. Chem. 1995, 1217; Helv. Chim. Acta 2000, 83, 3139; Helv. Chim. Acta 1996, 79, 913; Helv. Chim. Acta 1996, 79, 2043; Helv. Chim. Acta 1997, 80, 2033; Helv. Chim. Acta 1998; 81; 2218; Chimia 1998, 52, 734.

B is an α-, β- or γ-amino carboxylic acid radical having an aromatic side chain. Side chains having a C₆-C₁₄ aryl group or a C₁-C₄ alkyl-C₆-C₁₄ aryl group which may optionally be substituted with OH or I and wherein a carbon atom may optionally be replaced isosterically by nitrogen or sulfur are preferred.

Especially preferred are the amino carboxylic acid radicals of 1-naphthyl alanine, 2-naphthyl alanine, Bta and tryptophan. In each of these cases, the D- and L-forms of the radicals are preferred.

C is an α-, β- or γ-amino carboxylic acid radical having a basic side chain or an aliphatic side chain. Preferably, the side chain is a C₁-C₁₀ alkyl group which may be substituted with one or more groups selected from amino, acetyl, trifluoroacetyl and alkyl amide groups. Especially preferred are side chains having a C₃-C₅ alkyl group or a C₃-C₅ amino alkyl group.

Especially preferred representatives of group C are the radicals of the amino carboxylic acids lysine, acetal protected lysine and norleucine.

D is an α-, β- or γ-amino carboxylic acid radical which does not have acidic groups or basic groups in the side chain. Side chains having a C₆-C₁₄ aryl group or a C₁-C₄ alkyl-C₆-C₁₄ aryl group which may optionally be substituted with OH or I and wherein a carbon atom may optionally be replaced isosterically by nitrogen or sulfur are preferred. Also preferred are radicals wherein the side chain is a C₁-C₆ alkyl group which may optionally be substituted with one or more groups selected from OH, C₁-C₁₀ alkoxy, C₆-C₂₀ aryl-C₁-C₄ alkoxy, and C₆-C₂₀ aryloxy.

Preferred representatives of this group are the radicals of the amino carboxylic acids Bip, Bpa, Dip, 1-Nal, 2-Nal and threonine.

Especially preferred are the radicals of the threonine ethers and tyrosine ethers where the ether is formed from threonine or tyrosine and an aromatic group or an arylalkyl group. Preferred representatives of this group are trityl ether, benzyl ether and the Phe(F₅) ether of threonine and the trityl ether, benzyl ether and the Phe(F₅) ether of tyrosine.

Also preferred are side chains where an aryl group or an aralkyl group is linked to the backbone of the peptide by an amide linkage. Preferred representatives are D- and L-Asp incorporated as a β- or α-amino acid which is peptidically linked to aminopyrene, 1-naphthyl amine, benzyl amine, anthraquinone amine via the second acidic group.

In addition, the linear peptides comprise the end groups y¹ and y².

y¹ is linked to the amino group of the corresponding amino carboxylic acid and is selected from H, CH₃(CH₂)ᵣCO, with r=0 to 6, butoxy carbonyl and 9-fluorenyl methoxy carbonyl. Preferred groups are acetyl and trifluoro acetyl.

y² is linked to the carboxy group of the corresponding amino carboxylic acid and is selected from H, NH₂, alkoxy, aryloxy, alkyl, aryl, alkenyl, alkinyl, F, Cl, Br, I, CN, NC, SCN, thioalkyl, thioaryl. Preferred groups are NH₂, methoxy, ethoxy and benzyloxy.

Each of n and m represent the integers 0 or 1, such that m+n is 1 or 2:

Preferred sequences of the peptide are those listed in the following:

cyclo[-Phe-Trp-Lys-Z-], cyclo[-Phe-D-Trp-Lys-Z-], cyclo[-Phe-Trp-Nle-Z-], cyclo[-Phe-D-Trp-Nle-Z-], cyclo[-Tyr-Trp-Lys-Z-], cyclo[-Tyr-D-Trp-Lys-Z-], cyclo[-Tyr-Trp-Nle-Z-], cyclo[-Tyr-D-Trp-Nle-Z-], cyclo[-Phe-Bta-Lys-Z-], cyclo[-Phe-D-Bta-Lys-Z-], cyclo[-Phe-Bta-Nle-Z-], cyclo[-Phe-D-Bta-Nle-Z-], cyclo[-Tyr-Bta-Lys-Z-], cyclo[-Tyr-D-Bta-Lys-Z-], cyclo[-Tyr-Bta-Nle-Z-], cyclo[-Tyr-D-Bta-Nle-Z-], cyclo[-Phe-1-Nal-Lys-Z-], cyclo[-Phe-D-1-Nal-Lys-Z-], cyclo[-Phe-1-Nal-Nle-Z-], cyclo[-Phe-D-1-Nal-Nle-Z-], cyclo[-Tyr-1-Nal-Lys-Z-], cyclo[-Tyr-D-1-Nal-Lys-Z-], cyclo[-Tyr-1-Nal-Nle-Z-], cyclo[-Tyr-D-1-Nal-Nle-Z-], cyclo[-Phe-2-Nal-Lys-Z-], cyclo[-Phe-D-2-Nal-Lys-Z-], cyclo[-Phe-2-Nal-Nle-Z-], cyclo[-Phe-D-2-Nal-Nle-Z-], cyclo[-Tyr-2-Nal-Lys-Z-], cyclo[-Tyr-D-2-Nal-Lys-Z-], cyclo[-Tyr-2-Nal-Nle-Z-], cyclo[-Tyr-D-2-Nal-Nle-Z-], cyclo[-Tyr(Bzl)-Bta-Lys-Z-], cyclo[-Tyr(Bzl)-D-Bta-Lys-Z-], cyclo[-Tyr(Bzl)-Bta-Nle-Z-], cyclo[-Tyr(Bzl)-D-Bta-Nle-Z-], cyclo[-Tyr(Bzl)-1-Nal-Lys-Z-], cyclo[-Tyr(Bzl)-D-1-Nal-Lys-Z-], cyclo[-Tyr(Bzl)-1-Nal-Nle-Z-], cyclo[-Tyr(Bzl)-D-1-Nal-Nle-Z-], cyclo[-Tyr(Bzl)-2-Nal-Lys-Z-], cyclo[-Tyr(Bzl)-D-2-Nal-Lys-Z-], cyclo[-Tyr(Bzl)-2-Nal-Nle-Z-], cyclo[-Tyr(Bzl)-D-2-Nal-Nle-Z-], cyclo[-Phe-Trp-Lys-Phe-Z-], cyclo[-Phe-D-Trp-Lys-Phe-Z-], cyclo[-Tyr-Trp-Lys-Phe-Z-], cyclo[-Tyr-D-Trp-Lys-Phe-Z-], cyclo[-Tyr(Me)-Trp-Lys-Phe-Z-], cyclo[-Tyr(Me)-D-Trp-Lys-Phe-Z-], cyclo[-Phe-Trp-Lys-Thr-Z-], cyclo[-Phe-D-Trp-Lys-Thr-Z-], cyclo[-Phe-Trp-Lys-Tyr(Bzl)-Z-], cyclo[-Phe-D-Trp-Lys-Tyr(Bzl)-Z-], cyclo[-Phe-Trp-Lys-Bip-Z-], cyclo[-Phe-D-Trp-Lys-Bip-Z-], cyclo[-Phe-Trp-Lys-Dip-Z-], cyclo[-Phe-D-Trp-Lys-Dip-Z-], cyclo[-Phe-Trp-Lys-Bpa-Z-], cyclo[-Phe-D-Trp-Lys-Bpa-Z-], cyclo[-Phe-Trp-Lys-1-Nal-Z-], cyclo[-Phe-D-Trp-Lys-1-Nal-Z-], cyclo[-Phe-T r-Lys-2-Nal-Z-], cyclo[-Phe-D-Trp-Lys-2-Nal-Z-], cyclo[-Phe-Trp-Lys-p-fluoro-Phe-Z-], cyclo[-Phe-D-Trp-Lys-p-fluoro-Phe-Z-], cyclo[-Phe-Trp-Lys-Phe(F5)-Z-], cyclo[-Phe-D-Trp-Lys-Phe(F5)-Z-], cyclo[-Phe-Trp-Lys-o-fluoro-Phe-Z-], cyclo[-Phe-D-Trp-Lys-o-fluoro-Phe-Z-], cyclo[-Phe-Trp-Lys-m-fluoro-Phe-Z-], cyclo[-Phe-D-Trp-Lys-m-fluoro-Phe-Z-], cyclo[-Phe-Trp-Lys-Thr(Ar)-Z-], cyclo[-Phe-D-Trp-Lys-Thr(Ar)-Z-], cyclo[-Phe-Trp-Lys-Thr(Bn)-Z-], cyclo[-Phe-D-Trp-Lys-Thr(Bn)-Z-], cyclo[-Phe-Trp-Lys-2,4-difluoro-Phe-Z-], cyclo[-Phe-D-Trp-Lys-2,4-difluoro-Phe-Z-], cyclo[-Phe-Trp-Lys-2,3-difluoro-Phe-Z-], cyclo[-Phe-D-Trp-Lys-2,3-difluoro-Phe-Z-], cyclo[-Phe-Trp-Lys-2,5-difluoro-Phe-Z-], cyclo[-Phe-D-Trp-Lys-2,5-difluoro-Phe-Z-], cyclo[-Phe-Trp-Lys-p-chloro-Phe-Z-], cyclo[-Phe-D-Trp-Lys-p-chloro-Phe-Z-], cyclo[-Phe-Trp-Lys-Phe(C15)-Z-], cyclo[-Phe-D-Trp-Lys-Phe(C15)-Z-], cyclo[-Phe-Trp-Lys-o-chloro-Phe-Z-], cyclo[-Phe-D-Trp-Lys-o-chloro-Phe-Z-], cyclo[-Phe-Trp-Lys-m-chloro-Phe-Z-], cyclo[-Phe-D-Trp-Lys-m-chloro-Phe-Z-], cyclo[-Phe-Trp-Lys-Thr(Ar)-Z-], cyclo[-Phe-Trp-Lys-2,4-dichloro-Phe-Z-], cyclo[-Phe-D-Trp-Lys-2,4-dichloro-Phe-Z-], cyclo[-Phe-Trp-Lys-2,3-dichloro-Phe-Z-], cyclo[-Phe-D-Trp-Lys-2,3-dichloro-Phe-Z-], cyclo[-Phe-Trp-Lys-2,5-dichloro-Phe-Z-], cyclo[-Phe-D-Trp-Lys-2,5-dichloro-Phe-Z-], cyclo[-Phe-Trp-Lys-3,5-dichloro-Phe-Z-], cyclo[-Phe-D-Trp-Lys-3,4-dichloro-Phe-Z-], cyclo[-Phe-Trp-Lys-3,4-dichloro-Phe-Z-], cyclo[-Phe-D-Trp-Lys-3,5-dichloro-Phe-Z-], cyclo[Phe-Trp-Nle-Phe-Z], cyclo[-Phe-D-Trp-Nle-Phe-Z-], cyclo[-Tyr-Trp-Nle-Phe-Z-], cyclo[-Tyr-D-Trp-Nle-Phe-Z-], cyclo[-Tyr(Me)-Trp-Nle-Phe-Z-], cyclo[-Tyr(Me)-D-Trp-Nle-Phe-Z-], cyclo[-Phe-Trp-Nle-Thr-Z-], cyclo[-Phe-D-Trp-Nle-Thr-Z-], cyclo[-Phe-Trp-Nle-Bip-Z-], cyclo[-Phe-D-Trp-Nle-Bip-Z-], cyclo[-Phe-Trp-Nle-Dip-Z-], cyclo[-Phe-D-Trp-Nle-Dip-Z-], cyclo[-Phe-Trp-Nle-Bpa-Z-], cyclo[-Phe-D-Trp-Nle-Bpa-Z-], cyclo[-Phe-Trp-Nle-1-Nal-Z-], cyclo[-Phe-D-Trp-Nle-1-Nal-Z-], cyclo[-Phe-Trp-Nle-2-Nal-Z-], cyclo[-Phe-D-Trp-Nle-2-Nal-Z-], cyclo[-Phe-Trp-Nle-p-fluoro-Phe-Z-], cyclo[-Phe-D-Trp-Nle-p-fluoro-Phe-Z-], cyclo[-Phe-Trp-Nle-Phe(F5)-Z-], cyclo[-Phe-D-Trp-Nle-Phe(F5)-Z-], cyclo[-Phe-Trp-Nle-o-fluoro-Phe-Z-], cyclo[-Phe-D-Trp-Nle-o-fluoro-Phe-Z-], cyclo[-Phe-Trp-Nle-m-fluoro-Phe-Z-], cyclo[-Phe-D-Trp-Nle-m-fluoro-Phe-Z-], cyclo[-Phe-Trp-Nle-Thr(Ar)-Z-], cyclo[-Phe-D-Trp-Nle-Thr(Ar)-Z-], cyclo[-Phe-Trp-Nle-Thr(Bn)-Z-], cyclo[-Phe-D-Trp Nle-Thr(Bn)-Z-], cyclo[-Phe-Trp-Nle-2,4-difluoro-Phe-Z-], cyclo[-Phe-D-Trp-Nle-2,4-difluoro-Phe-Z], cyclo[-Phe-Trp-Nle-2,3-difluoro-Phe-Z-], cyclo[-Phe-D-Trp-Nle-2,3-difluoro-Phe-Z-], cyclo[-Phe-Trp-Nle 2,5-difluoro-Phe-Z-], cyclo[-Phe-D-Trp-Nle-2,5-difluoro-Phe-Z-], cyclo[-Phe-Trp-Nle-p-chloro-Phe-Z-], cyclo[-Phe-D-Trp-Nle-p-chloro-Phe-Z-], cyclo[-Phe-Trp-Nle-Phe(C15)-Z-], cyclo[-Phe-D-Trp-Nle-Phe(C15)-Z-], cyclo[-Phe-Trp-Nle-o-chloro-Phe-Z-], cyclo[-Phe-D-Trp-Nle-o-chloro-Phe-Z-], cyclo[-Phe-Trp-Nle-m-chloro-Phe-Z-], cyclo[-Phe-D-Trp-Nle-m-chloro-Phe-Z-], cyclo[-Phe-Trp-Nle-Thr(Ar)-Z-], cyclo[-Phe-Trp-Nle-2,4-dichloro-Phe-Z-], cyclo[-Phe-D-Trp-Nle-2,4-dichloro-Phe-Z-], cyclo[-Phe-Trp-Nle-2,3-dichloro-Phe-Z-], cyclo[-Phe-D-Trp-Nle-2,3-dichloro-Phe-Z-], cyclo[-Phe-Trp-Nle-2,5-dichloro-Phe-Z-], cyclo[-Phe-D-Trp-Nle-2,5-dichloro-Phe-Z-], cyclo[-Phe-Trp-Nle-3,5-dichloro-Phe-Z-], cyclo[-Phe-D-Trp-Nle-3,4-dichloro-Phe-Z-], cyclo[-Phe-Trp-Nle-3,4-dichloro-Phe-Z-], cyclo[-Phe-D-Trp Nle-3,5-dichloro-Phe-Z-], cyclo[-Phe-Trp-Nle-Nle(6-OBzl)-Z-], cyclo[-Phe-D-Trp Nle-Nle(6-OBzl)-Z-], cyclo[-Tyr-Trp-Nle-Nle(6-OBzl)-Z-], cyclo[-Tyr-D-Trp-Nle-Nle(6-OBzl)-Z-], cyclo[-Tyr(Me)-Trp-Nle-Nle(6-OBzl)-Z-], cyclo[-Phe-Trp-Nle-3-Pal-Z-], cyclo[-Phe-D-Trp-Nle-3-Pal-Z-], cyclo[-Tyr-Trp-Nle-3-Pal-Z-], cyclo[-Tyr-D-Trp-Nle-3-Pal-Z-], cyclo[-Tyr(Me)-Trp-Nle-3-Pal-Z-], cyclo[-Tyr(Me)-D-Trp-Nle-3-Pal-Z-], cyclo[-Phe-Trp-Nle-4-Pal-Z-], cyclo[-Phe-D-Trp-Nle-4-Pal-Z-], cyclo[-Tyr-Trp-Nle-4-Pal-Z-], cyclo[-Tyr-D-Trp-Nle-4-Pal-Z-], cyclo[-Tyr(Me)-Trp-Nle-4-Pal-Z-], cyclo[-Phe-Trp-Nle-3,4-dichloro-Phe-Z-], cyclo[-Phe-D-Trp-Nle-3,4-dichloro-Phe-Z-], cyclo[-Phe-Trp-Nle-3,4-difluoro-Phe-Z-], cyclo[-Phe-D-Trp-Nle-3,4-difluoro-Phe-Z-], cyclo[-Phe-Trp-Nle-Phg-Z-], cyclo[-Phe-D-Trp-Nle-Phg-Z-], cyclo[-Tyr-Trp-Nle-Phg-Z-], cyclo[-Tyr-D-Trp-Nle-Phg-Z-], cyclo[-Tyr(Me)-Trp-Nle-Phg-Z-], cyclo[-Phe-Trp-Nle-Phe-Z-], cyclo[-Phe-D-Trp-Nle-hPhe-Z-], cyclo[-Tyr-Trp-Nle-hPhe-Z-], cyclo[-Tyr-D-Trp-Nle-hPhe-Z-], cyclo[-Tyr(Me)-Trp-Nle-hPhe-Z-], cyclo[-Phe-Trp-Nle-Igl-Z-], cyclo[-Phe-D-Trp-Nle-Igl-Z-], cyclo[-Tyr-Trp-Nle-Igl-Z-], cyclo[-Tyr-D-Trp-Nle-Igl-Z-], cyclo[-Tyr(Me)-Trp-Nle-Igl-Z-], cyclo[-Phe-Trp-Nle-Phe(4-NO2)-Z-], cyclo[-Phe-D-Trp-Nle-Phe(4-NO2)>Z-], cyclo[-Tyr-Trp-Nle-Phe(4-NO2)-Z-], cyclo[-Tyr-D-Trp-Nle-Phe(4-NO2)-Z-], cyclo[-Tyr(Me)-Trp-Nle-Phe(4-NO2)-Z-], cyclo[-Phe-Trp-Nle-Phe(4-NHz)-Z-], cyclo[-Phe-D-Trp-Nle-Phe(4-NHz)-Z-], cyclo[-Tyr-Trp-Nle-Phe(4-NHz)-Z-], cyclo[-Tyr-D-Trp-Nle-Phe(4-NHz)-Z-], cyclo[-Tyr(Me)-Trp-Nle-Phe(4-NHz)-Z-], cyclo[-Phe-Trp-Nle-Phe(4-NH-2Clz)-Z-], cyclo[-Phe-D-Trp-Nle-Phe(4-NH-2Clz)-Z-], cyclo[-Tyr-Trp-Nle-Phe(4-NH-2Clz)-Z-], cyclo[-Tyr-D-Trp-Nle-Phe(4-NH-2Clz)-Z-], cyclo[-Tyr(Me)-Trp-Nle-Phe(4-NH-2Clz)-Z-], cyclo[-Phe-Trp-Nle-hTyr-Z-], cyclo[-Phe-D-Trp-Nle-hTyr-Z-], cyclo[-Tyr-Trp-Nle-hTyr-Z-], cyclo[-Tyr-D-Trp-Nle-hTyr-Z-], cyclo[-Tyr(Me)-Trp-Nle-hTyr-Z-], cyclo[-Phe-Trp-Nle-Pra-Z-], cyclo[-Phe-D-Trp-Nle-Pra-Z-], cyclo[-Tyr-Trp-Nle-Pra-Z-], cyclo[-Tyr-D-Trp-Nle-Pra-Z-], cyclo[-Tyr(Me)-Trp-Nle-Pra-Z-], cyclo[-Phe-1-Nal-Nle-Phe-Z-], cyclo[-Phe-D-1-Nal-Nle-Phe-Z-], cyclo[-Tyr-1-Nal-Nle-Phe-Z-], cyclo[-Tyr-D-1-Nal-Nle-Phe-Z-], cyclo[-Tyr(Me)-1-Nal-Nle-Phe-Z-], cyclo[-Tyr(Me)-D-1-Nal-Nle-Phe-Z-], cyclo[-Phe-1-Nal-Nle-Thr-Z-], cyclo[-Phe-D-1-Nal-Nle-Thr-Z-], cyclo[-Phe-1-Nal-Nle-Tyr(Bzl)-Z-], cyclo[-Phe-D-1-Nal-Nle-Tyr(Bzl)-Z-], cyclo[-Phe-1-Nal-Nle-Bip-Z-], cyclo[-Phe-D-1-Nal-Nle-Bip-Z-], cyclo[-Phe-1-Nal-Nile-Dip-Z-], cyclo[Phe-D-1 Nal-Nle-Dip-Z-], cyclo[-Phe-1-Nal-Nle-Bpa-Z-], cyclo[-Phe-D-1-Nal-Nle-Bpa-Z-], cyclo[-Phe-1-Nal-Nle-1-Nal-Z-], cyclo[-Phe-D-1-Nal-Nle-1-Nal-Z-], cyclo[-Phe-1-Nal-Nle-2-Nal-Z-], cyclo[-Phe-D-1-Nal-Nle-2-Nal-Z-], cyclo[-Phe-1-Nal-Nle-p-fluoro-Phe-Z-], cyclo[-Phe-D-1-Nal-Nle-p-fluoro-Phe-Z-], cyclo[-Phe-1-Nal-Nle-Phe(F5)-Z-], cyclo[-Phe-D-1-Nal-Nle-Phe(F5)-Z-], cyclo[-Phe-1-Nal-Nle-o-fluoro-Phe-Z-], cyclo[-Phe-D-1-Nal-Nle-o-fluoro-Phe-Z-], cyclo[-Phe-1-Nal-Nle-m-fluoro-Phe-Z-], cyclo[-Phe-D-1-Nal-Nle-m-fluoro-Phe-Z-], cyclo[-Phe-1-Nal-Nle-Thr(Ar)-Z-], cyclo[-Phe-D-1-Nal-Nle-Thr(Ar)-Z-], cyclo[-Phe-1-Nal-Nle-Thr(Bn)-Z-], cyclo[-Phe-D-1-Nal-Nle-Thr(Bn)-Z-], cyclo[-Phe-1-Nal-Nle-2,4-difluoro-Phe-Z-], cyclo[-Phe-D-1-Nal-Nle-2,4-difluoro-Phe-Z-], cyclo[-Phe-1-Nal-Nle-2,3-difluoro-Phe-Z-], cyclo[-Phe-D-1-Nal-Nle-2,3-difluoro-Phe-Z-], cyclo[-Phe-1-Nal-Nle-2,5-difluoro-Phe-Z-], cyclo[-Phe-D-1-Nal-Nle-2,5-difluoro-Phe-Z-], cyclo[-Phe-1-Nal-Nle-p-chloro-Phe-Z-], cyclo[-Phe-D-1-Nal-Nle-p-chloro-Phe-Z-], cyclo[-Phe-1-Nal-Nle-Phe(C15)-Z-], cyclo[-Phe-D-1-Nal-Nle-Phe(C15)-Z-], cyclo[-Phe-1-Nal-Nle-o-chloro-Phe-Z-], cyclo[-Phe-D-1-Nal-Nle-o-chloro-Phe-Z-], cyclo[-Phe-1-Nal-Nle-m-chloro-Phe-Z-], cyclo[-Phe-D-1-Nal-Nle-m-chloro-Phe-Z-], cyclo[-Phe-1-Nal-Nle-Thr(Ar)-Z-], cyclo[-Phe-1-Nal-Nle-2,4-dichloro-Phe-Z-], cyclo[-Phe-D-1-Nal-Nle-2,4-dichloro-Phe-Z-], cyclo[-Phe-1-Nal-Nle-2,3-dichloro-Phe-Z-], cyclo[-Phe-D-1-Nal-Nle-2,3-dichloro-Phe-Z-], cyclo[-Phe-1-Nal-Nle-2,5-dichloro-Phe-Z-], cyclo[-Phe-D-1-Nal-Nle-2,5-dichloro-Phe-Z-], cyclo[-Phe-1-Nal-Nle-2,5-dichloro-Phe-Z-], cyclo[-Phe-D-1-Nal-Nle-2,5-dichloro-Phe-Z-], cyclo[-Phe-1-Nal-Nle-3,5-dichloro-Phe-Z-], cyclo[-Phe-D-1-Nal-Nle-3,5-dichloro-Phe-Z-], cyclo[-Phe-1-Nal-Nle-3,5-difluoro-Phe-Z-], cyclo[-Phe-D-1-Nal-Nle-3,5-difluoro-Phe-Z-], cyclo[-Phe-1-Nal-Nle-Nle(6-OBzl)-Z-], cyclo[-Phe-D-1-Nal-Nle-Nle(6-OBzl)-Z-], cyclo[-Tyr-1-Nal-Nle-Nle(6-OBzl)-Z-], cyclo[-Tyr-D-1-Nal-Nle-Nle(6-OBzl)-Z-], cyclo[-Tyr(Me)-1-Nal-Nle-Nle(6-OBzl)-Z-], cyclo[-Tyr(Me)-D-1-Nal-Nle-Nle(6-OBzl)-Z-], cyclo[-Phe-1-Nal-Nle-3,4-dichloro-Phe-Z-], cyclo[-Phe-D-1-Nal-Nle-3,4-dichloro-Phe-Z-], cyclo[-Phe-1-Nal-Nle-3,4-difluoro-Phe-Z-], cyclo[-Phe-D-1-Nal-Nle-3,4-difluoro-Phe-Z-], cyclo[-Phe-1-Nal-Nle-3-Pal-Z-], cyclo[-Phe-D-1-Nal-Nle-3-Pal-Z-], cyclo[-Tyr-1-Nal-Nle-3-Pal-Z-], cyclo[-Tyr-D-1-Nal-Nle-3-Pal-Z-], cyclo[-Tyr(Me)-1-Nal-Nle-3-Pal-Z-], cyclo[-Tyr(Me)-D-1-Nal-Nle-3-Pal-Z-], cyclo[-Phe-1-Nal-Nle-4-Pal-Z-], cyclo[-Phe-D-1-Nal-Nle-4-Pal-Z-], cyclo[-Tyr-1-Nal-Nle-4-Pal-Z-], cyclo[-Tyr-D-1-Nal-Nle-4-Pal-Z-], cyclo[-Tyr(Me)-1-Nal-Nle-4-Pal-Z-], cyclo[-Phe-1-Nal-Nle-3,4-dichloro-Phe-Z-], cyclo[-Phe-D-1-Nal-Nle-3,4-dichloro-Phe-Z-], cyclo[-Phe-1-Nal-Nle-3,4-difluoro-Phe-Z-], cyclo[-Phe-D-1-Nal-Nle-3,4-difluoro-Phe-Z-], cyclo[-Phe-1-Nal-Nle-Phg-Z-], cyclo[-Phe-D-1-Nal-Nle-Phg-Z-], cyclo[Tyr-1-Nal-Nle-Phg-Z-], cyclo[-Tyr-D-1-Nal-Nle-Phg-Z-], cyclo[-Tyr(Me)-1-Nal-Nle-Phg-Z-], cyclo[-Phe-1-Nal-Nle-hPhe-Z-], cyclo[-Phe-D-1-Nal-Nle-hPhe-Z-], cyclo[-Tyr-1-Nal-Nle-hPhe-Z-], cyclo[-Tyr-D-1-Nal-Nle-hPhe-Z-], cyclo[-Tyr(Me)-1-Nal-Nle-hPhe-Z-], cyclo[-Phe-1-Nal-Nle-Igl-Z-], cyclo[-Phe-D-1-Nal-Nle-Igl-Z-], cyclo[-Tyr-1-Nal-Nle-Igl-Z-], cyclo[-Tyr-D-1-Nal-Nle-Igl-Z-], cyclo[-Tyr(Me)-1-Nal-Nle-Igl-Z-], cyclo[-Phe-1-Nal-Nle-Phe(4-NO2)-Z-], cyclo[-Phe-D-1-Nal-Nle-Phe(4-NO2)-Z-], cyclo[-Tyr-1-Nal-Nle-Phe(4-NO2)-Z-], cyclo[-Tyr-D-1-Nal-Nle-Phe(4-NO2)-Z-], cyclo[-Tyr(Me)-1-Nal-Nle-Phe(4-NO2)-Z-], cyclo[-Phe-1-Nal-Nle-Phe(4-NHz)-Z-], cyclo[-Phe-D-1-Nal-Nle-Phe(4-NHz)-Z-], cyclo[-Tyr-1-Nal-Nle-Phe(4-NHz)-Z-], cyclo[-Tyr-D-1-Nal-Nle-Phe(4-NHz)-Z-], cyclo[-Tyr(Me)-1-Nal-Nle-Phe(4-NHz)-Z-], cyclo[-Phe-1-Nal-Nle-Phe(4-NH-2Clz)-Z-], cyclo[-Phe-D-1-Nal-Nle-Phe(4-NH-2Clz)-Z-], cyclo[-Tyr-1-Nal-Nle-Phe(4-NH-2Clz)-Z-], cyclo[-Tyr-D-1-Nal-Nle-Phe(4-NH-2Clz)-Z-], cyclo[-Tyr(Me)-1-Nal-Nle-Phe(4-NH-2Clz)-Z-], cyclo[-Phe-1-Nal-Nle-hTyr-Z-], cyclo[-Phe-D-1-Nal-Nle-hTyr-Z-], cyclo[-Tyr-1-Nal-Nle-hTyr-Z-], cyclo[-Tyr-D-1-Nal-Nle-hTyr-Z-], cyclo[-Tyr(Me)-1-Nal-Nle-hTyr-Z-], cyclo[-Phe-1-Nal-Nle-Pra-Z-], cyclo[-Phe-D-1-Nal-Nle-Pra-Z-], cyclo[-Tyr-1-Nal-Nle-Pra-Z-], cyclo[-Tyr-D-1-Nal-Nle-Pra-Z-], cyclo[-Tyr(Me)-1-Nal-Nle-Pra-Z-] cyclo[-Phe-2-Nal-Nle-Phe-Z-], cyclo[-Phe-D-2-Nal-Nle-Phe-Z-], cyclo[-Tyr-2-Nal-Nle-Phe-Z-], cyclo[-Tyr-D-2-Nal-Nle-Phe-Z-], cyclo[-Tyr(Me)-2-Nal-Nle-Phe-Z-], cyclo[-Tyr(Me)-D-2-Nal-Nle-Phe-Z-], cyclo[-Phe-2-Nal-Nle-Thr-Z-], cyclo[-Phe-D-2-Nal-Nle-Thr-Z-], cyclo[-Phe-2-Nal-Nle-Tyr(Bzl)-Z-], cyclo[-Phe-D-2-Nal-Nle-Tyr(Bzl)-Z-], cyclo[-Phe-2-Nal-Nle-Bip-Z-], cyclo[-Phe-D-2-Nal-Nle-Bip-Z-], cyclo[-Phe-2-Nal-Nle-Dip-Z-], cyclo[-Phe-D-2-Nal-Nle-Dip-Z-], cyclo[-Phe-2-Nal-Nle-Bpa-Z-], cyclo[-Phe-D-2-Nal-Nle-Bpa-Z-], cyclo[-Phe-2-Nal-Nle-2-Nal-Z-], cyclo[-Phe-D-2-Nal-Nle-2-Nal-Z-], cyclo[-Phe-2-Nal-Nle-1-Nal-Z-], cyclo[-Phe-D-2-Nal-Nle-1-Nal-Z-], cyclo[-Phe-2-Nal-Nle-p-fluoro-Phe-Z-], cyclo[-Phe-D-2-Nal-Nle-p-fluoro-Phe-Z-], cyclo[-Phe-2-Nal-Nle-Phe(F5)-Z-], cyclo[-Phe-D-2-Nal-Nle-Phe(F5)-Z-], cyclo[-Phe-2-Nal-Nle-o-fluoro-Phe-Z-], cyclo[-Phe-D-2-Nal-Nle-o-fluoro-Phe-Z-], cyclo[-Phe-2-Nal-Nle-m-fluoro-Phe-Z-], cyclo[-Phe-D-2-Nal-Nle-m-fluoro-Phe-Z-], cyclo[-Phe-2-Nal-Nle-Thr(Ar)-Z-], cyclo[-Phe-D-2-Nal-Nle-Thr(Ar)-Z-], cyclo[-Phe-2-Nal-Nle-Thr(Bn)-Z-], cyclo[-Phe-D-2-Nal-Nle-Thr(Bn)-Z-], cyclo[-Phe-2-Nal-Nle-2,4-difluoro-Phe-Z-], cyclo[-Phe-D-2-Nal-Nle-2,4-difluoro-Phe-Z-], cyclo[-Phe-2-Nal-Nle-2,3-difluoro-Phe-Z-], cyclo[-Phe-D-2-Nal-Nle-2,3-difluoro-Phe-Z-], cyclo[-Phe-2-Nal-Nle-2,5-difluoro-Phe-Z-], cyclo[-Phe-D-2-Nal-Nle-2,5-difluoro-Phe-Z-], cyclo[-Phe-2-Nal-Nle-p-chloro-Phe-Z-], cyclo[-Phe-D-2-Nal-Nle-p-chloro-Phe-Z-], cyclo[-Phe-2-Nal-Nle-Phe(C15)-Z-], cyclo[-Phe-D-2-Nal-Nle-Phe(C15)-Z-], cyclo[-Phe-2-Nal-Nle-o-chloro-Phe-Z-], cyclo[-Phe-D-2-Nal-Nle-chloro-Phe-Z-], cyclo[-Phe-2-Nal-Nle-m-chloro-Phe-Z-], cyclo[-Phe-D-2-Nal-Nle-m-chloro-Phe-Z-], cyclo[-Phe-2-Nal-Nle-Thr(Ar)-Z-], cyclo[-Phe-2-Nal-Nle-2,4-dichloro-Phe-Z-], cyclo[-Phe-D-2-Nal-Nle-2,4-dichloro-Phe-Z-], cyclo[-Phe-2-Nal-Nle-2,3-dichloro-Phe-Z-], cyclo[-Phe-D-2-Nal-Nle-2,3-dichloro-Phe-Z-], cyclo[-Phe-2-Nal-Nle-2,5-dichloro-Phe-Z-], cyclo[-Phe-D-2-Nal-Nle-2,5-dichloro-Phe-Z-], cyclo[-Phe-2-Nal-Nle-2,5-dichloro-Phe-Z-], cyclo[-Phe-D-2-Nal-Nle-2,5-dichloro-Phe-Z-], cyclo[-Phe-2-Nal-Nle-3,5-dichloro-Phe-Z-], cyclo[-Phe-D-2-Nal-Nle-3,5-dichloro-Phe-Z-], cyclo[-Phe-2-Nal-Nle-3,5-difluoro-Phe-Z-], cyclo[-Phe-D-2-Nal-Nle-3,5-difluoro-Phe-Z-], cyclo[-Phe-2-Nal-Nle-Nle(6-OBzl)-Z-], cyclo[-Phe-D-2-Nal-Nle-Nle(6-OBzl)-Z-], cyclo[-Tyr-2-Nal-Nle-Nle(6-OBzl)-Z-], cyclo[-Tyr-D-2-Nal-Nle-Nle(6-OBzl)-Z-], cyclo[-Tyr(Me)-2-Nal-Nle-Nle(6-OBzl)-Z-], cyclo[-Tyr(Me)-D-2-Nal-Nle-Nle(6-OBzl)-Z-], cyclo[-Phe-2-Nal-Nle-3,4-dichloro-Phe-Z-], cyclo[-Phe-D-2-Nal-Nle-3,4-dichloro-Phe-Z-], cyclo[-Phe-2-Nal-Nle-3,4-difluoro-Phe-Z-], cyclo[-Phe-D-2-Nal-Nle-3,4-difluoro-Phe-Z-], cyclo[-Phe-2-Nal-Nle-3-Pal-Z-], cyclo[-Phe-D-2-Nal-Nle-3-Pal-Z-], cyclo[-Tyr-2-Nal-Nle-3-Pal-Z-], cyclo[Tyr-D-2-Nal-Nle-3-Pal-Z-], cyclo[-Tyr(Me)-2-Nal-Nle-3-Pal-Z-], cyclo[-Tyr(Me)-D-2-Nal-Nle-3-Pal-Z-], cyclo[-Phe-2-Nal-Nle-4-Pal-Z-], cyclo[-Phe-D-2-Nal-Nle-4-Pal-Z-], cyclo[-Tyr-2-Nal-Nle-4-Pal-Z-], cyclo[-Tyr-D-2-Nal-Nle-4-Pal-Z-], cyclo[-Tyr(Me)-2-Nal-Nle-4-Pal-Z-], cyclo[-Phe-2-Nal-Nle-3,4-dichloro-Phe-Z-], cyclo[-Phe-D-2-Nal-Nle-3,4-dichloro-Phe-Z-], cyclo[-Phe-2-Nal-Nle-3,4-difluoro-Phe-Z-], cyclo[-Phe-D-2-Nal-Nle-3,4-difluoro-Phe-Z-], cyclo[-Phe-2-Nal-Nle-Phg-Z-], cyclo[-Phe-D-2-Nal-Nle-Phg-Z-], cyclo[-Tyr-2-Nal-Nle-Phg-Z-], cyclo[Tyr-D-2-Nal-Nle-Phg-Z-], cyclo[-Tyr(Me)-2-Nal-Nle-Phg-Z-], cyclo[-Phe-2-Nal-Nle-hPhe-Z-], cyclo[-Phe-D-2-Nal-Nle-hPhe-Z-], cyclo[-Tyr-2-Nal-Nle-hPhe-Z-], cyclo[-Tyr-D-2-Nal-Nle-hPhe-Z-], cyclo[-Tyr(Me)-2-Nal-Nle-hPhe-Z-], cyclo[-Phe-2-Nal-Nle-Igl-Z-], cyclo[-Phe-D-2-Nal-Nle-Igl-Z-], cyclo[-Tyr-2-Nal-Nle-Igl-Z-], cyclo[-Tyr-D-2-Nal-Nle-Igl-Z-], cyclo[-Tyr(Me)-2-Nal-Nle-Igl-Z-], cyclo[-Phe-2-Nal-Nle-Phe(4-NO2)-Z-], cyclo[-Phe-D-2-Nal-Nle-Phe(4-NO2)-Z-], cyclo[-Tyr-2-Nal-Nle-Phe(4-NO2)-Z-], cyclo[Tyr-D-2-Nal-Nle-Phe(4-NO2)-Z-], cyclo[Tyr(Me)-2-Nal-Nle-Phe(4-NO2)-Z-], cyclo[-Phe-2-Nal-Nle-Phe(4-NHz)-Z-], cyclo[-Phe-D-2-Nal-Nle-Phe(4-NHz)-Z-], cyclo[-Tyr-2-Nal-Nle-Phe(4-NHz)-Z-], cyclo[-Tyr-D-2-Nal-Nle-Phe(4-NHz)-Z-], cyclo[-Tyr(Me)-2-Nal-Nle-Phe(4-NHz)-Z-], cyclo[-Phe-2-Nal-Nle-Phe(4-NH-2Clz)-Z-], cyclo[-Phe-D-2-Nal-Nle-Phe(4-NH-2Clz)-Z-], cyclo[-Tyr-2-Nal-Nle-Phe(4-NH-2Clz)-Z-], cyclo[Tyr-D-2-Nal-Nle-Phe(4-NH-2Clz)-Z-], cyclo[-Tyr(Me)-2-Nal-Nle-Phe(4-NH-2Clz)-Z-], cyclo[-Phe-2-Nal-Nle-hTyr-Z-], cyclo[-Phe-D-2-Nal-Nle-hTyr-Z-], cyclo[-Tyr-2-Nal-Nle-hTyr-Z-], cyclo[-Tyr-D-2-Nal-Nle-hTyr-Z-], cyclo[-Tyr(Me)-2-Nal-Nle-hTyr-Z-], cyclo[-Phe-2-Nal-Nle-Pra-Z-], cyclo[-Phe-D-2-Nal-Nle-Pra-Z-], cyclo[-Tyr-2-Nal-Nle-Pra-Z-], cyclo[-Tyr-D-2-Nal-Nle-Pra-Z-], cyclo[-Tyr(Me)-2-Nal-Nle-Pra-Z-], cyclo[-Phe-Bta-Nle-Phe-Z-], cyclo[-Phe-D-Bta-Nle-Phe-Z-], cyclo[-Tyr-Bta-Nle-Phe-Z-], cyclo[-Tyr-D-Bta-Nle-Phe-Z-], cyclo[-Tyr(Me)-Bta-Nle-Phe-Z-], cyclo[-Tyr(Me)-D-Bta-Nle-Phe-Z-], cyclo[-Phe-Bta-Nle-Thr-Z-], cyclo[-Phe-D-Bta-Nle-Thr-Z-], cyclo[-Phe-Bta-Nle-Tyr(Bzl)-Z-], cyclo[-Phe-D-Bta-Nle-Tyr(Bzl)-Z-], cyclo[-Phe-Bta-Nle-Bip-Z-], cyclo[-Phe-D-Bta-Nle-Bip-Z-], cyclo[-Phe-Bta-Nle-Dip-Z-], cyclo[-Phe-D-Bta-Nle-Dip-Z-], cyclo[-Phe-Bta-Nle-Bpa-Z-], cyclo[-Phe-D-Bta-Nle-Bpa-Z-], cyclo[-Phe-Bta-Nle-B-Nal-Z-], cyclo[-Phe-D-Bta-Nle-1-Nal-Z-], cyclo[-Phe-Bta-Nle-2-Nal-Z-], cyclo[-Phe-D-Bta-Nle-2-Nal-Z-], cyclo[-Phe-Bta-Nle-p-fluoro-Phe-Z-], cyclo[-Phe-D-Bta-Nle-p-fluoro-Phe-Z-], cyclo[-Phe-Bta-Nle-Phe(F5)-Z-], cyclo[-Phe-D-Bta-Nle-Phe(F5)-Z-], cyclo[-Phe-Bta-Nle-o-fluoro-Phe-Z], cyclo[-Phe-D-Bta-Nle-o-fluoro-Phe-Z-], cyclo[-Phe-Bta-Nle-m-fluoro-Phe-Z-], cyclo[-Phe-D-Bta-Nle-m-fluoro-Phe-Z-], cyclo[-Phe-Bta-Nle-Thr(Ar)-Z-], cyclo[-Phe-D-Bta-Nle-Thr(Ar)-Z-], cyclo[-Phe-Bta-Nle-Thr(Bn)-Z-], cyclo[-Phe-D-Bta-Nle-Thr(Bn)-Z-], cyclo[-Phe-Bta-Nle-2,4-difluoro-Phe-Z-], cyclo[-Phe-D-Bta-Nle-2,4-difluoro-Phe-Z-], cyclo[-Phe-Bta-Nle-2,3-difluoro-Phe-Z-], cyclo[-Phe-D-Bta-Nle-2,3-difluoro-Phe-Z-], cyclo[-Phe-Bta-Nle-2,5-difluoro-Phe-Z-], cyclo[-Phe-D-Bta-Nle-2,5-difluoro-Phe-Z-], cyclo[-Phe-Bta-Nle-p-chloro-Phe-Z-], cyclo[-Phe-D-Bta-Nle-p-chloro-Phe-Z-], cyclo[-Phe-Bta-Nle-Phe(C15)-Z-], cyclo[-Phe-D-Bta-Nle-Phe(C15)-Z-], cyclo[-Phe-Bta-Nle-o-chloro-Phe-Z-], cyclo[-Phe-D-Bta-Nle-o-chloro-Phe-Z-], cyclo[-Phe-Bta-Nle-m-chloro-Phe-Z-], cyclo[-Phe-D-Bta-Nle-m-chloro-Phe-Z-], cyclo[-Phe-Bta-Nle-Thr(Ar)-Z-], cyclo[-Phe-Bta-Nle-2,4-dichloro-Phe-Z-], cyclo[-Phe-D-Bta-Nle-2,4-dichloro-Phe-Z-], cyclo[-Phe-Bta-Nle-2,3-dichloro-Phe-Z-], cyclo[-Phe-D-Bta-Nle-2,3-dichloro-Phe-Z-], cyclo[-Phe-Bta-Nle-2,5-dichloro-Phe-Z-], cyclo[-Phe-D-Bta-Nle-2,5-dichloro-Phe-Z-], cyclo[-Phe-Bta-Nle-2,5-dichloro-Phe-Z-], cyclo[-Phe-D-Bta-Nle-2,5-dichloro-Phe-Z-], cyclo[-Phe-Bta-Nle-3,5-dichloro-Phe-Z-], cyclo[-Phe-D-Bta-Nle-3,5-dichloro-Phe-Z-], cyclo[-Phe-Bta-Nle-3,5-difluoro-Phe-Z-], cyclo[-Phe-D-Bta-Nle-3,5-difluoro-Phe-Z-], cyclo[-Phe-Bta-Nle-3,4-dichloro-Phe-Z-], cyclo[-Phe-D-Bta-Nle-3,4-dichloro-Phe-Z-], cyclo[-Phe-Bta-Nle-3,4-difluoro-Phe-Z-], cyclo[-Phe-D-Bta-Nle-3,4-difluoro-Phe-Z-], cyclo[-Phe-Bta-Nle-Nle(6-OBzl)-Z-], cyclo[-Phe-D-Bta-Nle-Nle(6-OBzl)-Z-], cyclo[-Tyr-Bta-Nle-Nle(6-OBzl)-Z-], cyclo[-Tyr-D-Bta-Nle-Nle(6-OBzl)-Z-], cyclo[-Tyr(Me)-Bta-Nle-Nle(6-OBzl)-Z-], cyclo[-Tyr(Me)-D-Bta-Nle-Nle(6-OBzl)-Z-], cyclo[-Phe-Bta-Nle-3-Pal-Z-], cyclo[-Phe-D-Bta-Nle-3-Pal-Z-], cyclo[-Tyr-Bta-Nle-3-Pal-Z-], cyclo[-Tyr-D-Bta-Nle-3-Pal-Z-], cyclo[-Tyr(Me)-Bta-Nle-3-Pal-Z-], cyclo[-Tyr(Me)-D-Bta-Nle-3-Pal-Z-], cyclo[-Phe-Bta-Nle-4-Pal-Z-], cyclo[-Phe-D-Bta-Nle-4-Pal-Z-], cyclo[-Tyr-Bta-Nle-4-Pal-Z-], cyclo[-Tyr-D-Bta-Nle-4-Pal-Z-], cyclo[-Tyr(Me)-Bta-Nle-4-Pal-Z-], cyclo[-Phe-Bta-Nle-3,4-dichloro-Phe-Z-], cyclo[-Phe-D-Bta-Nle-3,4-dichloro-Phe-Z-], cyclo[-Phe-Bta-Nle-3,4-difluoro-Phe-Z-], cyclo[-Phe-D-Bta-Nle-3,4-difluoro-Phe-Z-], cyclo[-Phe-Bta-Nle-Phg-Z-], cyclo[-Phe-D-Bta-Nle-Phg-Z-], cyclo[-Tyr-Bta-Nle-Phg-Z-], cyclo[-Tyr-D-Bta-Nle-Phg-Z-], cyclo[-Tyr(Me)-Bta-Nle-Phg-Z-], cyclo[-Phe-Bta-Nle-hPhe-Z-], cyclo[-Phe-D-Bta-Nle-hPhe-Z-], cyclo[-Tyr-Bta-Nle-hPhe-Z-], cyclo[-Tyr-D-Bta-Nle-hPhe-Z-], cyclo[-Tyr(Me)-Bta-Nle-hPhe-Z-], cyclo[-Phe-Bta-Nle-Igl-Z-], cyclo[-Phe-D-Bta-Nle-Igl-Z-], cyclo[-Tyr-Bta-Nle-Igl-Z-], cyclo[-Tyr-D-Bta-Nle-Igl-Z-], cyclo[-Tyr(Me)-Bta-Nle-Igl-Z-], cyclo[-Phe-Bta-Nle-Phe(4-NO2)-Z-], cyclo[-Phe-D-Bta-Nle-Phe(4-NO2)-Z-], cyclo[-Tyr-Bta-Nle-Phe(4-NO2)-Z-], cyclo[-Tyr-D-Bta-Nle-Phe(4-NO2)-Z-], cyclo[-Tyr(Me)-Bta-Nle-Phe(4-NO2)-Z-], cyclo[-Phe-Bta-Nle-Phe(4-NHz)-Z-], cyclo[-Phe-D-Bta-Nle-Phe(4-NHz)-Z-], cyclo[-Tyr-Bta-Nle-Phe(4-NHz)-Z-], cyclo[-Tyr-D-Bta-Nle-Phe(4-NHz)-Z-], cyclo[-Tyr(Me)-Bta-Nle-Phe(4-NHz)-Z-], cyclo[-Phe-Bta-Nle-Phe(4-NH-2Clz)-Z-], cyclo[-Phe-D-Bta-Nle-Phe(4-NH-2Clz)-Z-], cyclo[-Tyr-Bta-Nle-Phe(4-NH-2Clz)-Z-], cyclo[-Tyr-D-Bta-Nle-Phe(4-NH-2Clz)-Z-], cyclo[-Tyr(Me)-Bta-Nle-Phe(4-NH-2Clz)-Z-], cyclo[-Phe-Bta-Nle-hTyr-Z-], cyclo[-Phe-D-Bta-Nle-hTyr-Z-], cyclo[-Tyr-Bta-Nle-hTyr-Z-], cyclo[-Tyr-D-Bta-Nle-hTyr-Z-], cyclo[-Tyr(Me)-Bta-Nle-hTyr-Z-], cyclo[-Phe-Bta-Nle-Pra-Z-], cyclo[-Phe-D-Bta-Nle-Pra-Z-], cyclo[-Tyr-Bta-Nle-Pra-Z-], cyclo[-Tyr-D-Bta-Nle-Pra-Z], cyclo[-Phe-Trp-Lys-Nle(6-OBzl)-Z-], cyclo[-Phe-D-Trp-Lys-Nle(6-OBzl)-Z-], cyclo[-Tyr-Trp-Lys-Nle(6-OBzl)-Z-], cyclo[-Tyr-D-Trp Lys-Nle(6-OBzl)-Z-], cyclo[-Tyr(Me)-Trp-Lys-Nle(6-OBzl)-Z-], cyclo[-Phe-Trp-Lys-3-Pal-Z-], cyclo[-Phe-D-Trp-Lys-3-Pal-Z-], cyclo[-Tyr-Trp-Lys-3-Pal-Z-], cyclo[-Tyr-D-Trp-Lys-3-Pal-Z-], cyclo[-Tyr(Me)-Trp-Lys-3-Pal-Z-], cyclo[-Tyr(Me)-D-Trp-Lys-3-Pal-Z-], cyclo[-Phe-Trp-Lys-4-Pal-Z-], cyclo[-Phe-D-Trp-Lys-4-Pal-Z-], cyclo[-Tyr-Trp-Lys-4-Pal-Z-], cyclo[-Tyr-D-Trp-Lys-4-Pal-Z-], cyclo[-Tyr(Me)-Trp-Lys-4-Pal-Z-], cyclo[-Phe-Trp-Lys-3,4-dichloro-Phe-Z-], cyclo[-Phe-D-Trp-Lys-3,4-dichloro-Phe-Z-], cyclo[-Phe-Trp-Lys-3,4-difluoro-Phe-Z-], cyclo[-Phe-D-Trp-Lys-3,4-difluoro-Phe-Z-], cyclo[-Phe-Trp-Lys-Phg-Z-], cyclo[-Phe-D-Trp-Lys-Phg-Z-], cyclo[-Tyr-Trp-Lys-Phg-Z-], cyclo[-Tyr-D-Trp-Lys-Phg-Z-], cyclo[-Tyr(Me)-Trp-Lys-Phg-Z-], cyclo[-Phe-Trp-Lys-hPhe-Z-], cyclo[-Phe-D-Trp-Lys-hPhe-Z-], cyclo[-Tyr-Trp-Lys-hPhe-Z-], cyclo[-Tyr-D-Trp-Lys-hPhe-Z-], cyclo[-Tyr(Me)-Trp,-Lys-hPhe-Z-], cyclo[-Phe-Trp-Lys-Igl-Z-], cyclo[-Phe-D-Trp-Lys-Igl-Z-], cyclo[-Tyr-Trp-Lys-Igl-Z-], cyclo[-Tyr-D-Trp-Lys-Igl-Z-], cyclo[-Tyr(Me)-Trp-Lys-Igl-Z-], cyclo[-Phe-Trp-Lys-Phe(4-NO2)-Z-], cyclo[-Phe-D-Trp-Lys-Phe(4-NO2)-Z-], cyclo[-Tyr-Trp-Lys-Phe(4-NO2)-Z-], cyclo[-Tyr-D-Trp-Lys-Phe(4-NO2)-Z-], cyclo[-Tyr(Me)-Trp-Lys-Phe(4-NO2)-Z-], cyclo[-Phe-Trp-Lys-Phe(4-NHz)-Z-], cyclo[-Phe-D-Trp-Lys-Phe(4-NHz)-Z-], cyclo[-Tyr-Trp-Lys-Phe(4-NHz)-Z-], cyclo[-Tyr-D-Trp-Lys-Phe(4-NHz)-Z-], cyclo[-Tyr(Me)-Trp-Lys-Phe(4-NHz)-Z-], cyclo[-Phe-Trp-Lys-Phe(4-NH-2Clz)-Z-], cyclo[-Phe-D-Trp-Lys-Phe(4-NH-2Clz)-Z-], cyclo[-Tyr-Trp-Lys-Phe(4-NH-2Clz)-Z-], cyclo[-Tyr-D-Trp-Lys-Phe(4-NH-2Clz)-Z-], cyclo[-Tyr(Me)-Trp-Lys-Phe(4-NH-2Clz)-Z-], cyclo[-Phe-Trp-Lys-hTyr-Z-], cyclo[-Phe-D-Trp-Lys-hTyr-Z-], cyclo[-Tyr-Trp-Lys-hTyr-Z-], cyclo[-Tyr-D-Trp-Lys-hTyr-Z-], cyclo[-Tyr(Me)-Trp-Lys-hTyr-Z-], cyclo[-Phe-Trp-Lys-Pra-Z-], cyclo[-Phe-D-Trp-Lys-Pra-Z-], cyclo[-Tyr-Trp-Lys-Pra-Z-], cyclo[-Tyr-D-Trp-Lys-Pra-Z-], cyclo[-Tyr(Me)-Trp-Lys-Pra-Z-], cyclo[-Phe-1-Nal-Lys-Phe-Z-], cyclo[-Phe-D-1-Nal-Lys-Phe-Z-], cyclo[-Tyr-1-Nal-Lys-Phe-Z-], cyclo[-Tyr-D-1-Nal-Lys-Phe-Z-], cyclo[-Tyr(Me)-1-Nal-Lys-Phe-Z-], cyclo[-Tyr(Me)-D-1-Nal-Lys-Phe-Z-], cyclo[-Phe-1-Nal-Lys-Thr-Z-], cyclo[-Phe-D-1-Nal-Lys-Thr-Z-], cyclo[-Phe-1-Nal-Lys-Tyr(Bzl)-Z-], cyclo[-Phe-D-1-Nal-Lys-Tyr(Bzl)-Z-], cyclo[-Phe-1-Nal-Lys-Bip-Z-], cyclo[-Phe-D-1-Nal-Lys-Bip-Z-], cyclo[-Phe-1-Nal-Lys-Dip-Z-], cyclo[-Phe-D-1-Nal-Lys-Dip-Z-], cyclo[-Phe-1-Nal-Lys-Bpa-Z-], cyclo[-Phe-D-1-Nal-Lys-Bpa-Z-], cyclo[-Phe-1-Nal-Lys-1-Nal-Z-], cyclo[-Phe-D-1-Nal-Lys-1-Nal-Z-], cyclo[-Phe-1-Nal-Lys-2-Nal-Z-], cyclo[-Phe-D-1-Nal-Lys-2-Nal-Z-], cyclo[-Phe-1-Nal-Lys-p-fluoro-Phe-Z-], cyclo[-Phe-D-1-Nal-Lys-p-fluoro-Phe-Z-], cyclo[-Phe-1-Nal-Lys-Phe(F5)-Z-], cyclo[-Phe-D-1-Nal-Lys-Phe(F5)-Z-], cyclo[-Phe-1-Nal-Lys-o-fluoro-Phe-Z-], cyclo[-Phe-D-1-Nal-Lys-o-fluoro-Phe-Z-], cyclo[-Phe-1-Nal-Lys-m-fluoro-Phe-Z-], cyclo[-Phe-D-1-Nal-Lys-m-fluoro-Phe-Z-], cyclo[-Phe-1-Nal-Lys-Thr(Ar)-Z-], cyclo[-Phe-D-1-Nal-Lys-Thr(Ar)-Z-], cyclo[-Phe-1-Nal-Lys-Thr(Bn)-Z-], cyclo[-Phe-D-1-Nal-Lys-Thr(Bn)-Z-], cyclo[-Phe-1-Nal-Lys-2,4-difluoro-Phe-Z-], cyclo[-Phe-D-1-Nal-Lys-2,4-difluoro-Phe-Z-], cyclo[-Phe-1-Nal-Lys-2,3-difluoro-Phe-Z-], cyclo[-Phe-D-1-Nal-Lys-2,3-difluoro-Phe-Z-], cyclo[-Phe-1-Nal-Lys-2,5-difluoro-Phe-Z-], cyclo[-Phe-D-1-Nal-Lys-2,5-difluoro-Phe-Z-], cyclo[-Phe-1-Nal-Lys-p-chloro-Phe-Z-], cyclo[-Phe-D-1-Nal-Lys-p-chloro-Phe-Z-], cyclo[-Phe-1-Nal-Lys-Phe(C15)-Z-], cyclo[-Phe-D-1-Nal-Lys-Phe(C15)-Z-], cyclo[-Phe-1-Nal-Lys-o-chloro-Phe-Z-], cyclo[-Phe-D-1-Nal-Lys-o-chloro-Phe-Z-], cyclo[-Phe-1-Nal-Lys-m-chloro-Phe-Z-], cyclo[-Phe-D-1-Nal-Lys-m-chloro-Phe-Z-], cyclo[-Phe-1-Nal-Lys-Thr(Ar)-Z-], cyclo[-Phe-1-Nal-Lys-2,4-dichloro-Phe-Z-], cyclo[-Phe-D-1-Na-Lys-2,4-dichloro-Phe-Z-], cyclo[-Phe-1-Nal-Lys-2,3-dichloro-Phe -Z-], cyclo[-Phe-D-1-Nal-Lys -2,3-dichloro-Phe-Z-], cyclo[-Phe-1-Nal-Lys-2,5-dichloro-Phe-Z-], cyclo[Phe-D-1-Nal-Lys -2,5-dichloro-Phe-Z-], cyclo[-Phe-1-Nal -Lys-2,5-dichloro-Phe-Z-], cyclo[-Phe-D-1-Nal-Lys-2,5-dichloro-Phe-Z-], cyclo[-Phe-1-Nal -Lys-3,5-dichloro-Phe-Z-], cyclo[-Phe-D -1-Nal-Lys-3,5-dichloro-Phe -Z-], cyclo[-Phe-1-Nal-Lys-3,5-difluoro-Phe-Z-], cyclo[-Phe-D-1-Nal-Lys-3,5-difluoro-Phe-Z-], cyclo[-Phe-1-Nal-Lys-Nle(6-OBzl)-Z-], cyclo[-Phe-D-1-Nal-Lys-Nle(6-OBzl)-Z-], cyclo[-Tyr-1-Nal-Lys-Nle(6-OBzl)-Z-], cyclo[-Tyr-D-1-Nal-Lys-Nle(6-OBzl)-Z-], cyclo[-Tyr(Me)-1-Nal-Lys-Nle(6-OBzl)-Z-], cyclo[-Tyr(Me)-D-1-Nal-Lys-Nle(6-OBzl)-Z-], cyclo[-Phe-1-Nal-Lys-3,4-dichloro-Phe-Z-], cyclo[-Phe-D-1-Nal-Lys-3,4-dichloro-Phe-Z-], cyclo[-Phe-1-Nal-Lys-3,4-difluoro-Phe-Z-], cyclo[-Phe-D-1-Nal-Lys-3,4-difluoro-Phe-Z-], cyclo[-Phe-1-Nal-Lys-3-Pal-Z-], cyclo[-Phe-D-1-Nal-Lys-3-Pal-Z-], cyclo[-Tyr-1-Nal-Lys-3-Pal-Z-], cyclo[-Tyr-D-1-Nal-Lys-3-Pal-Z-], cyclo[-Tyr(Me)-1-Nal-Lys-3-Pal-Z-], cyclo[-Tyr(Me)-D-1-Nal-Lys-3-Pal-Z-], cyclo[-Phe-1-Nal-Lys-4-Pal-Z-], cyclo[-Phe-D-1-Nal-Lys-4-Pal-Z-], cyclo[-Tyr-1-Nal-Lys-4-Pal-Z-], cyclo[-Tyr-D-1-Nal-Lys-4-Pal-Z-], cyclo[-Tyr(Me)-1-Nal-Lys-4-Pal-Z-], cyclo[-Phe-1-Nal-Lys-3,4-dichloro-Phe-Z-], cyclo[-Phe-D-1-Nal-Lys-3,4-dichloro-Phe-Z-], cyclo[-Phe-1-Nal-Lys-3,4-difluoro-Phe-Z-], cyclo[-Phe-D-1-Nal-Lys-3,4-difluoro-Phe-Z-], cyclo[-Phe-1-Nal-Lys-Phg-Z-], cyclo[-Phe-D-1-Nal-Lys-Phg-Z-], cyclo[-Tyr-1-Nal-Lys-Phg-Z-], cyclo[-Tyr-D-1-Nal-Lys-Phg-Z-], cyclo[-Tyr(Me)-1-Nal-Lys-Phg-Z-], cyclo[-Phe-1-Nal-Lys-hPhe-Z-], cyclo[-Phe-D-1-Nal-Lys-hPhe-Z-], cyclo[-Tyr-1-Nal-Lys-hPhe-Z-], cyclo[-Tyr-D-1-Nal-Lys-hPhe-Z-], cyclo[-Tyr(Me)-1-Nal-Lys-hPhe-Z-], cyclo[-Phe-1-Nal-Lys-Igl-Z-], cyclo[-Phe-D-1-Nal-Lys-Igl-Z-], cyclo[-Tyr-1-Nal-Lys-Igl-Z-], cyclo[-Tyr-D-1-Nal-Lys-Igl-Z-], cyclo[-Tyr(Me)-1-Nal-Lys-Igl-Z], cyclo[-Phe-1-Nal-Lys-Phe(4-NO2)-Z-], cyclo[-Phe-D-1-Nal-Lys-Phe(4-NO2)-Z-], cyclo[-Tyr-1-Nal-Lys-Phe(4-NO2)-Z-], cyclo[-Tyr-D-1-Nal-Lys-Phe(4-NO2)-Z-], cyclo[-Tyr(Me)1-Nal-Lys-Phe(4-NO2)-Z-], cyclo[-Phe-1-Nal-Lys-Phe(4-NHz)-Z-], cyclo[-Phe-D-1-Nal-Lys-Phe(4-NHz)-Z-], cyclo[-Tyr-1-Nal-Lys-Phe(4-NHz)-Z-], cyclo[-Tyr-D-1-Nal-Lys-Phe(4-NHz)-Z-], cyclo[-Tyr(Me)-1-Nal-Lys-Phe(4-NHz)-Z-], cyclo[-Phe-1-Nal-Lys-Phe(4-NH-2Clz)-Z-], cyclo[-Phe-D-1-Nal-Lys-Phe(4-NH-2Clz)-Z-], cyclo[-Tyr-1-Nal-Lys-Phe(4-NH-2Clz)-Z-], cyclo[-Tyr-D-1-Nal-Lys-Phe(4-NH-2Clz)-Z-], cyclo[-Tyr(Me)-1-Nal-Lys-Phe(4-NH-2Clz)-Z-], cyclo[-Phe-1-Nal-Lys-hTyr-Z-], cyclo[-Phe-D-1-Nal-Lys-hTyr-Z-], cyclo[-Tyr-1-Nal-Lys-hTyr-Z-], cyclo[-Tyr-D-1-Nal-Lys-hTyr-Z-], cyclo[-Tyr(Me)-1-Nal-Lys-hTyr-Z-], cyclo[-Phe-1-Nal-Lys-Pra-Z-], cyclo[-Phe-D-1-Nal-Lys-Pra-Z-], cyclo[-Tyr-1-Nal-Lys-Pra-Z-], cyclo[-Tyr-D-1-Nal-Lys-Pra-Z-], cyclo[-Tyr(Me)-1-Nal-Lys-Pra-Z-] cyclo[-Phe-2-Nal-Lys-Phe-Z-], cyclo[-Phe-D-2-Nal-Lys-Phe-Z-], cyclo[-Tyr-2-Nal-Lys-Phe-Z-], cyclo[-Tyr-D-2-Nal-Lys-Phe-Z-], cyclo[-Tyr(Me)-2-Nal-Lys-Phe-Z-], cyclo[-Tyr(Me)-D-2-Nal-Lys-Phe-Z-], cyclo[-Phe-2-Nal-Lys-Thr-Z-], cyclo[-Phe-D-2-Nal-Lys-Thr-Z-], cyclo[-Phe-2-Nal-Lys-Tyr(Bzl)-Z-], cyclo[-Phe-D-2-Nal-Lys-Tyr(Bzl)-Z-], cyclo[-Phe-2-Nal-Lys-Bip-Z-], cyclo[-Phe-D-2-Nal-Lys-Bip-Z-], cyclo[-Phe-2-Nal-Lys-Dip-Z-], cyclo[-Phe-D-2-Nal-Lys-Dip-Z-], cyclo[-Phe-2-Nal-Lys-Bpa-Z-], cyclo[-Phe-D-2-Nal-Lys-Bpa-Z-], cyclo[-Phe-2-Nal-Lys-2-Nal-Z-], cyclo[-Phe-D-2-Nal-Lys-2-Nal-Z-], cyclo[-Phe-2-Nal-Lys-1-Nal-Z-], cyclo[Phe-D-2-Nal-Lys-1-Nal-Z-], cyclo[-Phe-2-Nal-Lys-p-fluoro-Phe-Z-], cyclo[-Phe-D-2-Nal-Lys-p-fluoro-Phe-Z-], cyclo[-Phe-2-Nal-Lys-Phe(F5)-Z-], cyclo[-Phe-D-2-Nal-Lys-Phe(F5)-Z-], cyclo[-Phe-2-Nal-Lys-o-fluoro-Phe-Z-], cyclo[-Phe-D-2-Nal-Lys-o-fluoro-Phe-Z-], cyclo[-Phe-2-Nal-Lys-m-fluoro-Phe-Z-], cyclo[-Phe-D-2-Nal-Lys-m-fluoro-Phe-Z-], cyclo[-Phe-2-Nal-Lys-Thr(Ar)-Z-], cyclo[-Phe-D-2-Nal-Lys-Thr(Ar)-Z-], cyclo[-Phe-2-Nal-Lys-Thr(Bn)-Z-], cyclo[-Phe-D-2-Nal-Lys-Thr(Bn)-Z-], cyclo[-Phe-2-Nal-Lys-2,4-difluoro-Phe-Z-], cyclo[-Phe-D-2-Nal-Lys-2,4-difluoro-Phe-Z-], cyclo[-Phe-2-Nal-Lys-2,3-difluoro-Phe-Z-], cyclo[-Phe-D-2-Nal-Lys-2,3-difluoro-Phe-Z-], cyclo[-Phe-2-Nal-Lys-2,5-difluoro-Phe-Z-], cyclo[-Phe-D-2-Nal-Lys-2,5-difluoro-Phe-Z-], cyclo[-Phe-2-Nal-Lys-p-chloro-Phe-Z-], cyclo[-Phe-D-2-Nal-Lys-p-chloro-Phe-Z-], cyclo[-Phe-2-Nal-Lys-Phe(C15)-Z-], cyclo[-Phe-D-2-Nal-Lys-Phe(C15)-Z-], cyclo[-Phe-2-Nal-Lys-o-chloro-Phe-Z-], cyclo[-Phe-D-2-Nal-Lys-o-chloro-Phe-Z-], cyclo[-Phe-2-Nal-Lys-m-chloro-Phe-Z-], cyclo[-Phe-D-2-Nal-Lys-m-chloro-Phe-Z-], cyclo[-Phe-2-Nal-Lys-Thr(Ar)-Z-], cyclo[-Phe-2-Nal-Lys-2,4-dichloro-Phe-Z-], cyclo[-Phe-D-2-Nal-Lys-2,4-dichloro-Phe-Z-], cyclo[-Phe-2-Nal-Lys-2,3-dichloro-Phe-Z-], cyclo[-Phe-D-2-Nal-Lys-2,3-dichloro-Phe-Z-], cyclo[-Phe-2-Nal-Lys-2,5-dichloro-Phe-Z-], cyclo[-Phe-D-2-Nal-Lys-2,5-dichloro-Phe-Z-], cyclo[-Phe-2-Nal-Lys-2,5-dichloro-Phe-Z-], cyclo[-Phe-D-2-Nal-Lys-2,5-dichloro-Phe-Z-], cyclo[-Phe-2-Nal-Lys-3,5-dichloro-Phe-Z-], cyclo[-Phe-D-2-Nal-Lys-3,5-dichloro-Phe-Z-], cyclo[-Phe-2-Nal-Lys-3,5-difluoro-Phe-Z-], cyclo[-Phe-D-2-Nal-Lys-3,5-difluoro-Phe-Z-], cyclo[-Phe-2-Nal-Lys-Nle(6-OBzl)-Z-], cyclo[-Phe-D-2-Nal-Lys-Nle(6-OBzl)-Z-], cyclo[-Tyr-2-Nal-Lys-Nle(6-OBzl)-Z-], cyclo[-Tyr-D-2-Nal-Lys-Nle(6-OBzl)-Z-], cyclo[-Tyr(Me)-2-Nal-Lys-Nle(6-OBzl)-Z-], cyclo[-Tyr(Me)-D-2-Nal-Lys-Nle(6-OBzl)-Z-], cyclo[-Phe-2-Nal-Lys-3,4-dichloro-Phe-Z-], cyclo[-Phe-D-2-Nal-Lys-3,4-dichloro-Phe-Z-], cyclo[-Phe-2-Nal-Lys-3,4-difluoro-Phe-Z-], cyclo[-Phe-D-2-Nal-Lys-3,4-difluoro-Phe-Z-], cyclo[-Phe-2-Nal-Lys-3-Pal-Z-], cyclo[-Phe-D-2-Nal-Lys-3-Pal-Z-], cyclo[-Tyr-2-Nal-Lys-3-Pal-Z-], cyclo[-Tyr-D-2-Nal-Lys-3-Pal-Z-], cyclo[-Tyr(Me)-2-Nal-Lys-3-Pal-Z-], cyclo[-Tyr(Me)-D-2-Nal-Lys-3-Pal -Z-], cyclo[-Phe-2-Nal-Lys-4-Pal-Z-], cyclo[-Phe-D-2-Nal-Lys-4-Pal-Z-], cyclo[-Tyr-2-Nal-Lys-4-Pal-Z-], cyclo[-Tyr-D-2-Nal-Lys-4-Pal-Z-], cyclo[-Tyr(Me)-2-Nal-Lys-4-Pal-Z-], cyclo[-Phe-2-Nal-Lys-3,4-dichloro-Phe-Z-], cyclo[-Phe-D-2-Nal-Lys-3,4-dichloro-Phe-Z-], cyclo[-Phe-2-Nal-Lys-3,4-difluoro-Phe-Z-], cyclo[-Phe-D-2-Nal-Lys-3,4-difluoro-Phe-Z-], cyclo[-Phe-2-Nal-Lys-Phg-Z-], cyclo[-Phe-D-2-Nal-Lys-Phg-Z-], cyclo[-Tyr-2-Nal-Lys-Phg-Z-], cyclo[-Tyr-D-2-Nal-Lys-Phg-Z-], cyclo[-Tyr(Me)-2-Nal-Lys-Phg-Z-], cyclo[-Phe-2-Nal-Lys-hPhe-Z-], cyclo[-Phe-D-2-Nal-Lys-hPhe-Z-], cyclo[-Tyr-2-Nal-Lys-hPhe-Z-], cyclo[-Tyr-D-2-Nal-Lys-hPhe-Z-], cyclo[-Tyr(Me)-2-Nal-Lys-hPhe-Z-], cyclo[-Phe-2-Nal-Lys-Igl-Z-], cyclo[-Phe-D-2-Nal-Lys-Igl-Z-], cyclo[-Tyr-2-Nal-Lys-Igl-Z-], cyclo[-Tyr-D-2-Nal-Lys-Igl-Z-], cyclo[-Tyr(Me)-2-Nal-Lys-Igl-Z-], cyclo[-Phe-2-Nal-Lys-Phe(4-NO2)-Z-], cyclo[-Phe-D-2-Nal-Lys-Phe(4-NO2)-Z-], cyclo[-Tyr-2-Nal-Lys-Phe(4-NO2)-Z-], cyclo[-Tyr-D-2-Nal-Lys-Phe(4-NO2)-Z-], cyclo[-Tyr(Me)-2-Nal-Lys-Phe(4-NO2)-Z-], cyclo[-Phe-2-Nal-Lys-Phe(4-NHz)-Z-], cyclo[-Phe-D-2-Nal-Lys-Phe(4-NHz)-Z-], cyclo[-Tyr-2-Nal-Lys-Phe(4-NHz)-Z-], cyclo[-Tyr-D-2-Nal-Lys-Phe(4-NHz)-Z-], cyclo[-Tyr(Me)-2-Nal-Lys-Phe(4-NHz)-Z-], cyclo[-Phe-2-Nal-Lys-Phe(4-NH-2Clz)-Z-], cyclo[-Phe-D-2-Nal-Lys-Phe(4-NH-2Clz)-Z-], cyclo[-Tyr-2-Nal-Lys-Phe(4-NH-2Clz)-Z-], cyclo[-Tyr-D-2-Nal-Lys-Phe(4-NH-2Clz)-Z-], cyclo[-Tyr(Me)-2-Nal-Lys-Phe(4-NH-2Clz)-Z-], cyclo[-Phe-2-Nal-Lys-hTyr-Z-], cyclo[-Phe-D-2-Nal-Lys-hTyr-Z-], cyclo[-Tyr-2-Nal-Lys-hTyr-Z-], cyclo[-Tyr-D-2-Nal-Lys-hTyr-Z-], cyclo[-Tyr(Me)-2-Nal-Lys-hTyr-Z-], cyclo[-Phe-2-Nal-Lys-Pra-Z-], cyclo[-Phe-D-2-Nal-Lys-Pra-Z-], cyclo[-Tyr-2-Nal-Lys-Pra-Z-], cyclo[-Tyr-D-2-Nal-Lys-Pra-Z-], cyclo[-Tyr(Me)-2-Nal-Lys-Pra-Z-], cyclo[-Phe-Bta-Lys-Phe-Z-], cyclo[-Phe-D-Bta-Lys-Phe-Z-], cyclo[Tyr-Bta-Lys-Phe-Z-], cyclo[-Tyr-D-Bta-Lys-Phe-Z-], cyclo[-Tyr(Me)-Bta-Lys-Phe-Z-], cyclo[-Tyr(Me)-D-Bta-Lys-Phe-Z-], cyclo[-Phe-Bta-Lys-Thr-Z-], cyclo[-Phe-D-Bta-Lys-Thr-Z-], cyclo[-Phe-Bta-Lys-Tyr(Bzl)-Z-], cyclo[-Phe-D-Bta-Lys-Tyr(Bzl)-Z-], cyclo[Phe-Bta-Lys-Bip-Z-], cyclo[-Phe-D-Bta-Lys-Bip-Z-], cyclo[-Phe-Bta-Lys-Dip-Z-], cyclo[-Phe-D-Bta-Lys-Dip-Z-], cyclo[-Phe-Bta-Lys-Bpa-Z-], cyclo[-Phe-D-Bta-Lys-Bpa-Z-], cyclo[-Phe-Bta-Lys-1-Nal-Z-], cyclo[-Phe-D-Bta-Lys-1-Nal-Z-], cyclo[-Phe-Bta-Lys-2-Nal-Z-], cyclo[-Phe-D-Bta-Lys-2-Nal-Z-], cyclo[-Phe-Bta-Lys-p-fluoro-Phe-Z-], cyclo[-Phe-D-Bta-Lys-p-fluoro-Phe-Z-], cyclo[-Phe-Bta-Lys-Phe(F5)-Z-], cyclo[-Phe-D-Bta-Lys-Phe(F5)-Z-], cyclo[-Phe-Bta-Lys-o-fluoro-Phe-Z-], cyclo[-Phe-D-Bta-Lys-o-fluoro-Phe-Z-], cyclo[-Phe-Bta-Lys-m-fluoro-Phe-Z-], cyclo[-Phe-D-Bta-Lys-m-fluoro-Phe-Z-], cyclo[-Phe-Bta-Lys-Thr(Ar)-Z-], cyclo[-Phe-D-Bta-Lys-Thr(Ar)-Z-], cyclo[-Phe-Bta-Lys-Thr(Bn)-Z-], cyclo[-Phe-D-Bta-Lys-Thr(Bn)-Z-], cyclo[-Phe-Bta-Lys-2,4-difluoro-Phe-Z-], cyclo[-Phe-D-Bta-Lys-2,4-difluoro-Phe-Z-], cyclo[-Phe-Bta-Lys-2,3-difluoro-Phe-Z-], cyclo[-Phe-D-Bta-Lys-2,3-difluoro-Phe-Z-], cyclo[-Phe-Bta-Lys-2,5-difluoro-Phe-Z-], cyclo[-Phe-D-Bta-Lys-2,5-difluoro-Phe-Z-], cyclo[-Phe-Bta-Lys-p-chloro-Phe-Z-], cyclo[-Phe-D-Bta-Lys-p-chloro-Phe-Z-], cyclo[-Phe-Bta-Lys-Phe(C15)-Z-], cyclo[-Phe-D-Bta-Lys-Phe(C15)-Z-], cyclo[-Phe-Bta-Lys-o-chloro-Phe-Z-], cyclo[-Phe-D-Bta-Lys-o-chloro-Phe-Z-], cyclo[-Phe-Bta-Lys-m-chloro-Phe-Z-], cyclo[-Phe-D-Bta-Lys-m-chloro-Phe-Z-], cyclo[-Phe-Bta-Lys-Thr(Ar)-Z-], cyclo[-Phe-Bta-Lys-2,4-dichloro-Phe-Z-], cyclo[-Phe-D-Bta-Lys-2,4-dichloro-Phe-Z-], cyclo[-Phe-Bta-Lys-2,3-dichloro-Phe-Z-], cyclo[-Phe-D-Bta-Lys-2,3-dichloro-Phe-Z-], cyclo[-Phe-Bta-Lys-2,5-dichloro-Phe-Z-], cyclo[-Phe-D-Bta-Lys-2,5-dichloro-Phe-Z-], cyclo[-Phe-Bta-Lys-2,5-dichloro-Phe-Z-], cyclo[-Phe-D-Bta-Lys-2,5-dichloro-Phe-Z-], cyclo[-Phe-Bta-Lys-3,5-dichloro-Phe-Z-], cyclo[-Phe-D-Bta-Lys-3,5-dichloro-Phe-Z-], cyclo[-Phe-Bta-Lys-3,5-difluoro-Phe-Z-], cyclo[-Phe-D-Bta-Lys-3,5-difluoro-Phe-Z-], cyclo[-Phe-Bta-Lys-3,4-dichloro-Phe-Z-], cyclo[-Phe-D-Bta-Lys-3,4-dichloro-Phe-Z-], cyclo[-Phe-Bta-Lys-3,4-difluoro-Phe-Z-], cyclo[-Phe-D-Bta-Lys-3,4-difluoro-Phe-Z-], cyclo[-Phe-Bta-Lys-Nle(6-OBzl)-Z-], cyclo[-Phe-D-Bta-Lys-Nle(6-OBzl)-Z-], cyclo[-Tyr-Bta-Lys-Nle(6-OBzl)-Z-], cyclo[-Tyr-D-Bta-Lys-Nle(6-OBzl)-Z-], cyclo[-Tyr(Me)-Bta-Lys-Nle(6-OBzl)-Z-], cyclo[-Tyr(Me)-D-Bta-Lys-Nle(6-OBzl)-Z-], cyclo[-Phe-Bta-Lys-3-Pal-Z-], cyclo[-Phe-D-Bta-Lys-3-Pal-Z-], cyclo[-Tyr-Bta-Lys-3-Pal-Z-], cyclo[-Tyr-D-Bta-Lys-3-Pal-Z], cyclo[-Tyr(Me)-Bta-Lys-3-Pal-Z-], cyclo[-Tyr(Me)-D-Bta-Lys-3-Pal-Z-], cyclo[-Phe-Bta-Lys-4-Pal-Z-], cyclo[-Phe-D-Bta-Lys-4-Pal-Z-], cyclo[-Tyr-Bta-Lys-4-Pal-Z-], cyclo[-Tyr-D-Bta-Lys-4-Pal-Z-], cyclo[-Tyr(Me)-Bta-Lys-4-Pal-Z-], cyclo[-Phe-Bta-Lys-3,4-dichloro-Phe-Z-], cyclo[-Phe-D-Bta-Lys-3,4-dichloro-Phe-Z-], cyclo[-Phe-Bta-Lys-3,4-difluoro-Phe-Z-], cyclo[-Phe-D-Bta-Lys-3,4-difluoro-Phe-Z-], cyclo[-Phe-Bta-Lys-Phg-Z-], cyclo[-Phe-D-Bta-Lys-Phg-Z-], cyclo[-Tyr-Bta-Lys-Phg-Z-], cyclo[-Tyr-D-Bta-Lys-Phg-Z-], cyclo[-Tyr(Me)-Bta-Lys-Phg-Z-], cyclo[-Phe-Bta-Lys-hPhe-Z-], cyclo[-Phe-D-Bta-Lys-hPhe-Z-], cyclo[-Tyr-Bta-Lys-hPhe-Z-], cyclo[-Tyr-D-Bta-Lys-hPhe-Z-], cyclo[-Tyr(Me)-Bta-Lys-hPhe-Z-], cyclo[-Phe-Bta-Lys-Igl-Z-], cyclo[-Phe-D-Bta-Lys-Igl-Z-], cyclo[-Tyr-Bta-Lys-Igl-Z-], cyclo[-Tyr-D-Bta-Lys-Igl-Z-], cyclo[-Tyr(Me)-Bta-Lys-Igl-Z-], cyclo[-Phe-Bta-Lys-Phe(4-NO2)-Z-], cyclo[-Phe-D-Bta-Lys-Phe(4-NO2)-Z-], cyclo[-Tyr-Bta-Lys-Phe(4-No2)-Z-], cyclo[-Tyr-D-Bta-Lys-Phe(4-NO2)-Z-], cyclo[-Tyr(Me)-Bta-Lys-Phe(4-NO2)-Z-], cyclo[-Phe-Bta-Lys-Phe(4-NHz)-Z-], cyclo[-Phe-D-Bta-Lys-Phe(4-NHz)-Z-], cyclo[-Tyr-Bta-Lys-Phe(4-NHz)-Z-], cyclo[-Tyr-D-Bta-Lys-Phe(4-NHz)-Z-], cyclo[-Tyr(Me)-Bta-Lys-Phe(4-NHz)-Z-], cyclo[-Phe-Bta-Lys-Phe(4-NH-2Clz)-Z-], cyclo[-Phe-D-Bta-Lys-Phe(4-NH-2Clz)-Z-], cyclo[-Tyr-Bta-Lys-Phe(4-NH-2Clz)-Z-], cyclo[-Tyr-D-Bta-Lys-Phe(4-NH-2Clz)-Z-], cyclo[-Tyr(Me)-Bta-Lys-Phe(4-NH-2Clz)-Z-], cyclo[-Phe-Bta-Lys-hTyr-Z-], cyclo[-Phe-D-Bta-Lys-hTyr-Z-], cyclo[-Tyr-Bta-Lys-hTyr-Z-], cyclo[-Tyr-D-Bta-Lys-hTyr-Z-], cyclo[-Tyr(Me)-Bta-Lys-hTyr-Z-], cyclo[-Phe-Bta-Lys-Pra-Z-], cyclo[-Phe-D-Bta-Lys-Pra-Z-], cyclo[-Tyr-Bta-Lys-Pra-Z-], cyclo[-Tyr-D-Bta-Lys-Pra-Z], cyclo[-Phe-D-Trp-Nle-Tyr(Bzl)-Z-], cyclo[-Phe-Trp-Nle-Tyr(Bzl)-Z-], cyclo[-Tyr-D-Trp-Nle-Tyr(Bzl)-Z-], cyclo[-Tyr-Trp-Nle-Tyr(Bzl)-Z-], cyclo[-Phe-D-Bta-Nle-Tyr(Bzl)-Z-], cyclo[-Phe-Bta-Nle-Tyr(Bzl)-Z-], cyclo[-Tyr-D-Bta-Nle-Tyr(Bzl)-Z-], cyclo[-Tyr-Btqa-Nle-Tyr(Bzl)-Z-], and cyclo[-Tyr(Me)-Bta-Lys-Pra-Z-].

Also preferred are all the linear peptides which may be derived by replacing a peptide linkage in the above-mentioned sequences with the terminal groups y¹ and y².

A few representatives of the peptides of the invention are graphically shown in the following:

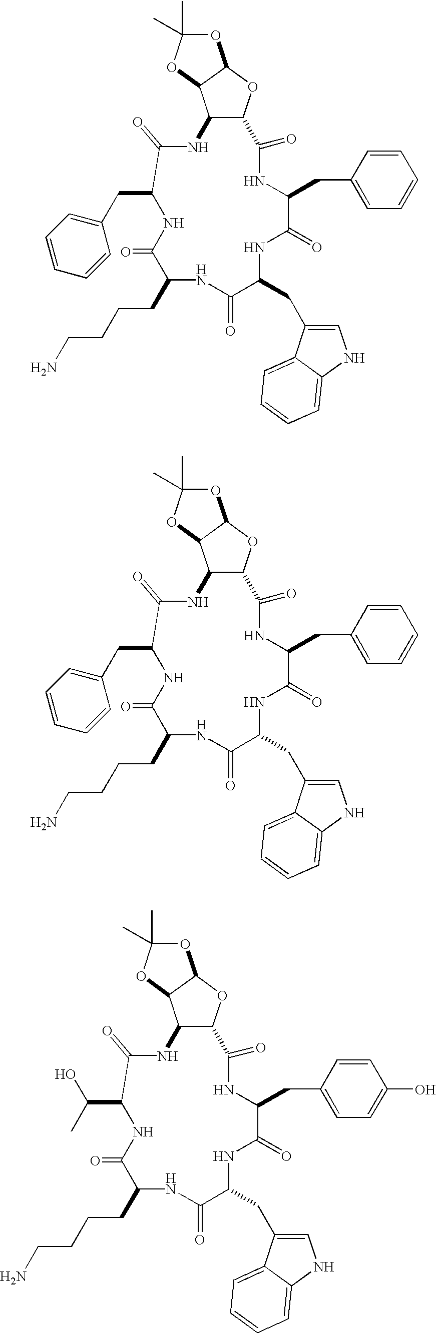

Preferred peptides are shown below:

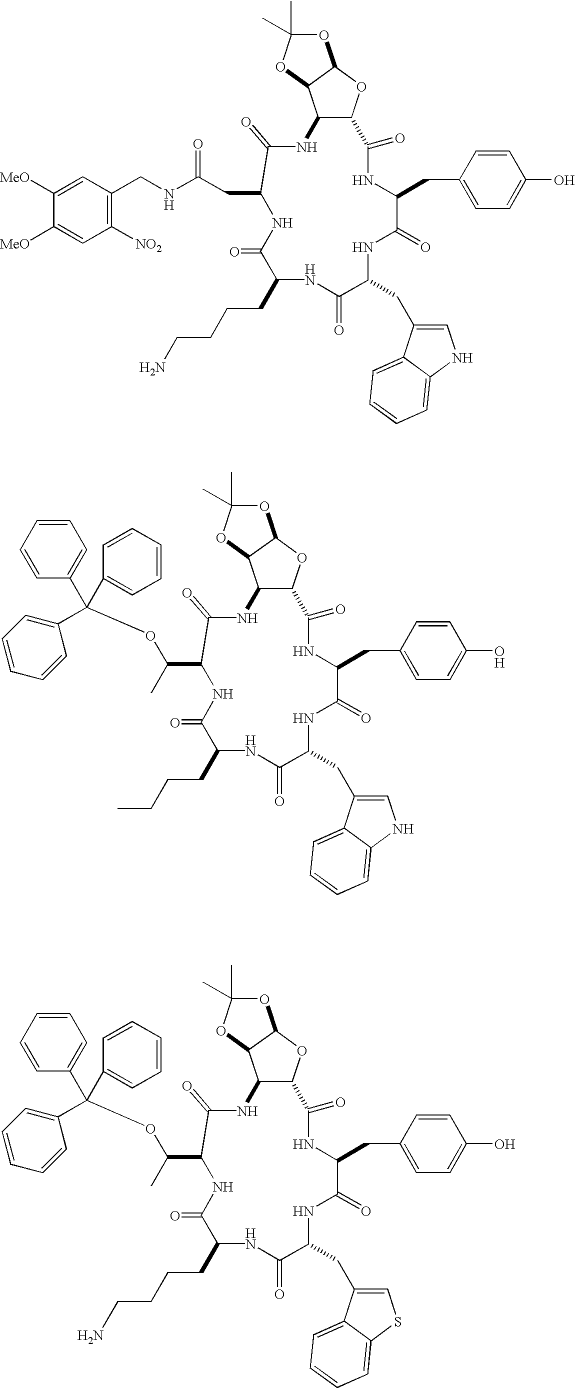

The following peptides are especially preferred:

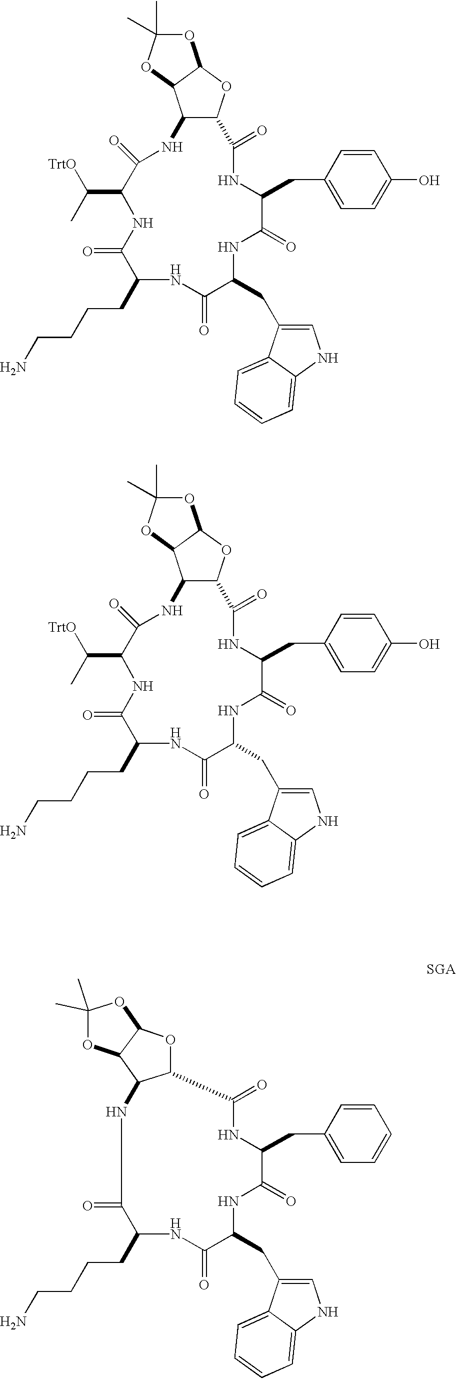

3. Production of the Somatostatin Derivatives of the Invention

The general synthesis of the Z groups and of the peptide of the invention are described below.

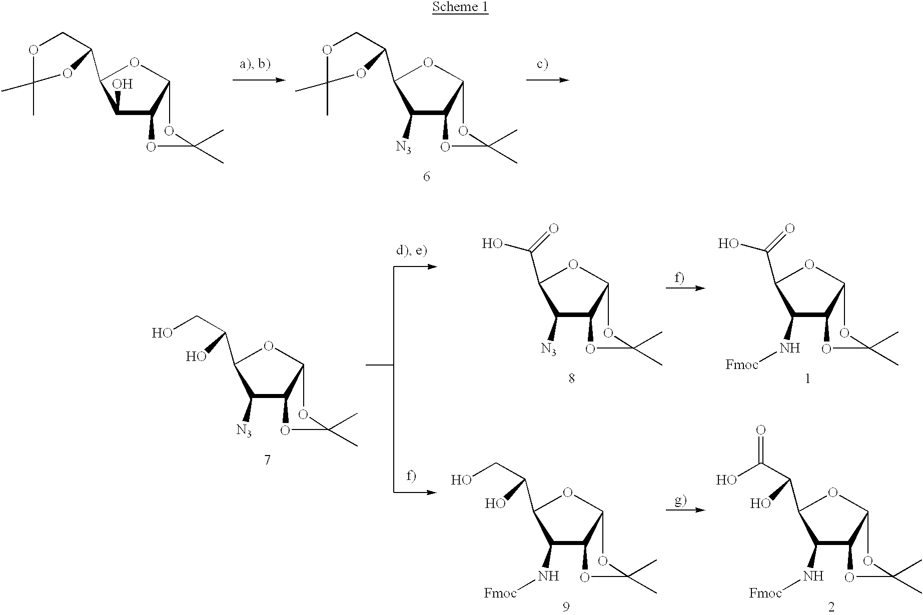

Synthesis of the Fmoc-protected groups Z1 and Z2:

a) Tf₂O, py, −10° C., CH₂Cl₂; b) NaN₃, Bu₄NCl (cat), 50° C., DMF; c) 77% HOAc, 3 h, 65° C.; d) NaIO₄, 5 h, 10° C., MeOH; e) KMnO₄, 50% HOAc, rt; f) H₂, Pd/C, MeOH, FmocCl, NaHCO₃, pH 8-9, THF, MeOH, rt, 90%; g) NaOCl, TEMPO (cat), KBr, CH₂Cl₂, sat. aq NaHCO₃, Bu₄NCl, 62%.

Scheme 1 shows the synthesis of two Fmoc-protected Z groups (1 and 2). Both are synthesised using the azides 6 and 7. The decisive step is acidolysis of diacetone glucose activated over triflate ester. The use of NaN₃ and of catalytic amounts of tetrabutylammonium chloride (Bu₄NCl) is preferred. The azide 6 may be obtained after 3 to 5 hours by reacting triflyl-activated diacetone glucose with 1.8 to 2.5, preferably 1.8 to 2.2 equivalents of NaN₃ in DMF at 30 to 90° C., preferably 40 to 60° C. The use of two equivalents at 50° C. yields optimum results. Catalytic amounts of Bu₄NCl are used to suppress the elimination reaction and to increase the solubiltiy of NaN₃. This affords yields of about 70%.

Azidolysis is followed by deprotection of the exocyclic hydroxyl groups. This may be carried out at quantitative yields by means of acetic acid at a temperature of 20 to 120° C., preferably 70 to 115° C. ((L. N. Kulinkovich, V. A. Timoshchuk, Zh. Obshch. Khim. (RU); 53; 9, 1983; 2126-2131 1983, 53, 1917).

In order to obtain the Fmoc-protected compound 1, the diol 7 is cleaved oxidatively with NaIO₄ and then KMnO₄. These reagents are used in a relative amount of 1.1 to 2.5, preferably 1.5 to 2.2. Suitable reaction temperatures are in the range of 10 to 30° C., preferably 20 to 25° C.

In a one-pot reaction, the-azide 8 is simultaneously reduced with a yield of 70% and Fmoc-protected to obtain 1. With stirring, a solution of the azide in MeOH/H₂O (2:1, 0,15 mol/l) is adjusted to a pH of 8 with saturated NaHCO₃. For this purpose, a solution of Fmoc-Cl (1.0 bis 1.5 equiv., preferably 1.1 equiv.) in THF (0.1 bis 0.2 mol/l, preferably 0.16 mol/l) is added, followed by the addition of the catalyst (Pd/C, 10 wt.-%, wet 49.7 wt-.% H₂O, eg. Degussa E 101; 1 g of catalyst per 1 g of azide). The suspension is washed with H₂ several times. In general, the reaction is completed in 18 to 24 hrs. (control via thin-layer chromatography). The solvents are removed at reduced pressure. The residue is suspended in water and adjusted to a pH of 8 to 9 with saturated NaHCO₃ and the aqueous phase extracted three times with ethyl acetate. The combined organic phases are washed three times with aqueous NaHCO₃ solution. The aqueous phase is adjusted to a pH of 1 with mol/l HCl and then extracted three times with ethyl acetate. The combined organic phases are washed with a saturated aqueous NaCl solution dried over MgSO₄ and concentrated under reduced pressure.

In order to prepare 2, the azide 7 is reduced in a one-pot reaction under similar conditions as for 8 and Fmoc-protected.

After that, the primary alcohol of the product 9 is selectively oxidised with 2,2,6,6-tetramethylpiperidine-1-oxyl (TEMPO), sodium hypochlorite und KBr to yield 2. For this purpose, relative amounts of 0.005 to 0.2 parts of TEMPO, 1 to 5 parts of sodium hypochlorite and 0.5 to 5 KBr, in each case based on 100 mol equivalents of compound 9, are suitable. In order to avoid decarboxylation during oxidation, it is essential to maintain the pH between 8.5 and 9.5 and the temperature below 0° C. Preferred reaction temperatures are in the range of −10 to 0° C.

Other Z groups may be prepared by the following methods described in literature: T. K. Chakraborty, S. Gosh, S. Jayaprakash, J. A. R. P. Sharma, V. Ravikanth, P. V. Diwan, R. Nagaraj, A. C. Kunwar, J. Org. Chem. 2000, 65; M. D. Smith, D. D. Long, A. Martin, D. G. Marquess, T. D. E. Claridge, G. W. J. Fleet, Tetrahedron Lett. 1999, 40, 2191; T. D. W. Claridge, D. D. Long, N. L. Hungerford, R. T. Aplin, M. D. Smith, D. G. Marquess, G. W. J. Fleet, Tetrahedron Lett. 1999, 40, 2199; M. Shiozaki, N. Ishida, S. Sato, Bull. Chem. Soc. Jpn. 1989, 62, 3950.

In addition, suitable Z groups may also be prepared according to WO 95/07022 A, EP 0 538 691 A, EP 0 538 692 A, Yaoxue Xuebao 1985, 20(3), 214-218; J. Nat. Sci. Math. 1983, 23(1), 107-112; Russ. J. Bioorg. Chem. 2000, 26(11), 774-783; Phytochemistry 2000, 53(2), 231-237; Left. Pept. Sci. 1995, 2(3/4), 253-258; JP 46025379 B; Seikagaku 1968, 40(11), 823-837; Liver Res., trans. Int. Symp. 3rd, Tokyo, Kyoto 1967, Meeting Date 1966, 321-330; J. Chem. 1967, 20(12), 2701-2713; Aust. J. Chem. 1967, 20(7), 1493-1509; Nippon Yakuzaishikai Zasshi 1966, 62, 297-306; Hsueh Pao [Acta Pharmaceutica Sinica] 1985, 20(3), 214-218.

Peptide synthesis is carried out according to standard procedures on the solid phase or in solution. Reference is made to G. B. Fields, R. L. Nobel, Int. J. Pept. Protein Res. 1990, 35, 161-214 and to the following general operating instructions “Beladung von TCP-Harz” (loading of TCP resin) and “Abspaltbedingungen für Peptide von TCP-Harz” (cleaving conditions for peptides of TCP resins), form sheets by PepChem, Goldhammer & Clausen, Im Winkelrain 73, D-72076 Tübingen, Germany; Fax ++ 49 70 71 600 393; Tel.: ++ 49-7071-600384; Novabiochem Catalog 2000: “Useful information, Nomenclature, Abbreviations” pages x-xi. and “Synthesis notes” edited by B. Dörner & P. White; pages i-ii, I1-I16, S1-S54, P1-P34, B1-B16, R1-R16, Al-16 Calbiochem-Novabiochem GmbH, P.O Box 1167, 65796 Bad Soden; Tel.: 0800-6931000 or 06196-63955; Fax: ++49-6196-62361. Reference is also made to Solid-Phase Synth. 2000, 377-418 and to R. Knorr, A. Trzeciak, W. Bannwarth, D. Gillessen, Tetrahedron Lett. 1989, 30, 1927-1930. The use of the reagents HATU/HOAt is described in L. A. Carpino, A. El-Faham, F. Albericio, Tetrahedron Lett. 1994, 35, 2279-2282 and in L. A. Carpino, A. El-Faham, C. A. Minor, F. Albericio, J. Chem. Soc. Chem. Commun. 1994, 2, 201-203. The cleavage with HFIP is disclosed in R. Bollhagen, M. Schmiedberger, K. Barlos, E. Grell, J. Chem. Soc., Chem. Commun. 1994, 22, 2559-2560 and the use of the ivDde-protecting group is described in S. R. Chhabra, B. Hothi, D. J. Evans, P. D. White, B. W. Bycroft, W. C. Chan, Tetrahedron Lett. 1998, 39, 1603-1606. The cyclization with DPPA is described in T. Shioiri, K. Ninomiya, S. Yamada, J. Am. Chem. Soc. 1972, 94, 6203-6205 and in S. F. Brady, W. J. Paleveda, B. H. Arison, R. M. Freidinger, R. F. Nutt, D. F. Veber, in 8th Am. Pept. Symp. (Eds.: V. J. Hruby, D. H. Rich), Pierce Chem. Co., Rockford, Ill., USA, Tuscon, Ariz., USA, 1983, pp. 127-130.

4. The use of Somatostatin Derivatives as Anti-Tumour Agents

The application of the peptides of the invention as anti-tumour agents is made in accordance with standard methods known to skilled practitioners from the prior art. Among others, such applications include the use of the peptide of the invention together with the usual, pharmaceutically acceptable excipients and/or the usual pharmaceutically acceptable carriers for preparing a pharmaceutical composition.

Such pharmaceutical compositions may be used for the therapy of tumours. As a rule, all tumours bearing somatostatin receptors may be treated. Among others, these are tumours of the pituitary gland, mamma carcinomas, glucagonomas, renal carcinomas, prostate carcinomas, meningiomas, gliomas, pancreas tumours, insulinomas and liver tumours.

The treatment of the tumours is also carried out in accordance with standard procedures.

5. The use of the Somatostatin Derivatives as Diagnostic Agents for Tumours

Methods for tumour diagnosis by means of positron-emission tomography (PET) and radioscintigraphie as well as other radiodiagnostic methods are known to skilled practitioners from the prior art. This also applies for the radionuclides to be used for this purpose and their suitable complexing agents and bifuntional chelators [Chemical Reviews thematic issue: Medicinal Inorganic Chemistry; September 1999 Volume 99, No. 9; Guest Editors: Chris Orvig, University of British Columbia; Michael J. Abrams, AnorMED, Inc.]. By way of example, reference is made to the following four publications describing the use of the ¹⁸F isotope for tumour diagnosis (R. Haubner, H.-J. Wester, W. Weber, C. Mang, S. Ziegler, R. Senekowitsch-Schmidtke, H. Kessler, M. Schwaiger, Cancer Research 2000, 61, 1781), and of the ¹²⁵I-isotope (R. Haubner, H.-J. Wester, U. Reuning, R. Senekowitsch-Schmidtke, B. Diefenbach, H. Kessler, G. Stöcklin, M. Schwaiger, J. Nucl. Med. 1999, 40, 1061), and of that of metallic radioisotopes such as ¹¹¹In and ^{99m}Tc and suitable bifunctional chelators. Chemical Reviews thematic issue: Medicinal Inorganic Chemistry; September 1999 Volume 99, No. 9 Radiometal-Labeled Agents (Non-Technetium) for Diagnostic Imaging Carolyn J. Anderson and Michael J. Welch pp 2219-2234 and ⁹⁹ mTc-Labeled Small Peptides as Diagnostic Radiopharmaceuticals Shuang Liu and D. Scott Edwardspp 2235-2268.

Thus, the present invention also relates to compounds which are derived from the peptides according to claims 1 to 31, and which contain a radionuclide that is linked to the peptide. Neither the radionuclide to be incorporated into the peptide of the invention nor the method of binding it and its position within the peptide is limited, provided the binding to the somatostatin receptor is not adversely affected and/or the peptide is internalised by tumour cells, so that a signal may be observed with appropriate measurement techniques, that may be used to discriminate the enrichment in tumour tissue from healthy tissue, thereby permitting the diagnosis of tumours. Incorporation of ¹²⁵I and ¹³¹I into the side chain of tyrosine in the radicals A and D is preferred. The incorporation of ^{99m}Tc and ¹¹¹In, ⁶⁷⁶⁸Ga, ^{90/86}Y, ⁶⁴Cu via complexing agents and bifuntional chelators such as DOTA, DTPA (diethylenetriaminepentaacetic acid), EDTA (ethylenediiaminetetraacetic acid), DFO (desferrioxamine-B) or short peptides such as Cys-Gly-Cys, Lys-Gly-Cys or diamidedithiol (DADS) linked to the Z residue are also preferred. The incorporation of ¹²⁵I adjacent to the OH group of tyrosine is particularly preferred.

Structural formulae of suitable chelating groups are as follows:

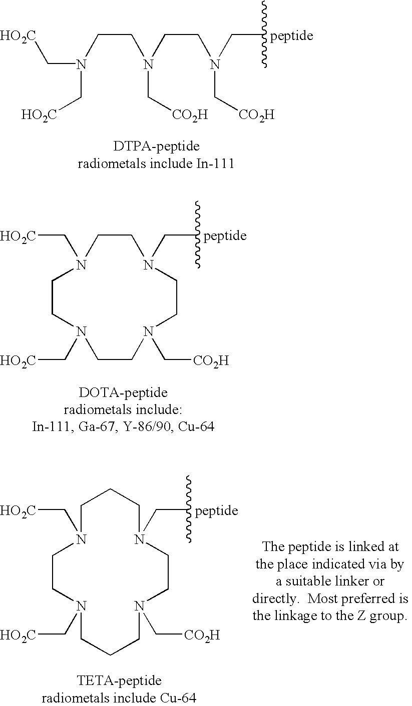

6. The use of the Tetra- and Pentapeptides of the Present Invention as Anti-Inflammatory or Analgetic Agents

This aspect of the present invention is based on the recognition that the development of neurogenic and non-neurogenic inflammations can be prevented and an alleviation of pain can be accomplished by using the compounds of the present invention. Although, as indicated above, somatostatin prevents the experimentally induced neurogenic inflammation, it cannot therapeutically be taken into consideration because of its broad spectrum of activities and its short half life in the human body. Thus the invention relates to the use of tetra- or pentapeptides as described in the claims 1-31 as well as the salts of these compounds for the preparation of pharmaceutical compositions possessing neurogenic or non-neurogenic anti-inflammatory as well as analgetic effects. A common characteristic of the pharmaceutical compostitions prepared by the process of invention is that they inhibit the substance P release (and thus inflammation processes) to a greater extent than natural somatostatin does and in the same range as TT232 does, but they are more stable under the conditions of use. According to the invention, pharmaceutical compositions useful for the inhibition of neurogenic and non-neurogenic inflammations and for pain alleviation can be prepared by mixing the compounds of claims 1-31, the salts or metal complexes thereof with carriers and/or auxiliaries commonly used in the pharmaceutical industry, thereby transforming them into pharmaceutical compositions. The pharmaceutical composition for the therapeutic use may contain any solvent suitable for pharmaceutical use (e.g. water, aqueous solution containing thioalcohol and/or polyalcohol such as polyethylene glycol and/or glycerol etc.); salts (e.g. sodium chloride for adjustment of the physiological osmotic pressure; iron cobalt, zinc or copper chlorides and the like for supplementing trace elements); fillers and carriers (e.g. lactose, potato starch, talc, magnesium carbonate, calcium carbonate, waxes, vegetable oils, polyalcohols etc.); auxiliaries promoting dissolution (such as certain polar solvents, in the case of water usually ethanol, polyalcohols, most frequently polyethylene glycol or glycerol and/or complex forming agents, e.g. cyclodextrins, crown ethers, natural proteins, saponins and the like); tablet-disintegrating agents (artificial or natural polymers strongly swelling in water, e.g. carboxymethylcellulose); complex-forming agents usually employed in retard compositions (such as water-insolble or slightly soluble cyclodextrin derivatives, artificial and natural polymers, crown ethers and the like); pH-adjusting compounds such as mineral or organic buffers; taste-improving agents (cyclodextrins and/or crown ethers); and flavouring agents (beet sugar, fruit sugar or grape sugar, saccharin, invert sugar etc.); antioxidants (e.g. vitamin C) as well as substances promoting the effectiveness of the action of compounds of claims 1-31.

The compounds of claims 1-31 are useful also in aerosol compositions aimed at the absorption through the skin surface or lungs.

For the preparation of tablets, dragées or hard gelatine capsules e.g. lactose, maize, wheat or potato starches, talc, magnesium carbonate, stearic acid and its salts etc. can be used as carriers. For the preparation of soft gelatine capsules e.g. vegetable oils, fats, waxes, or polyalcohols with an appropriate density can be used as carriers. For the preparation of solutions and syrups e.g. water, polyalcohols such as polyethylene glycol and glycerol, beet sugar, grape sugar, etc. can be employed as carriers. Parenteral compositions may contain water, alcohol, polyalcohols or vegetable oils as carriers. Suppositories may contain e.g. oils, waxes, fats or polyalcohols of appropriate density as carriers.

Suitable doses of the active ingredients can be determined in accordance with standard procedures that are known to the person skilled in the art. Typical doses may be in the range of 0.5 to 5000 μg/kg of body weight. However, higher or lower doses may also be appropriate, depending on the individual case and on the active ingredient that is used.

The main advantages of the invention are as follows:

It allows to diminish inflammations of both neurogenic and non-neurogenic orignin with simultaneous exertion of an analgetic effect.

The somatostatin analogues used in the invention are more slowly decomposed under in vivo conditions than the natural compound; therefore their action is more durable.

### EXAMPLES

General:

All solvents for moisture sensitive reactions were distilled and dried in accordance with standard procedures. The Pd/C used is a donation from Degussa, Frankfurt/Main, Germany. Column chromatographies at increased pressure were carried out with the solvents specified on silica gel 60, 230-400 mesh (Merck KGaA, Darmstadt). Tritylchloropolystyrene resin by PepChem Goldammer & Clausen and HATU by Perseptive Biosystems were used for solid phase syntheses. All reactions in a solution were monitored by means of thin-layer chromatography (0.25 mm precoated silica gel 60 F₂₅₄ aluminium plates; Merck KGaA, Darmstadt). Melting points were measued with a Bütchi-Tottoli apparatus and reported in uncorrected form. Analytical and semi-preparative reverse-phase-HPLC was carried out with the aid of Waters equipment (high pressure pump 510, multi-wavelength detector 490E, chromatography workstation Maxima 820), an apparatus from Beckman (high pressure pump 110B, gradient mixer, controller 420, UV detector Uvicord by Knauer) or a device by Amersham Pharmacia Biotech (Äkta Basic 10/100, autosampler A-900).

The preparative reverse-phase-HPLC was carried out on a Beckman System Gold (high pressure pump module 126, UV detector 166). C₁₈ columns (by-YMC) were used for the chromatographies. The solvents used were A: H₂O+0.1% CF₃COOH and B: CH₃CN+0.1% CF₃COOH. Detection was carried out at 220 and 254 nm.

¹H and ¹³C NMR spectra of the compounds were taken on apparatuses by Bruker, Karlsruhe (Bruker—AC 250, Bruker DMX-500 or Bruker DMX-600). References for the chemical shift of the proton resonances were CHCl₃ (δ=7.24) and DMSO (δ=2.49), respectively. Multiplets were noted as s (singlet), d (doublet), t (triplet), q (quartet), m (multiplet), and br (broad). The chemical shift for ¹³C resonances is reported in relation to CDCl₃ (δ=77.0) and [D₆] DMSO (δ=39.5), respectively. Die NMR data were processed on a Bruker X32 work station using UXNMR software. The allocation of the proton and carbon signals was carried out by means of HMQC, COSY, TOCSY and HMBC experiments. Where possible, coupling constants were determined from the corresponding 1D-spectra as well as COSY DQF and COSYPE spectra.

HPLC-ESI mass spectra were prepared on a Finnigan device (NCQ-ESI with HPLC conjunction LCQ; HPLC system Hewlett Packard HP 1100; Nucleosil 100 5C₁₈).

IR spectra were recorded on a Perkin-Elmer 257 spectrophotometer.

High-resolution mass spectra were recorded on a Finnigan MAT 95Q with FAB (Cs⁺ ions and m-nitrobenzyl alcohol as Matrix).

In the following experiments, every step is taken at room temperature (18 to 25° C.) unless explicitly specified otherwise.

### Example 1

Preparation of the Z Group

Preparation of the furanoid Z group from diacetone glucose which is available commercially and inexpensively

Both groups Z1 und Z2 are prepared in accordance with the above scheme 1.

1,2:5,6-Di-O-isopropylidene-3-O-triflyl-α-D-glucofuranose: Triflic anhydride (54.2 g, 0.19209 mol) was slowly added with stirring to a solution of diacetone glucose (25 g, 0.96 mol) and pyridine (30.39 g, 0.384 mol) in CH₂Cl₂ (1 l) in a 3-neck flask at −10° C. (acetone-ice cooling bath) (L. D. Hall, D. C. Miller, Carbohydr. Res. 1976, 47, 299; R. W. Binkley, M. G. Ambrose, D. G. Hehemann, J. Org. Chem. 1980, 45, 4387). The pyridinium triflate salt precipitated and the solution turned brown. The reaction was completed after 1.5 hrs. (TLC control: AcOEt/hexane 2:1).

The reaction mixture was added to 1 l of ice water. The aqueous phase was extracted with CH₂Cl₂ (4×). The organic phase was dried with MgSO₄ and distilled several times on a rotatory evaporator while repeatedly adding toluene in order to remove the pyridine from the mixture. The brown residue was extracted with hexane (3×). After removal of the hexane, the desired product was obtained in the form of white crystals (36.88 g, 98%). R_{f}=0.61 (AcOEt/hexane 2:1). Both the melting point and ¹H NMR were congruent with the values given in literature (L. D. Hall, D. C. Miller, Carbohydr. Res. 1976, 47, 299).

3-Azido-3-deoxy-1,2:5,6-di-O-isopropylidene-α-D-allofuranose (6):

A solution of the trifyl sugar described above (37.1 g, 0.0945 mol) dissolved in DMF (200 ml), was slowly added to a solution of NaN₃ (12.3 g, 0.189 mol), Bu₄NCl catalytic, ˜0.1 g) in DMF (1.5 l) at 50° C. After 5 hrs. of stirring at 50° C., the reaction was completed (TLC control: AcOEt/hexane 2:1). The DMF was removed on the rotary evaporator at reduced pressure and the residue dissolved in AcOEt. The organic phase was washed with water (2×). The aqueous phase was re-extracted with AcOEt until no product 6 was detectable by TLC. The combined organic phases were dried over MgSO₄ and the solvent removed. A syrup of 6 and the elimination byproduct was obtained. (¹H NMR showed that the ratio between product and byproduct was 7:3). The crude product 6 was purified by FC (AcOEt/hexane 1.3) and 6 obtained as a colourless liquid (18.2 g, 70%), R_{f}=0.55 (AcOEt/hexane 1:3). The ¹H NMR von 6 was congruent with the values given in literature (H. H. Baer, Y. Gan, Carbohydr. Res. 1991, 210, 233).

3-Azido-3-deoxy-1,2-O-isopropylidene-α-D-allofuranose (7):

For the oxidation step (4), 6 (16 g, 0.056 mol) was dissolved in AcOH (77%, 38 ml) and stirred at reflux for 3 hrs. After removal of the solvent the crude product 7 was purified by FC (AcOEt/hexane 2:1). White crystals of 7 were obtained (10.98 g, 80%).

3-Azido-3-deoxy-1,2-O-isopropylidene-α-D-ribofuranose Aldehyde:

NaIO₄ (8.4 g, 0.036 mmol) was successively added dropwise to a cooled solution (10° C.) of 7 (8 g, 0.0327 mol) in MeOH (60 ml) and H₂O (100 ml) (L. N. Kulinkovich, V. A. Timoshchuk, Zh. Obshch. Khim. (RU); 53; 9; 1983;2126-2131 1983, 53, 1917). The mixture was stirred for 5 hrs. Inorganic salts precipitated after MeOH (150 ml) was added. They were filtered off and washed repeatedly with MeOH. The combined organic phases were concentrated under vacuum on a rotary evaporator until a slightly yellow syrup remained. The aldehyde obtained was used in the oxidation step to obtain 8 without further purifaction.

¹H NMR (250 MHz, CDCl₃/MeOD, 298 K): δ=1.35 (s, CH₃), 1.55 (s, CH₃), 3.65 (dd, J_{3,4}=4.72, J_{2,3}=4.37 Hz, H³), 4.1 (d, J=4.7 Hz, H⁴), 4.7 (dd, J_{1,2}=3.7, J_{2,3}=4.5 Hz, H²), 5.9 (d, J_{1,2}=3.8 Hz, Hl), 9.7 (br. s, H⁵).

3-Azido-3-deoxy-1,2-O-isopropylidene-α-D-ribofuranoic Acid (8):

With stirring, KMnO₄ (6.7 g, 42 mmol) was slowly added to a solution of the aldehyde in HOAc (50%, 150 ml) (L. N. Kulinkovich, V. A. Timoshchuk, Zh. Obshch. Khim. (RU); 53; 9; 1983;2126-2131 1983, 53, 1917), which resulted in a purple solution. After 12 hours, the reaction was completed. The solution was adjusted to a pH of 1 with conc. HCl and excess KMnO₄ removed with Na₂SO₃. The solution was extracted with CHCl₃ (3×). The organic phase was dried with MgSO₄ and the solvent removed under vacuum. Recrystallisation in AcOEt/hexane yielded crystals of 8 (4.29 g, 1.87 mmol, 89% for both steps together).

General Procedure for the Simultaneous Reduction and Protection of the Azides With Fmoc (GP)

With stirring, the solution of the azide in MeOH/H₂O (2:1, 0.15 mol/l) is adjusted to a pH of 8 with saturated NaHCO₃. A solution of Fmoc-Cl (1.1 equiv.) in THF (0.16 mol/l) is added, followed by the addition of the catalyst (Pd/C, 10 wt.-%, (wet) 49.7 wt.-% H₂O, Degussa E 101, 1 g of catalyst per 1 g of azide). The suspension is gassed with H₂ repeatedly. In general, the reaction is completed in 18 to 24 hrs (contol by means of thin-layer chromatography). The solvents are removed under reduced pressure. The solvent is suspended in water and adjusted to a pH of 8-9 with saturated NaHCO₃ and the aqueous phase extracted three times with ethyl acetate. The combined organic phases are washed with aqueous NaHCO₃ solution. The aqueous phase is adjusted to a pH of 1 with 1 mol/l HCl and extracted three times with ethyl acetate. The combined organic phases are washed with a saturated aqueous NaCl solution, dried over MgSO₄ and concentrated under reduced pressure.

3-Amino-3-deoxy-N-9-fluorenylmethoxycarbonyl-1,2-isopropylidene-α-D-ribofuranoic Acid (1):

As described in GP, the azide 8 (1 g, 4.36 mmol) was reduced to the amine and protected with Fmoc at the same time. 1 (1.4 g, 3.29 mmol, 76%) was obtained as a colourless syrup.

¹H NMR (500 MHz, [D6] DMSO, 300 K): δ=1.26 (s, 3H, CH₃), 1.46 (s, 3H, CH₃), 4.07 (m, H³), 4.22 (m, 1H, Fmoc-CH), 4.25 (m, 1H, H⁴), 4.30 (m, 2H, CH₂^{Fmoc}), 4.60 (t, J=4.0, 1H, H²), 5.84 (d, J=3.4, 1H, H¹), 7.32 (m, arom H), 7.40 (m, arom H), 7.63 (m, H^{N}), 7.72 (m, arom H), 7.87 (d, J=7.3 Hz, 2H, arom H); ¹³C NMR (125 MHz, [D₆] DMSO, 300 K): δ=26.06 (CH₃), 26.29 (CH₃), 46.30 (CH^{Fmoc}), 56.25 (C³), 65.61 (CH₂^{Fmoc}), 75.36 (C⁴), 78.01 (C²), 104.17 (C¹), 111.63 (C^{isoprop.}), 119.75 (C^{arom}), 124.89 (C^{arom}), 127.17 (C^{arom}), 143.32 (C⁵); FAB-HRMS calc. C₂₃H₂₃NO₇Na [M+Na]⁺ 448.1372, found: 448.1366.

3-Amino-3-deoxy-N-9-fluorenylmethoxycarbonyl-1,2-isopropylidene-α-D-allofuranose

As described in GP, the azide 7 (2 g, 8.31 mmol) was reduced to the amine and protected with Fmoc at the same time. FC (AcOEt/hexane 1:1) resulted in a white powder of 9 (3.3 g, 7.48 mmol, 92%).

¹H NMR (500 MHz, CDCl₃, 300 K): δ=1.35 (s, 3H, CH₃), 1.55 (s, 3H, CH₃), 2.12 (s, 0.8H, OH), 3.60-4.65 (m, 13H, H², H³, H⁴, H⁵, H⁶, H^{6′}, CH₂^{Fmoc}, CH^{Fmoc}, H₂O), 5.47 (br. s, 1H, H^{N}), 5,80 (br. s, 1H, H¹), 7.32 (m, 2H, H^{arom}), 7.40 (m, 2H, H^{arom}), 7.57 (m, 2H, H^{arom}), 7.76 (d, J=6.7 Hz, 2H, H^{arom}); ¹³C NMR (125 MHz, CDCl₃, 300 K): δ=26.46 (CH₃), 26.61 (CH₃), 47.12 (CH^{Fmoc}), 55.74 (C³), 63.73 (C⁴), 67.47 (C⁵), 79.25 (C²), 80.41 (CH₂^{Fmoc}) 103.77 (C¹), 112.85 (C^{isoprop}), 120.05 (C^{arom}), 124.90 (C^{arom}), 127.80 (C^{arom}), 141.32, 143.53, 143.57 (C^{arom}, C⁶); ESI-MS: calc. C₂₄H₂₇NO₇Na 464.1685, found: 464.1; t_{R}=14.41 (HPLC-MS, 30-90%B in 20 min).

3-Amino-3-deoxy-N-9-fluorenylmetboxycarbonyl-1,2-isopropylidene-α-D-allofuranoic Acid (2):

The diol 9 and TEMPO (1 mg, 0.064 mmol, 0.011 eq) were suspended in CH₂Cl₂ (1.8 ml) at 0° C. A solution of KBr (14.5 mg, 0.064 mmol, 0.11 eq) and tBu₄NCl (8.9 mg) in saturated aq NaHCO₃ was slowly added to the reaction mixture. A mixture of NaOCl (13%, 1.5 ml), saturated NaCl solution (1.32 ml) and saturated NaHCO₃ solution (0.7 ml) was added dropwise to the reaction mixture over 30 min. The reaction mixture was stirred over night and then diluted with AcOEt (2 ml). The organic phase was extracted twice with saturated NaCl solution. The aqueous phase was adjusted to a pH of 2 with 1 N HCl and extracted with AcOEt extrahiert. The solvent was distilled off at reduced pressure, leaving behind a colourless syrup of 2 (0.17 g, 62%).

¹H NMR (500 MHz, [D₆] DMSO, 300 K): δ=1.25 (s, 3H, CH₃), 1.47 (s, 3H, CH₃), 4.05-4.30 (m, 6H, H³, H⁴, H⁵, CH₂^{Fmoc}, CH^{Fmoc}), 4.55 (br. s, 1H, H²), 5.73 (br. s, 1H, H¹), 7.30-7.90 (m, 9H, H^{arom}, H^{N}); ¹³C NMR (125 MHz, [D₆] DMSO, 300 K): δ=23.97 (CH₃), 24.39 (CH₃), 45.19 (CH^{Fmoc}), 51.88 (C³), 63.70 (C⁴), 67.18 (C⁵), 75.30 (C²), 76.98 (CH₂^{Fmoc}) 101.73 (C¹), 111.35 (C^{isoprop.}), 117.20 (C^{arom}), 122.39 (C^{arom}), 124.14 (C^{arom}), 124.51 (C^{arom}), 143.80 (C⁶); FAB-HRMS calc for C₂₄H₂₅NO₈Na [M+Na]⁺ 478.1478, found: 478.14167; t_{R}=15.71 (HPLC-MS, 10-90%B in 20 min).

### Example 2

Parallel Production of TG and TH:

Resin Loading

According to standard methods, TCP resin (1.3 g) was loaded with 629 mg of Fmoc-Tyr-OH, 2.77 ml of collidine in 10 ml of DCM in a 20 ml syringe. The loading was determined to be 0.477 mmol/g resin by gravimetry.

165 mg of the resin loaded with Fmoc-Tyr-OH as above were allowed to swell for 2 hrs. in a 5 ml syringe with frit in NMP.

Fmoc-deprotection: With agitation, the resin is treated with 20% piperidine in NMP (3×10 min.) and then washed with NMP (5×2 min.) with agitation.

1^{st} Coupling

The Fmoc-protected sugar amino acid 1 (50,5 mg, 1.5 equiv) is dissolved in 2 ml of NMP together with HOAt (16 mg, 1.5 equiv), HATU (45 mg, 1.5 equiv) and collidine (156 μl, 15 equiv). This solution is charged into the syringe containing the Tyr-resin and allowed to react with agitation for 3-4 hours, followed by washing with NMP under agitation (5×1 min.) A few resin beads were taken from the syringe and treated with a few drops of a 20 vol.-% HFIP in DMC solution in an Eppendorf-Cap for 30 minutes. The dipeptide Fmoc-Z1-Tyr-OH thus separated from the resin was characterised through ESI mass spectrum: ESI-MS: 1237.6 [2M−H+Na+K]⁺; 1221.4 [2M−H+2Na]⁺; 1215.4 [2M+K]⁺; 1199.2 [2M+Na]⁺; 633.4 [M−H+2Na]⁺; 627.4 [M+K]⁺; 611.4 [M+Na]⁺; 589.3 [M+H]⁺.

2^{nd} Coupling

After Fmoc-deprotection and washing with NMP as described above, coupling was carried out for 2-3 hours with Fmoc-Thr(OTrt)-OH (115 mg, 2.5 equiv), HATU (75 mg, 2.5 equiv), HOAt (27 mg, 2.5 equiv) and 260 μl collidine in 2 ml of NMP with agitation, followed by washing with NMP (5×1 min) with agitation.

3^{rd} Coupling

After Fmoc-deprotection and washing with NMP as described above, coupling was carried out for 2-3 hrs. with Fmoc-Lys(ivDde)-OH (112.9 mg, 2.5 equiv), HATU (74.7 mg, 2.5 equiv), HOAt (27 mg, 2.5 equiv) and 260 μl of collidine in 2 ml of NMP with agitation, followed by washing with NMP (5×1 min) with agitation.

After washing with NMP, the resin is washed twice for DCM (1 min) and twice with MeOH (1 min.) and dried in vacuum over night. After that it is divided in equal parts and charged into 2 syringes (one for G and one for H) at 122 mg resin each. From this point onwards, synthesis of TG and TH is carried out separately.

4^{th} Coupling to Synthesise TG:

After Fmoc-deprotection and washing with NMP as described above, coupling was carried out with Fmoc-Trp-OH (50 mg, 3 equiv), HATU (45 mg, 3 equiv), HOAt (16 mg, 3 equiv) and 156 μl of collidine in 1 ml of NMP with agitation for 2-3 hrs., followed by washing with NMP (5×1 min.) with agitation.

4th Coupling to Synthesise TH:

After Fmoc-deprotection and washing with NMP as described above, coupling was carried out with Fmoc-D-Trp-OH (50 mg, 3 equiv), HATU (45 mg, 3 equiv), HOAt (16 mg, 3 equiv) and 156 μl of collidine in 1 ml of NMP with agitation for 2-3 hrs., followed by washing with NMP (5×1 min.) with agitation.

Cleavage of the Protected Linear Peptides Fmoc-Trp-Lys(ivDde)-Thr(OTrt)-Z1-Tyr-OH and Fmoc-D-Trp-Lys(ivDde)-Thr(OTrt)-Z1-Tyr-OH From the Resin:

After the Fmoc-deprotection and washing with NMP as described above, both peptides are washed with DCM (3×1 min.) with agitation and then separated from the resin with 20 vol-% HFIP in DCM (3×20 min.) with agitation.

The DCM is removed under reduced pressure. In each case characterisation is carried out through HPLC-MS:

H-Trp-Lys(ivDde)-Thr(OTrt)-Z1-Tyr-OH: ESI-MS: 1306.4 [M−H+2K]⁺; 1290.5 [M−H+Na+K]⁺; 1274.6 [M−H+2Na]⁺; 1268.6 [M+K]⁺; 1252.6 [M+Na]⁺; 1230.4 [M+H]⁺; 988.5 [M-Trt+H]⁺; 930.5 [M−Trt-acetone+H]⁺; 243 [Trt]⁺; t_{R}=12.90 min (HPLC-MS, 40-90%B in 15 min).

H-D-Trp-Lys(ivDde)-Thr(OTrt)-Z1-Tyr-OH: ESI-MS: 1306.4 [M−H+2K]⁺; 1290.5 [M−H+Na+K]⁺; 1274.6 [M−H+2Na]⁺; 1268.6 [M+K]⁺; 1252.6 [M+Na]⁺; 1230.4 [M+H]⁺; 988.5 [M−Trt+H]⁺; 930.5 [M−Trt-acetone+H]⁺; 243 [Trt]⁺; t_{R}=12.97 min (HPLC-MS, 40-90%B in 15 min).

Cyclisation

The peptides H-Trp-Lys(ivDde)-Thr(OTrt)-Z1-Tyr-OH and H-D-Trp-Lys(ivDde)-Thr(OTrt)-Z1-Tyr-OH were dissolved in 12 ml of DMF each. 37.9 μl of DPPA and 25 mg of NaHCO₃ were added with stirring. After 12 hrs., the reaction was completed.

c[-Trp-Lys(ivDde)-Thr(OTrt)-Z1-Tyr-]: ESI-MS: 1256.7 [M−H+2Na]⁺; 1250.7 [M+K]⁺; 1234.7 [M+Na]⁺; 1219.0 [M+Li]⁺; 970.5 [M−Trt+H]⁺; 912.6 [M−Trt-acetone+H]⁺; 243 [Trt]⁺; t_{R}=22.13 min (HPLC-MS, 30-70%B in 15 min).

c[-D-Trp-Lys(ivDde)-Thr(OTrt)-Z1-Tyr-]: ESI-MS: 1256.7 [M−H+2Na]⁺; 1250.7 [M+K]⁺; 1234.8 [M+Na]⁺; 1218.8 [M+Li]⁺; 970.6 [M−Trt+H]⁺; 912.7 [M−Trt-acetone+H]⁺; 243 [Trt]⁺; t_{R}=22.28 min (HPLC-MS, 30-70%B in 15 min).

ivDde-Deprotection of the Lysine Side Chain:

The protected cyclopeptides are dissolved 3×in 3% hydrazine in DMF solution, reacted with stirring for 10 min. and the solvent removed under reduced pressure. The residue was solubilised with a few drops of DMF and these and the peptide precipitated with diethyl ether. Purification in each case was carried out by semi-preparative HPLC. After lyophilisation both peptides were present as an amorphous white powder.

c[-Trp-Lys-Thr(OTrt)-Z1-Tyr-] (TG): Semi-preparative HPLC purification: Gradient: 40-65%B in 30 min; (B=90% acetonitrile, 10% water, +0.1% TFA) ESI-MS: 1044.5 [M+K]⁺; 1028.5 [M+Na]⁺; 1006.2 [M+H]⁺; 764.4 [M−Trt+H]⁺; 706.4 [M−Trt-acetone+H]⁺; 243.2 [Trt]⁺; t_{R}=13.94 min (HPLC-MS, 30-70%B in 15 min; B=acetonnitrile+0.1% TFA).

c[-D-Trp-Lys-Thr(OTrt)-Z1-Tyr-] (TH): Semi-preparative HPLC purification: Gradient: 50-65%B in 30 min; (B=90% acetonitrile, 10% water, +0.1% TFA) ESI-MS: 1044.5 [M+K]⁺; 1028.6 [M+Na]⁺; 1012.6 [M+Li]⁺; 764.4 [M−Trt+H]⁺; 706.4 [M−Trt-acetone+H]⁺; 243 [Trt]⁺; t_{R}=14.35 min (HPLC-MS, 30-70%B in 15 min; B=acetonitrile+0.1% TFA).

### Example 3

### SGnc 18: c[-D-Trp-Lys-Thr(OTrt)-Z2-Phe-]

Loading with Resin:

TCP-resin (2 g) was loaded with 933 mg (1.2 equiv) of Fmoc-Phe-OH, DIPEA (2.5 equiv, 1.05 ml) in 16 ml of DCM in a 20 ml syringe according to standard methods. By gravimetry, the loading was determined to be 0.677 mmol/g resin. 52.4 mg of the Fmoc-Phe-OH loaded resin were allowed to swell with frit in a 2 ml syringe in NMP for two hrs.

Fmoc-deprotection: With agitation the resin is treated with 20% piperidine in NMP (3×10 min.) and then washed with NMP (5×2 min.) with agitation.

1^{st} Coupling

The Fmoc-protected sugar amino acid 2 (24.3 mg, 1.5 equiv) is dissolved in 194 μl of DMF together with HOAt (7.3 mg, 1.5 equiv), HATU (20.25 mg, 1.5 equiv) and collidine (70.7 μl, 15 equiv). This solution is charged into the syringe containing the Phe-resin and allowed to react with agitation for 3-4 hours, followed by washing with NMP under agitation (5×1 min.) A few resin beads were taken from the syringe and treated with a few drops of a 20 vol.-% HFIP in DCM solution in an Eppendorf-Cap for 30 minutes. The dipeptide Fmoc-Z1-Tyr-OH thus separated from the resin was characterised through an ESI mass spectrum: ESI-MS: 1249.3 [2M−H+2Na]⁺; 1227.2 [2M+Na]⁺; 1204.9 [2M+H]⁺; 663.4 [M−H+Na+K]⁺; 647.4 [M−H+2Na]⁺; 641.3 [M+K]⁺; 625.4 [M+Na]⁺; 603.2 [M+H]⁺.

2ⁿ Coupling

After Fmoc-deprotection and washing with NMP as described above, coupling was carried out for 2-3 hours with Fmoc-Thr(OTrt)-OH (42 mg, 2 equiv), HATU (27 mg, 2 equiv), HOAt (9.5 mg, 2.5 equiv) and 95 pi of collidine (20 equiv) in NMP (250 μl) with agitation, followed by washing with NMP (5×1 min) with agitation.

3^{rd} Coupling

After Fmoc-deprotection and washing with NMP as described above, coupling was carried out for 2-3 hrs. with Fmoc-Lys(ivDde)-OH (41 mg, 2 equiv), HATU (27 mg, 2 equiv), HOAt (9.5 mg, 2 equiv) and 95 μl of collidine (20 equiv) in 250 μl of NMP with agitation, followed by washing with NMP (5×1 min) with agitation.

4^{th} Coupling

After Fmoc-deprotection and washing with NMP as described above, coupling was carried out with Fmoc-Trp-OH (30.2 mg, 2 equiv), HATU (25,7 mg, 2 equiv), HOAt (9.7 mg, 2 equiv) and 94 μl of collidine in NMP with agitation for 2-3 hrs., followed by washing with NMP (5×1 min.) with agitation.

Cleavage of the Protected Linear Peptide Fmoc-D-Trp-Lys(ivDde)-Thr(OTrt)-Z2-Phe-OH From the Resin

After Fmoc-deprotection and washing with NMP as described above, the peptide was washed with DCM with agitation (3×1 min.) and then separated from the resin with 20 vol-% of HFIP in DCM (3×20 min) with agitation. The DCM is removed under reduced pressure.

Cyclisation

The peptide H-D-Trp-Lys(ivDde)-Thr(OTrt)-Z2-Phe-OH was dissolved in 7.1 ml of DMF and 23 Al of DPPA and 4.9 mg NaHCO₃ added with agitation. After 12 hrs., the reaction was completed (no linear peptide visible in the ESI mass spectrum).

ivDde-Deprotection of the Lysine Side Chain

The protected cyclopeptide was dissolved 3×in 3% hydrazine in DMF solution, reacted with stirring for 10 min. and the solvent removed under reduced pressure. The residue was solubilised with a few drops of DMF and added dropwise to diethyl ether to precipitate the peptide. Purification in each case was carried out by semi-preparative HPLC. After lyophilisation the peptide was present as an amorphous white powder.

c[-D-Trp-Lys-Thr(OTrt)-Z2-Phe-] (SGnc 18): Semi-preparative HPLC purification: Gradient: 50-65%B in 30 min; (B=90% acetonitrile, 10% water, +0,1% TFA)

### Example 4

Parallel synthesis of SGnc 12: c[-D-Trp-Lys-Phe(F₅)-Z1-Phe-]; SGnc 13: c[-D-Trp-Lys-Bip-Z1-Phe-]; SGnc 14: c[-D-Trp-Lys-Bpa-Z1-Phe-]; SGnc 15: c[-D-Trp-Lys-1-Nal-Z1-Phe-]; SGnc 16: c[-D-Trp-Lys-2-Nal-Z1-Phe-]:

Loading With Resin:

TCP-resin (2 g) was loaded with 933 mg (1.2 equiv) of Fmoc-Phe-OH, DIPEA (2.5 equiv, 1.05 ml) in 16 ml of DCM in a 20 ml syringe according to standard methods. By gravimetry, the loading was determined to be 0.677 mmol/g resin.

52.4 mg of the Fmoc-Phe-OH loaded resin each were weighed and charged into a 2 ml syringe and allowed to swell in NMP for two hrs.

Fmoc-deprotection: With agitation the resin in each of the 5 syringes is treated with 20% piperidine in NMP (3×10 min.) and then washed with NMP (5×2 min.) with agitation.

1st Coupling

The Fmoc-protected sugar amino acid 1 (113.5 mg, 1.5 equiv) is dissolved in 1 ml of DMF together with HOAt (36.3 mg, 1.5 equiv), HATU (101.3 mg, 1.5 equiv) and collidine (353 μl, 15 equiv). This solution is charged in equal parts, i.e. 270.7 ill each, into 5 syringes containing the Phe-resin and allowed to react with agitation for 3-4 hours, followed by washing with NMP under agitation (5×1 min.) By way of an example, a few resin beads were taken from the syringe to synthetise SGnc 13 and treated with a few drops of a 20 vol.-% HFIP in DCM solution in an Eppendorf-Cap for 30 minutes. The dipeptide Fmoc-Z1-Phe-OH thus separated from the resin was characterised through an ESI mass spectrum:

ESI-MS: 1738.7 [3M+Na]⁺; 1716.8 [3M+H]⁺; 1205.4 [2M−H+Na+K]⁺; 1167.1 [2M+Na]⁺; 1144.9 [2M+H]⁺; 611.3 [M+K]⁺; 595.3 [M+Na]⁺; 573.2 [M+H]⁺.

2^{nd} Coupling:

After Fmoc-deprotection and washing with NMP as described above, coupling with agitation was carried out for 2-3 hrs. each

to synthesise SGnc 12: with 33.9 mg of Fmoc-Phe(F₅)-OH, 27 mg of HATU, 10 mg of HOAt and 94 μl of collidine in 300 μl NMP;

to synthesise SGnc 13: with 33.0 mg of Fmoc-Bip-OH, 27 mg of HATU, 10 mg of HOAt and 94 μl of collidine in 300 μl NMP;

to synthesise SGnc 14: with 35 mg of Fmoc-Bpa-OH, 27 mg of HATU, 10 mg of HOAt and 94 μl of collidine in 300 μl of NMP;

to synthesise SGnc 15: with 31 mg of Fmoc-1-Nal-OH, 27 mg of HATU, 10 mg of HOAt and 94 μl of collidine in 300 μl of NMP;

to synthesise SGnc 16: with 31 mg of Fmoc-2-Nal-OH, 27 mg of HATU, 10 mg o HOAt and 94 μl collidine in 300 μl of NMP;

After that, washing with NMP (5×1 min.) was carried with agitation.

3^{rd} Coupling:

After Fmoc-deprotection and washing with NMP as described above, coupling was carried out for 2 to 3 hrs. as follows:

to synthesise SGnc 12-14 and SGnc 16: Fmoc-Lys(ivDde)-OH (163 mg, 2 equiv), HATU (108 mg, 2 equiv), HOAt (38 mg, 2 equiv) and 377 μl of collidine (20 equiv) are dissolved in 1.3 ml of NMP. This solution is charged into the pertinent syringe in equal parts, i.e. 419 μl each, and subjected to coupling with agitation.

to synthesise SGnc 15: Fmoc-Lys(ivDde)-OH (40.75 mg, 2 equiv), HATU (27 mg, 2 equiv), HOAt (9 mg, 2 equiv) and 94 μl of collidine (20 equiv) is dissolved in 300 μl of NMP gelöst, charged into the syringe and subjected to coupling with agitation.

After that, washing with NMP was carried out with agitation (5×1 min.).

4^{th} Coupling:

After Fmoc-deprotection and washing with NMP as described above, coupling was carried out for 2 to 3 hrs. as follows:

to synthesise SGnc 12-14 und Sgnc 16: Fmoc-D-Trp-OH (121 mg, 2 equiv), HATU (108 mg, 2 equiv), HOAt (38 mg, 2 equiv) and 377 μl of collidine (20 equiv) are dissolved in 1.3 ml NMP. This solution is drawn into the pertinent syringe in equal parts, i.e. 419 μl and subjected to coupling with agitation.

to synthesise SGnc 15: Fmoc-D-Trp-OH (30.2 mg, 2 equiv), HATU (27 mg, 2 equiv), HOAt (9 mg, 2 equiv) and 94 μl of collidine (20 equiv) are dissolved in 300 μl of NMP, drawn into the syringe and subjected to coupling with agitation.

After that, washing with NMP was carried out with agitation (5×1 min.)

Cleavage of the Protected Linear Peptides from the Resin

After Fmoc-deprotection and washing with NMP as described above, the peptides were washed DCM with agitation (3×1 min.) and then separated from the resin with 20 vol-% each of HFIP in DCM (3×20 min) with agitation. The DCM is removed under reduced pressure.

Cyclisation

The protected linear peptides were dissolved in 7.1 ml of DMF each and 23 μl of DPPA and 4.9 mg of NaHCO₃ each added with agitation. After 12 hrs., the reaction was completed (no linear peptide visible in the ESI mass spectrum). Exemplary characterisation of the ivDde-protected SGnc 12: c[-D-Trp-Lys(ivDde)-Phe(F₅)-Z1-Phe-] by ESI-MS:

1134.6 [M−H+2Na]⁺; 1128.6 [M+K]⁺; 1112.7 [M+Na]⁺; 1090.6 [M+H]⁺; 1032.6 [M-acetone+H]⁺.

ivDde-Deprotection of the Lysine Side Chain

The cyclopeptides ivDde-protected in the lysine side chain were dissolved 3×in 3% hydrazine in DMF solution, reacted with stirring for 10 min. and the solvent removed under reduced pressure. The residue was solubilised with a few drops of DMF each and the peptide precipitated with diethyl ether. Purification in each case was carried out by semi-preparative HPLC. After lyophilisation all of the peptides were present as an amorphous white powder.

c[-D-Trp-Lys-Phe(F₅)-Z1-Phe-] (SGnc 12): Semi-preparative HPLC purification: Gradient: 30-70%B in 30 min; (13=90% acetonitrile, 10% water, +0.1% TFA) t_{R}=24.35

ESI-MS: 1806.4 [2M(1*¹³C)+K]⁺; 1805.4 [2M+K]⁺; 1790.3 [2M(1*¹³C)+Na]⁺; 1789.3 [2M+Na]⁺; 1768.2 [2M(1*¹³C)+H]⁺; 1767.2 [2M+H]⁺; 922.3 [M+K]⁺; 906.4[M+Na]⁺; 884.3 [M+H]⁺; 826.4 [M-acetone+H]⁺; t_{R}=11.65 min (HPLC-MS, 30-70%B in 15 min; B=acetonitrile+0.1% TFA).

c[-D-Trp-Lys-Bip-Z1-Phe-] (SGnc 13): Semi-preparative HPLC purification: Gradient: 45-63%B in 30 min; (B=90% acetonitrile, 10% water, +0.1% TFA)

ESI-MS: 1028.2 [M+TFA−H+2Na]⁺; 1022.4 [M+TFA+K]⁺; 1006.5 [M+TFA+Na]⁺; 908.4 [M+K]⁺; 892.6 [M+Na]⁺; 870.4 [M+H]⁺; 812.5 [M-acetone+H]⁺; t_{R}=13.05 min (HPLC-MS, 30-70%B in 15 min; B=acetonitrile+0.1% TFA).

c[-D-Trp-Lys-Bpa-Z1-Phe-] (SGnc 14): Semi-preparative HPLC purification: Gradient: 45-65%B in 30 min; (B=90% acetonitrile, 10% water+0.1% TFA); t_{R}=17.5 min;

ESI-MS: 1056.1 [M+TFA−H+2Na]⁺; 1050.3 [M+TFA+K]⁺; 1034.4 [M+TFA+Na]⁺; 936.5 [M+K]⁺; 920.6 [M+Na]⁺; 898.4 [M+H]⁺; 840.5 [M-acetone+H]⁺.

SGnc 15: ESI-MS: 1840.7 [2M(1*¹³C)+TFA+K]⁺; 1710.6 [2M(1*¹³C)+Na]⁺; 1687.5 [2M+H]⁺; 1002.1 [N+TFA−H+2Na]⁺; 996.4 [M+TFA+K]⁺; 980.3 [M+TFA+Na]⁺; 882.5 [M+K]⁺; 866.6 [M+Na]⁺; 844.4 [M+H]⁺; 786.5 [M-acetone+H]⁺. t_{R}=3.75 min (HPLC-MS, 30-70%B in 15 min; B=MeCN +0.1%TFA).

c[-D-Trp-Lys-2-Nal-Z1-Phe-] (SGnc 16): Semi-preparative HPLC purification: Gradient: 45-65%B in 30 min; (B=90% acetonitrile, 10% water, +0.1% TFA)

ESI-MS: 1839.6 [2M+TFA+K]⁺; 1709.6 [2M+Na]⁺; 1687.6 [2M+H]⁺; 1002.1 [M+TFA−H+2Na]⁺; 996.4 [M+TFA+K]⁺; 980.4 [M+TFA+Na]⁺; 882.5 [M+K]⁺; 866.6 [M+Na]⁺; 844.4 [M+H]⁺; 786.5 [M-acetone+H]⁺.

### Example 5

General procedure for anchoring of the first Fmoc-protected amino acid on TCP resin (GP 2): The unloaded dry TCP resin in a syringe (exact weight known), completed with a frit, was swelled in NMP (30 min). The resin was filtered off, before a solution (˜0.125 M) of 1.2 equiv of Fmoc-protected amino acid (with respect of the theoretical capacity of the TCP resin) and 2.5 equiv DIPEA (with respect to the quantity of Fmoc-protected amino acid used) in DCM (abs.) was added. After shaking for 1 h at rt the capping solution (20% DIPEA in MeOH) is added. After 15 min the resin is filtered off, and the resin is washed with DCM (3×3 min), DMF (3×3 min), and MeOH (3×3 min), and dried overnight under vacuo. Subsequently the exact weight of the dried resin was determined, and the loading of the resin was calculated:

c[mol/g]=(mₜₒₜₐₗ−mᵣₑₛᵢₙ)/{MG_{Xaa}−36.461)×mₜₒₜₐₗ

c loading

mᵣₑₛᵢₙ mass of resin before loading

mₜₒₜₐₗ mass of loaded resin

MG_{Xaa} molar weight of the Fmoc-protected amino acid (Xaa)

General Procedure for Solid-Phase Peptide Synthesis (GP 3)

The preloaded resin was swelled for 30 min in NMP. The Fmoc-protecting group of the amino acid attached to the resin is removed by treating the resin with a 20% piperidine solution in DMF (3×10 min). The resin is filtered off and washed with NMP (5×3 min), before a solution of the next Fmoc-protected amino acid (3 equiv), or Fmoc-Z-OH (that is in the following examples either Fmoc-Z1-OH or Fmoc-Z2-OH) (1.5 equiv), HATU and HOAt (L. A. Carpino, A. El-Faham, F. Albericio, Tetrahedron Lett. 1994, 35, 2279-2282; L. A. Carpino, A. El-Faham, C. A. Minor, F. Albericio, J. Chem. Soc. Chem. Commun. 1994, 2, 201-203) (1.5 equiv each for SAA coupling, 3 equiv each for other amino acids), and 2,4,6-collidine (15 equiv/30 equiv) in NMP (for coupling with Fmoc-protected Z1 DMF, was used as solvent) is added. After 2-3 h reaction is complete (monitoring by ESI-HPLC-MS). The resin is washed with NMP (5×3 min), prior to the subsequent Fmoc-deprotection and coupling steps. After coupling of the last amino acid, and subsequent Fmoc-deprotection, the resin is washed with NMP (3×3 min), CH₂Cl₂ (1×3 min), and dried overnight in vacuo. The compounds are cleaved from the dry resin using 20% HFIP solution in CH₂Cl₂ (3×10 min)(R. Bollhagen, M. Schmiedberger, K. Barlos, E. Grell, J. Chem. Soc., Chem. Commun. 1994, 22, 2559-2560). The crude peptides were purified via RP-HPLC. In all cases peptide (HPLC) purity was >99%.

General procedure for cyclization with DPPA/NaHCO₃ (GP 4):

The Fmoc-deprotected linear peptide is dissolved in DMF (0.1 mM), and DPPA (3 equiv) and NaHCO₃ (11 equiv) are added (T. Shioiri, K. Ninomiya, S. Yamada, J. Am. Chem. Soc. 1972, 94, 6203-6205; S. F. Brady, W. J. Paleveda, B. H. Arison, R. M. Freidinger, R. F. Nutt, D. F. Veber, in 8th Am. Pept. Symp. (Eds.: V. J. Hruby, D. H. Rich), Pierce Chem. Co., Rockford, Ill., USA, Tuscon, Ariz., USA, 1983, pp. 127-130). After 12 h reaction is usually complete. After side chain deprotection (c. f. GP 5) the cyclic peptides were precipitated with Et₂O and purified via RP-HPLC, and finally lyophilized from water or dioxane.

General Procedure for ivDde Deprotection (GP 5):

The peptide is dissolved in 3% hydrazine/DMF solution, stirred for 10-15 min, and the solvent is evaporated. This procedure is repeated 3 times.

Synthesis of the First Library of Somatostatin Analogues SGA, SGB, SGE, SGF

Loading of the TCP Resin With Fmoc-Phe-OH:

For the syntheses of

cyclo[-Phe-Trp-Lys-Z1-] SGA,

### cyclo[-Phe-DTrp-Lys-Z1-] SGB,

According to GP 2, TCP resin (2.008 g) was loaded with Fmoc-Phe-OH (933.6 mg, 2.4098 mmol) and DIPEA (1.05 mL, 6.025 mmol) in 16 mL DCM. The loading was c=0.677 mol/g resin.

Loading of the TCP Resin With Fmoc-Tyr-OH:

For the syntheses of

cyclo[-Tyr-Trp-Lys-Z1-] SGE,

cyclo[-Tyr-DTrp-Lys-Z1-]SGF,

Similar to GP 2 (instead of DIPEA 2,4,6-collidine was used as base), TCP resin (1.300 g) was loaded with Fmoc-Tyr-OH (629 mg, 1.56 mmol) and 2,4,6-collidine (2.77 mL) in 10 mL DCM. The loading was c=0.477 mmol/g resin.

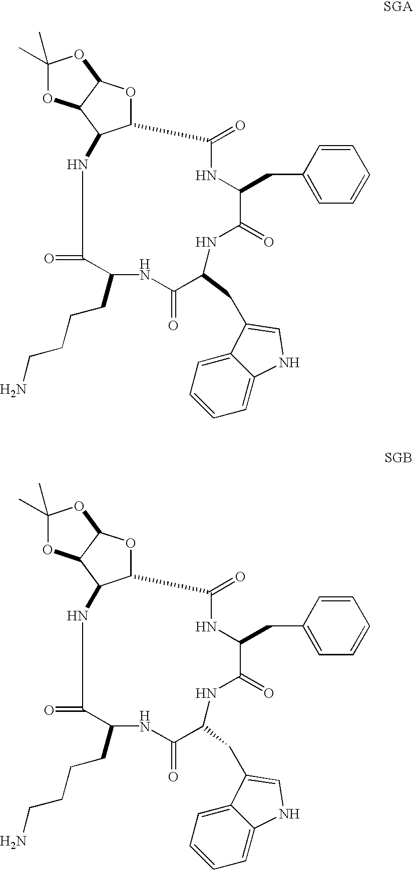

Synthesis of SGA and SGB: According to GP 3, SGA and SGB were synthesized parallel in the same syringe ((2 mL), 137 mg of the Fmoc-Phe-OH loaded TCP resin). Coupling was verified by a sample cleavage of the dipeptide Fmoc-Z1-Phe-OH: ESI-MS: 1205.6 [2M-H+Na+K]⁺; 1167.2 [2M+Na]⁺; 1144.9 [2M+H]⁺; 611.4 [M+K]⁺; 595.4 [M+Na]⁺; 573.3 [M+H]⁺; t_{R}=25.04 min (anal. HPLC, 20-80%B in 30 min). The first coupling was done with Fmoc-protected Z1 (60.8 mg), HOAt (18.9 mg), HATU (53 mg) and 2,4,6-collidine (184 μL). Subsequentely Fmoc-Lys(ivDde)-OH (133 mg) (HOAt (31.6 mg), HATU (88.2 mg), 2,4,6-collidine (307 μL)) was coupled. The resin was split into two equal parts—one for the synthesis of SGA, one for the synthesis of SGB. Coupling with Fmoc-L-Trp-OH, or Fmoc-D-Trp-OH (59.4 mg of L-, or D-Trp respectively) (HOAt (18.9 mg), HATU (52.9 mg), 2,4,6-collidine (184 μL))respectively, and subsequent washing Fmoc-deprotection and cleavage steps (GP 3) yielded the linear, ivDde-protected precursors of compounds SGA and SGB, characterized by HPLC-MS:

H₂N-Trp-Lys(ivDde)-Z1-Phe-OH (precursor to SGA): 909.5 [M+K]⁺; 893.5 [M+Na]⁺; 871.5 [M+H]⁺. 813.5; [M-acetone +H]⁺; t_{R}=11.41 min (HPLC-MS, 30-90%B in 15 min), t_{R}=14.41 min (anal. HPLC, 30-90%1B in 15 min).

H₂N-DTrp-Lys(ivDde)-Z1-Phe-OH (precursor to SGB): 915.5 [M−H+2Na]⁺; 909.5 [M+K]⁺; 893.5 [M+Na]⁺; 871.5 [M+H]⁺. 813.5; [M-acetone+H]⁺; t_{R}=11.31 min (HPLC-MS, 30-90%B in 15 min).

The precursors to SGA and SGB were cyclizied according to GP 4 (DPPA (37.9 μL), NaHCO₃ (25 mg), DMF (12 mL)) to yield the protected cyclic precursors:

cyclo[-Trp-Lys(ivDde)-Z1-Phe-] (precursor of SGA): ESI-MS: 1729.0 [2M+Na]⁺; 890.6 [M+K]⁺; 875.7 [M+Na]⁺; 853.6 [M+H]⁺; 795.6 [M-acetone+H]⁺; t_{R}=19.19 min (anal. HPLC, 30-90%B).

cyclo[-D-Trp-Lys(ivDde)-Z1-Phe-] (precursor of SGB): ESI-MS: 1743.1 [2M+K]⁺; 1729.0 [2M+Na]⁺; 1705.6 [2M+H]⁺; 897.6 [M−H+2Na]⁺; 891.7 [M+K]⁺; 875.7 [M+Na]⁺; 853.6 [M+H]⁺; 795.6 [M-acetone+H]⁺; t_{R}=21.32 min (anal. HPLC, 10-60%B).

ivDde-deprotection according to GP 5, purification via rp-HPLC (semipreparative; gradient: 35-55% B in 30 min (SGA), and 20-60%B in 30 min (SGB), respectively; (B=90% MeCN, 10% H₂O, +0.1%TFA)), and subsequently lyophilization yielded the compounds SGA (10 mg, 33%) and SGB (10.7 mg, 36%) as white, fluffy powder.

SGA: ESI-MS: 1445.1 [2M+TFA+K]⁺; 1331.3 [2M+K]⁺; 1315.2 [2M+Na]⁺; 1293.2 [2M+H]⁺; 799.1 [M+TFA+K]⁺; 783.1 [+TFA+Na]⁺; 685.2 [M+K]⁺; 669.4 [M+Na]⁺; 647.2 [M+H]⁺; 589.2 [M-acetone+H]⁺; t_{R}=4.78 min (HPLC-MS, 30-70%B in 15 min; B=MeCN+0.1%TFA).

SGB: ESI-MS: 1445.2 [2M+TFA+K]⁺; 1331.4 [2M+K]⁺; 1315.3 [2M+Na]⁺; 1293.3 [2M+H]⁺; 799.1 [M+TFA+K]⁺; 669.3 [M+Na]⁺; 647.2 [M+H]⁺; 589.3 [M-acetone+H]⁺; t_{R}=5.74 min (HPLC-MS, 30-70%B in 15 min; B=MeCN+0. 1%TFA).

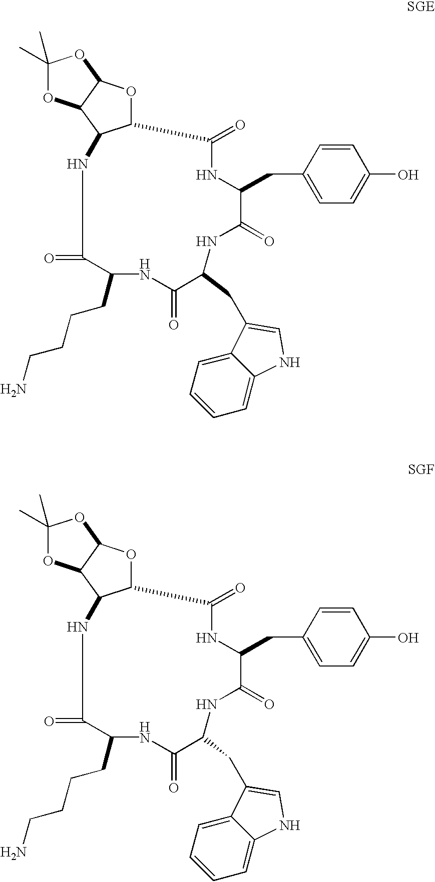

Synthesis of SGE and SGF: According to GP 3, SGE and SGF were synthesized parallel in the same syringe (2 mL), 190 mg of the Fmoc-Tyr-OH loaded TCP resin). The first coupling was done with Fmoc-protected Z1 (58 mg), HOAt (18.5 mg), HATU (52 mg) and 2,4,6-collidine (180 μL). Coupling was verified by a sample cleavage: Some beads were fished out, and the dipeptide Fmoc-Z1-Tyr-OH cleaved from those beads in an Eppendorf cap according to GP 3. Characterization: ESI-MS: 1237.6 [2M−H+Na+K]⁺; 1221.4 [2M−H+2Na]⁺; 1215.4 [2M+K]⁺; 1199.2 [2M+Na]⁺; 921.6 [(3M+2K)/2]²⁺; 913.7 [(3M+Na⁺ K)/2]²⁺; 633.4 [M−H+2Na]⁺; 627.4 [M+K]⁺; 611.4 [M+Na]⁺; 589.3 [M+H]⁺; t_{R}=21.68 min (anal. HPLC, 20-80%B in 30 min). According to GP 3 Fmoc-Lys(ivDde)-OH (130 mg) (HOAt (31 mg), HATU (86 mg), 2,4,6-collidine (300 μL)) was coupled. The resin was split into two equal parts—one for the synthesis of SGE, one for the synthesis of SGF. Coupling with Fmoc-L-Trp-OH, or Fmoc-D-Trp-OH (58 mg of L-, or D-Trp respectively) (HOAt (18.5 mg), HATU (52 mg), 2,4,6-collidine (180 μL))respectively, and subsequent washing Fmoc-deprotection and cleavage steps (GP 3) yielded the linear, ivDde-protected precursors of compounds SGE and SGF. The precursors to SGE and SGF were cyclizied according to GP 4 (DPPA (38 μL), NaHCO₃ (25 mg), DMF (12 mL)) to yield the protected cyclic precursors:

cyclo[-Trp-Lys(ivDde)-Z1-Tyr-] (precursor of SGE): ESI-MS: 1759.9 [2M+Na]⁺; 906.7 [M+K]⁺; 891.6 [M+Na]⁺; 869.6 [M+H]⁺; 811.6 [M-acetone+H]⁺; t_{R}=11.89 min (anal. HPLC, 30-90%B, 30 min).

cyclo[-D-Trp-Lys(ivDde)-Z1-Tyr-] (precursor of SGF): ESI-MS: 906.7 [M+K]⁺; 891.6 [M+Na]⁺; 869.6 [M+H]⁺; 811.6 [M-acetone+H]⁺; t_{R}=11.74 min (anal. HPLC, 30-90%B, 30 min).

ivDde-deprotection according to GP 5, purification via rp-HPLC (semipreparative; gradient: 20-60% B in 30 min (SGE), and 25-60%B in 30 min (SGF), respectively; (B=90% MeCN, 10% H2O, +0.1%TFA)), and subsequently lyophilization yielded the compounds SGE and SGF as white, fluffy powder.

SGE: ESI-MS: 799.2 [M+TFA+Na]⁺; 685.4 [M+Na]⁺; 663.2 [M+H]⁺; 605.3 [M-acetone+H]⁺; t_{R}=15.46 min (anal. HPLC, 20-60%B in 15 min; B=MeCN+0.1%TFA).

SGF: ESI-MS: 1363.3 [2M+K]⁺; 1347.1 [2M+Na]⁺; 1325.2 [2M+H]⁺; 685.4 [M+Na]⁺; 663.3 [M+H]⁺; 605.3 [M-acetone+H]⁺; t_{R}=20.19 min (anal. HPLC, 10-60%B in 15 min; B=MeCN+0.1%TFA).

Synthesis of SGnc 7: cyclo[-D-Trp-Nle-Thr(OTrt)-Z1-Tyr-]

SGnc 7 was synthesized according to GP 3 (2 mL, 66.8 mg of the Fmoc-Tyr-OH loaded TCP resin). Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. Coupling of the Fmoc-Thr(OTrt)-OH, was verified by a sample cleavage: Some beads were fished out, and the tripeptide Fmoc-Thr(OTrt)-Z1-Tyr-OH cleaved from those beads in an Eppendorf cap according to GP 3. ESI-MS of that sample cleavage: 1885.3 [²M+Na]⁺; 1863.0 [2M+H]⁺; 976.4 [M−H+2Na]⁺; 970.4 [M+K]⁺; 954.4 [M+Na]⁺; 932.4 [M+H]⁺; 243.2 [Trt]+. According to GP 3 Fmoc-Nle-OH, and Fmoc-D-Trp-OH were coupled consecutively. Subsequent cleavage from the resin (GP 3), cyclization according to GP 4, and purification via RP-HPLC (semipreparative; gradient: 50-100%B in 30 min), yielded the SGnc 7 as a white fluffy powder: ESI-MS: 1998.7[2M+Li]⁺; 1143.4 [M−H+TFA+K]⁺; 1127.5 [M−H+TFA+Na]⁺; 1029.5 [M+K]⁺; 1013.5 [M+Na]⁺; 997.7 [M+Li]⁺; 990.6 [M+H]⁺; 771.7 [M−Trt+Na]⁺; 749.4 [M−Trt+H]⁺; 691.4 [M−Trt-acetone+H]⁺; 243.2 [Trt]⁺. t_{R}=21.05 min (HPLC-MS, 30-70%B in 15 min).

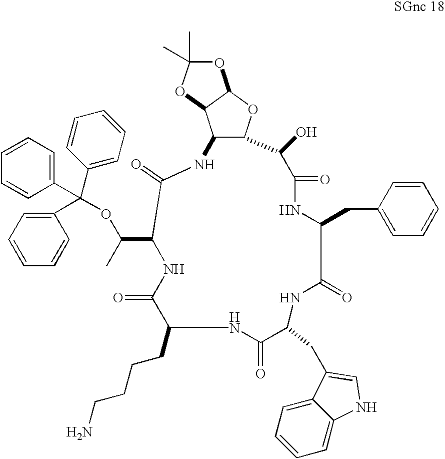

Synthesis of SGnc 18: cyclo[-D-Trp-Lys-Thr(OTrt)-Z2-Phe-]

SGnc 18 was synthesized according to GP 3 (2 mL, 52.4 mg of the Fmoc-Phe-OH loaded TCP resin). Coupling of the Fmoc-protected Z2 was verified by a sample cleavage: Some beads were fished out, and the dipeptide Fmoc-Z2-Phe-OH cleaved from those beads in an Eppendorf cap according to GP 3. ESI-MS of that sample cleavage: 1829.8 [3M+Na]⁺; 1227.2 [2M+Na]⁺; 1205.0 [2M+H]⁺; 663.4 [M−H+Na+K]⁺; 647.4 [M−H+2Na]⁺; 641.3 [M+K]⁺; 625.4 [M+Na]⁺; 603.2 [M+H]⁺; 551.3 [M-acetone+Li]⁺; 545.1 [M-acetone+H]⁺. According to GP 3 Fmoc-Thr(OTrt)-OH, Fmoc-Lys(ivDde)-OH, and Fmoc-D-Trp-OH were coupled consecutively. Subsequent cleavage from the resin (GP 3), cyclization according to GP 4, the ivDde cyclic precursor cyclo[-D-Trp-Lys(ivDde)-Thr(OTrt)-Z2-Phe-]: ESI-MS: 1264.8 [M+K]⁺; 1248.9 [M+Na]⁺; 1226.5 [M+H]⁺; 1006.8 [M−Trt+Na]⁺; 984.6 [M−Trt+H]⁺; 926.7 [M−Trt-acetone+H]⁺; 243.2 [Trt]⁺. Subsequent ivDde deprotection according to GP 5, and purification via RP-HPLC (semipreparative; gradient: 50-65%B in 30 min), yielded the SGnc 18 as a white fluffy powder: ESI-MS: 1058.3 [M+K]⁺; 1042.5 [M+Na]⁺; 1020.2 [M+H]⁺; 800.6 [M−Trt+Na]⁺; 778.4 [M−Trt+H]⁺; 720.4 [M−Trt-acetone+H]⁺; 243.2 [Trt]⁺. t_{R}=15.18 min (HPLC-MS, 30-70%B in 15 min).

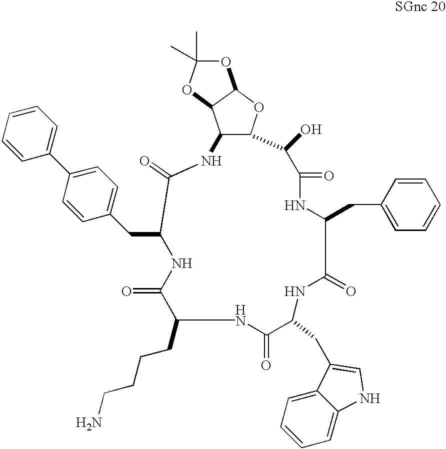

Synthesis of SGnc 20: cyclo[-D-Trp-Lys-Thr(OTrt)-Z2-Phe-]

SGnc 20 was synthesized according to GP 3 (2 mL, 52.4 mg of the Fmoc-Phe-OH loaded TCP resin). Coupling of the Fmoc-protected Z2 was verified by a sample cleavage: Some beads were fished out, and the dipeptide Fmoc-Z2-Phe-OH cleaved from those beads in an Eppendorf cap according to GP 3. ESI-MS of that sample cleavage: 1829.8 [3M+Na]⁺; 1227.2 [2M+Na]⁺; 1205.0 [2M+H]⁺; 663.4 [M−H+Na+K]⁺; 647.4 [M−H+2Na]⁺; 641.3 [M+K]⁺; 625.4 [M+Na]⁺; 603.2 [M+H]⁺; 551.3 [M-acetone+Li]⁺; 545.1 [M-acetone+H]⁺. According to GP 3 Fmoc-Bip-OH, Fmoc-Lys(ivDde)-OH, and Fmoc-D-Trp-OH were coupled consecutively. Subsequent cleavage from the resin (GP 3), cyclization according to GP 4, ivDde-deprotection and purification via RP-HPLC (semipreparative; gradient: 50-65%B in 30 min), yielded SGnc 20 as a white fluffy powder: ESI-MS: 938.9 [M+K]⁺; 922.9 [M+Na]⁺; 900.7 [M+H]⁺; 842.7 [M-acetone+H]⁺. t_{R}=13.10 min (HPLC-MS, 30-70%B in 15 min).

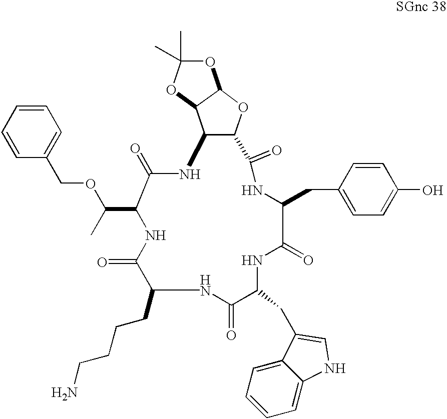

Synthesis of SGnc 38: cyclo[-D-Trp-Lys-Thr(OBzl)-Z1-Tyr-]

SGnc 38 was synthesized (2 mL, 66.8 mg of the Fmoc-Tyr-OH loaded TCP resin), according to GP 3. Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. According to GP 3 Fmoc-Thr(OBzl)-OH, Fmoc-Lys(ivDde)-OH, and Fmoc-D-Trp-OH were coupled consecutively. Subsequent cleavage from the resin (GP 3), cyclization according to GP 4, ivDde deprotection according to GP 5, and purification via RP-HPLC (semipreparative; gradient: 35-50%B in 30 min), yielded the SGnc 38 as a white fluffy powder: ESI-MS: 892.2 [M+K]⁺; 876.5 [M+Na]⁺; 860.9 [M+Li]⁺; 854.4 [M+H]⁺; 796.3 [M-acetone+H]⁺; t_{R}=8.82 min (HPLC-MS, 30-90%B in 15 min).

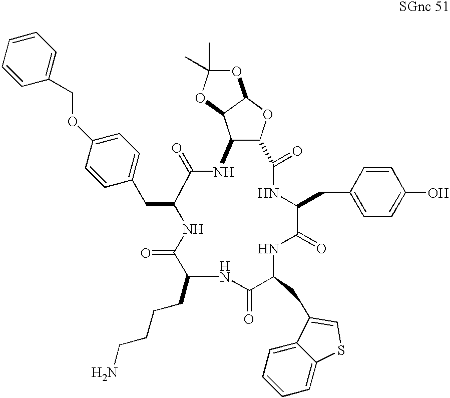

Synthesis of SGnc 51:

SGnc 51 was synthesized (2 mL, 66.8 mg of the Fmoc-Tyr-OH loaded TCP resin), according to GP 3. Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. Subsequent coupling of Fmoc-Tyr(OBzl)-OH (GP 3) was verified by a sample cleavage ESI-MS: 1742.9 [2M−H+Na+K]⁺; 1727.3 [2M−H+2Na]⁺; 1722.2 [2M(1*¹³C)+K]⁺; 1706.3 [2M(1*¹³C)+Na]⁺; 1705.3 [2M+Na]⁺; 1683.2 [2M+H]⁺; 902.4 [M−H+Na+K]⁺; 886.4 [M−H+2Na]⁺; 880.4 [M+K]⁺; 864.5 [M+Na]⁺; 842.3 [M+H]⁺; 784.4 [M-acetone+H]⁺. According to GP 3 Fmoc-Lys(ivDde)-OH, and Fmoc-D-Trp-OH were coupled consecutively. Subsequent cleavage from the resin (GP 3), and cyclization according to GP 4, yielded the cyclic precursor cyclo[-D-Trp-Lys(ivDde)-Tyr(OBzl)-Z1-Tyr-]: ESI-MS: 1177.8 [M+K]⁺; 1161.7 [M+Na]⁺; 1139.7 [M+H]⁺; 1081.7 [M-acetone+H]⁺. ivDde deprotection according to GP 5, and purification via RP-HPLC (semipreparative; gradient: 40-65%B in 30 min), yielded the SGnc 51 as a white fluffy powder: ESI-MS: 1926.5 [2M(1*¹³C)−H+Na+K]⁺; 1903.9 [2M+K]⁺; 1888.9 [2M(1*¹³C)+Na]⁺; 1866.9 [2M(1*¹³C)+H]⁺; 971.8 [M+K]⁺; 955.7 [M+Na]⁺; 933.6 [M+H]⁺; 883.7 [M-acetone+Li]⁺; 875.7 [M-acetone+H]⁺. t_{R}=11.43 min (HPLC-MS, 30-90%B in 15 min).

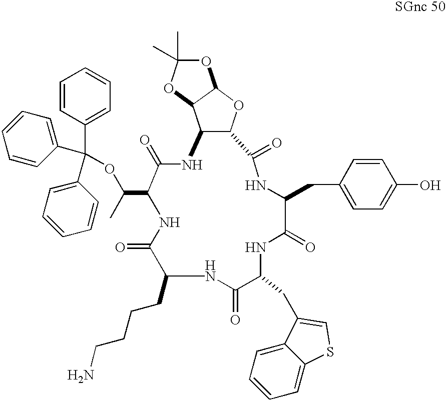

Synthesis of SGnc 50:

SGnc 50 was synthesized (2 mL, 66.8 mg of the Fmoc-Tyr-OH loaded TCP resin), according to GP 3. Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. Subsequent coupling of Fmoc-Thr(OTrt)-OH (GP 3) was verified by a sample cleavage ESI-MS: 1885.3 [2M+K]⁺; 992.6 [M−H+Na+K]⁺; 976.4 [M−H+2Na]⁺; 970.4 [M+K]⁺; 954.4 [M+Na]⁺; 932.6 [M+H]⁺; 734.3 [M−Trt−H+2Na]⁺; 726.0 [M−Trt+K]⁺; 712.4 [M−Trt+Na]⁺; 690.3 [M−Trt+H]⁺; 678.7 [M−Trt-acetone+K]⁺; 663.5 [M−Trt-acetone+Na]⁺; 632.3 [M−Trt-acetone+H]⁺; 243.2 [Trt]⁺. According to GP 3 Fmoc-Lys(ivDde)-OH, and Fmoc-D-Bta-OH were coupled consecutively. Subsequent cleavage from the resin (GP 3), and cyclization according to GP 4, yielded the cyclic precursor cyclo[-D-Bta-Lys(ivDde)-Thr(OTrt)-Z1-Tyr-]: ESI-MS: 1269.0 [M(1*¹³C)+K]⁺; 1251.8 [M+Na]⁺; 1230.6 [M(1*¹³C)+H]⁺; 1009.8 [M−Trt+Na]⁺; 987.6 [M−Trt+H]⁺; 243.2 [Trt]⁺. ivDde deprotection according to GP 5, and purification via RP-HPLC (semipreparative; gradient: 40-65%B in 30 min), yielded the SGnc 50 as a white fluffy powder: ESI-MS: 1061.6 [M+K]⁺; 1045.6 [M+Na]⁺; 1029.8 [M+Li]⁺; 1023.5 [M+H]⁺; 842.6 [M−Trt−H+Na+K]⁺; 828.5 [M−Trt−H+2Na]⁺; 781.5 [M−Trt+H]⁺; 723.5 [M−Trt-acetone+H]⁺, 243.2 [Trt]⁺. t_{R}=12.29 min (HPLC-MS, 30-90%B in 15 min).

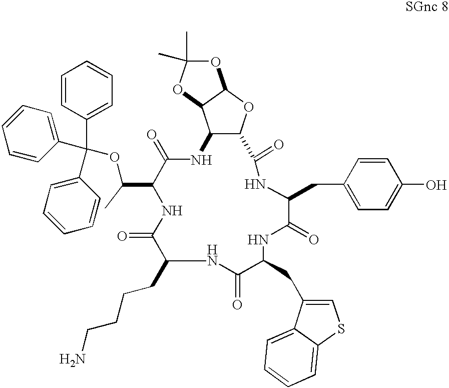

Synthesis of SGnc 8:

SGnc 8 was synthesized (2 mL, 66.8 mg of the Fmoc-Tyr-OH loaded TCP resin), according to GP 3. Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. Subsequent coupling of Fmoc-Thr(OTrt)-OH (GP 3) was verified by a sample cleavage ESI-MS: 1885.3 [2M+K]⁺; 992.6 [M−H+Na+K]⁺; 976.4 [M−H+2Na]⁺; 970.4 [M+K]⁺; 954.4 [M+Na]⁺; 932.6 [M+H]⁺; 734.3 [M−Trt−H+2Na]⁺; 726.0 [M−Trt+K]⁺; 712.4 [M−Trt+Na]⁺; 690.3 [M−Trt+H]⁺; 678.7 [M−Trt-acetone+K]⁺; 663.5 [M−Trt-acetone+Na]⁺; 632.3 [M−Trt-acetone+H]⁺; 243.2 [Trt]⁺. According to GP 3 Fmoc-Lys(ivDde)-OH, and Fmoc-L-Bta-OH were coupled consecutively. Subsequent cleavage from the resin (GP 3), and cyclization according to GP 4, yielded the cyclic precursor cyclo[-Bta-Lys(ivDde)-Thr(OTrt)-Z1-Tyr-]: ESI-MS: 1267.8 [M+K]⁺; 1251.8 [M+Na]⁺; 1229.3 [M+H]⁺; 1009.7 [M-Trt+Na]⁺; 987.6 [M−Trt+H]⁺; 929.7 [M−Trt-acetone+H]⁺; 243.2 [Trt]⁺. ivDde deprotection according to GP 5, and purification via RP-HPLC (semipreparative; gradient: 40-65%B in 30 min), yielded the SGnc 8 as a white fluffy, powder: ESI-MS: 1061.6 [M+K]⁺; 1053.6 [M−H+Li+Na]⁺; 1045.6 [M+Na]⁺; 1029.5 [M+Li]⁺; 1023.5 [M+H]⁺; 842.6 [M−Trt−H+Na+K]⁺; 826.4 [M−Trt−H+2Na]⁺; 781.4 [M−Trt+H]⁺; 723.4 [M−Trt-acetone+H]⁺; 243.2 [Trt]⁺. t_{R}=12.29 min (HPLC-MS, 30-90%B in 15 min).

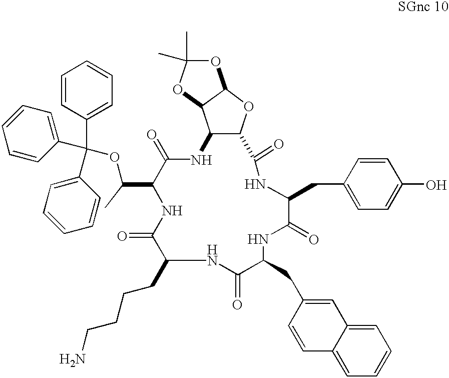

Synthesis of SGnc 10:

SGnc 10 was synthesized (2 mL, 66.8 mg of the Fmoc-Tyr-OH loaded TCP resin), according to GP 3. Coupling of the Fmoc-protected Z1 was verified by a sample cleavage: Some beads were fished out, and the dipeptide Fmoc-Z1-Tyr-OH cleaved from those beads in an Eppendorf cap according to GP 3. ESI-MS of that sample cleavage: 1803.0 [3M+K]⁺; 1786.9 [3M+Na]⁺; 1237.3 [2M−H+Na+K]⁺; 1221.4 [2M−H+2Na]⁺; 1215.3 [2M+K]⁺; 1199.1 [2M+Na]⁺; 633.4 [M−H+2Na]⁺; 627.4 [M+K]⁺; 611.3 [M+Na]⁺; 589.1 [M+H]⁺. Subsequent coupling of Fmoc-Thr(OTrt)-OH (GP 3) was verified by a sample cleavage ESI-MS: 1885.3 [2M+K]⁺; 992.6 [M−H+Na+K]⁺; 976.4 [M−H+2Na]⁺; 970.4 [M+K]⁺; 954.4 [M+Na]⁺; 932.6 [M+H]⁺; 734.3 [M−Trt−H+2Na]⁺; 726.0 [M−Trt+K]⁺; 712.4 [M−Trt+Na]⁺; 690.3 [M−Trt+H]⁺; 678.7 [M−Trt-acetone+K]⁺; 663.5 [M−Trt-acetone+Na]⁺; 632.3 [M−Trt-acetone+H]⁺; 243.2 [Trt]⁺. According to GP 3 Fmoc-Lys(ivDde)-OH, and Fmoc-2-Nal-OH were coupled consecutively. Subsequent cleavage from the resin (GP 3), and cyclization according to GP 4, yielded the cyclic precursor cyclo[-2-Nal-Lys(ivDde)-Thr(OTrt)-Z1-Tyr-]: ESI-MS: 1261.7 [M+K]⁺; 1245.6 [M+Na]⁺; 1230.6 [M(1*¹³C)+Li]⁺; 1224.1 [M(1*¹³C)+H]⁺; 1026.6 [M−Trt−H+2Na]⁺; 1018.7 [M−Trt+K]⁺; 1003.6 [M−Trt+Na]⁺; 981.5 [M−Trt+H]⁺; 923.5 [M−Trt-acetone+H]⁺; 243.2 [Trt]+. ivDde deprotection according to GP 5, and purification via RP-HPLC (semipreparative; gradient: 40-63%B in 30 min), yielded the SGnc 10 as a white fluffy powder: ESI-MS: 1175.3 [M+TFA−H+2Na]⁺; 1169.4 [M+TFA+K]⁺; 1153.3 [M+TFA+Na]⁺; 1056.6 [M(1*¹³C)+K]⁺; 1039.6 [M+Na]⁺; 1017.3 [M+H]⁺; 775.5 [M−Trt+H]⁺; 717.4 [M−Trt-acetone+H]⁺; 243.2 [Trt]⁺. t_{R}=14.46 min (HPLC-MS, 30-70%B in 15 min).

### Example 6

80 mg of a TCP resin loaded with Fmoc-D-Asp-ODmab (i.e. Fmoc-D-Asp bound to the resin through the acid group of the side chain) wherein the loading corresponds to 0.037 mmol/g resin were weighed into a syringe. Before the 1^{st} coupling, the acid function was deprotected 3 times with 3% hydrazine in NMP solution followed by washing with 5% DIPEA in NMP (2×) and NMP (5×). With agitation, the acid was preactivated with a solution consisting of 0.6 equiv. each of HATU, HOAt and 30 equiv. of collidine in 300 μl of NMP for 30 min. with agitation before adding 3 equiv. of 1-(aminomethyl) naphthaline. After 2 hrs., the coupling solution was discarded, the resin washed with NMP (3×) and preactivated once more with 0.6 equiv. of HATU, HOAt and 30 equiv. of collidine in 300 μl of NMP for 30 minutes before adding 3 equiv. of the 1-(aminomethyl) naphthalene. After 2 hours, the coupling solution was discarded and the resin washed 5× with NMP. After that, synthesis was carried out analogously to the synthesis of the above cyclopeptides described in examples 2 to 4.

A few resin beads were taken and treated with a few drops of a 20 vol.-% HFIP in DCM solution in an Eppendorf-Cap for 30 minutes. The amino acid thus separated from the resin:

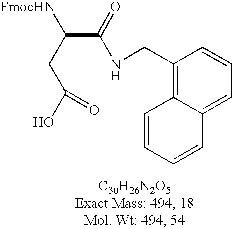

was characterised through an ESI mass spectrum: ESI-MS: 1010.8 [2M+Na]⁺; 989.5 [2M+H]⁺; 517.2 [M+Na]⁺; 495.4 [M+H]⁺.

### Example 7

### Biological Evaluation: Apoptosis-Inducing Effect Both in Multi-Resistant and Non-Resistant Hepatoma Cancer Cell Lines

Rat hepatoma cells were cultivated in a F 12 medium (GibcoBRL), to which 5% of foetal calf serum had been added, in a atmosphere saturated with humidity (>95%) and having a CO₂ content of 8% in air. The cell line named “Klon 2” was isolated by Venetianer et al. (Cytogentc.Cell.Genet 28:280-283, 1980). The cell line 2 (10×80)T1 is a sub-clone of Klon 2 having a moderate multi-drug resistance 8 (Pirity, Hever-Szabo and Venetianer, Cytotechnology 19:207-214, 1996). The degree of resistance of cell line 2 (10×80) was determined by a Niagara blue exclusion test, the cells being exposed to different concentrations of the following cytostatic agents for 72 hrs. The following IC₅₀ values were determined for the cell line: 5.2 for vinblastine, 9.4 for doxorubicine, 11.4 for puromycin, 7.7 for actinomycin D and for colchicine (Pririty et al., Cytotechnology 19:207-214, 1996).

The XTT/PMS Assay (Scuderio et al., Cancer Res. 48:4827-4833, 1988; Roehm et al., J.Immun.Methods 142:257-265, 1991) was utilised to determine the cytotoxicity of the compounds. For this purpose, the viability of the sensitive cell line Klon 2 was tested in comparison with that of the multi-drug resistant cell line Klon 2 (10×80). An identical number of cells was applied to a 96 cell culture plate. After one day, the cells were incubated with different concentrations of the compounds to be tested, compound TT-232 serving as internal control. The cell viability was determined by triple determination for each concentration by means of the XTT/PMS dye test (Scuderio et al., Cancer Res. 48:4827-4833, 1988; Roehm et al., J.Immun.Methods 142:257-265,1991). After an incubation time of 72 hrs. the absorption of treated cells at 450 nm in relation to cells not treated with dye was used as a viability standard. The concentrations of the test compound having 50% viability (IC₅₀) was determined by double determination in two independent experiments.

The following results were obtained:

| | multidrug resistant | drug sensitive |
|---|---|---|
| Activity [μm] | cells | cells |
| c[-Tyr-D-Trp-Lys-Thr(OTrt)-Z1-] | 25 | 31 |
| (TH of Example 2) | | |
| c[-Tyr-Trp-Lys-Thr-Z1-] | 47 | 75 |

These results demonstrate that high activities can be achieved with the compounds according to the invention in cells with multiple drug resistance as well as in cells that do not exhibit such a resistance.

### Example 8

### Biological Evaluation

The Compounds shown below were tested on two cell-lines, A431 (A. T. C. C. reference No. CRL-1555, c.f. American Type Culture Collection, http://phage.atcc.org/cgi-bin/searchengine/longview.cgi?view=ce,663682,CRL-1555&text=a-431, 2001, pp. http://phage.atcc.org/cgi-bin/searchengine/longview.cgi?view-ce,663682,CRL-661555&text=a-663431; http://phage.atcc.org/cgi-bin/searchengine/longview.cgi?view=ce,663682,CRL-661555&text=a-663431) (an epidermoid cancer) and Panc-1 (A.T.C.C. reference No. CRL-1469, c.f. American Type Culture Collection, http://phage.atcc.org/cgi-bin/searchengine/longview.egi?view=ce,609764,CRL-1469&text=panc-1) (a well differentiated pancreatic adenocarcinoma), both of human origin, using the MTT (Carmichael J et al. Cancer Res. 47(4): pp. 936-42, 1987.) and MB (Oliver M H, Harrison N K, Bishop J E, Cole P J, Laurent G J; J Cell Sci 1989 March;92 ( Pt 3):513-8) assays.

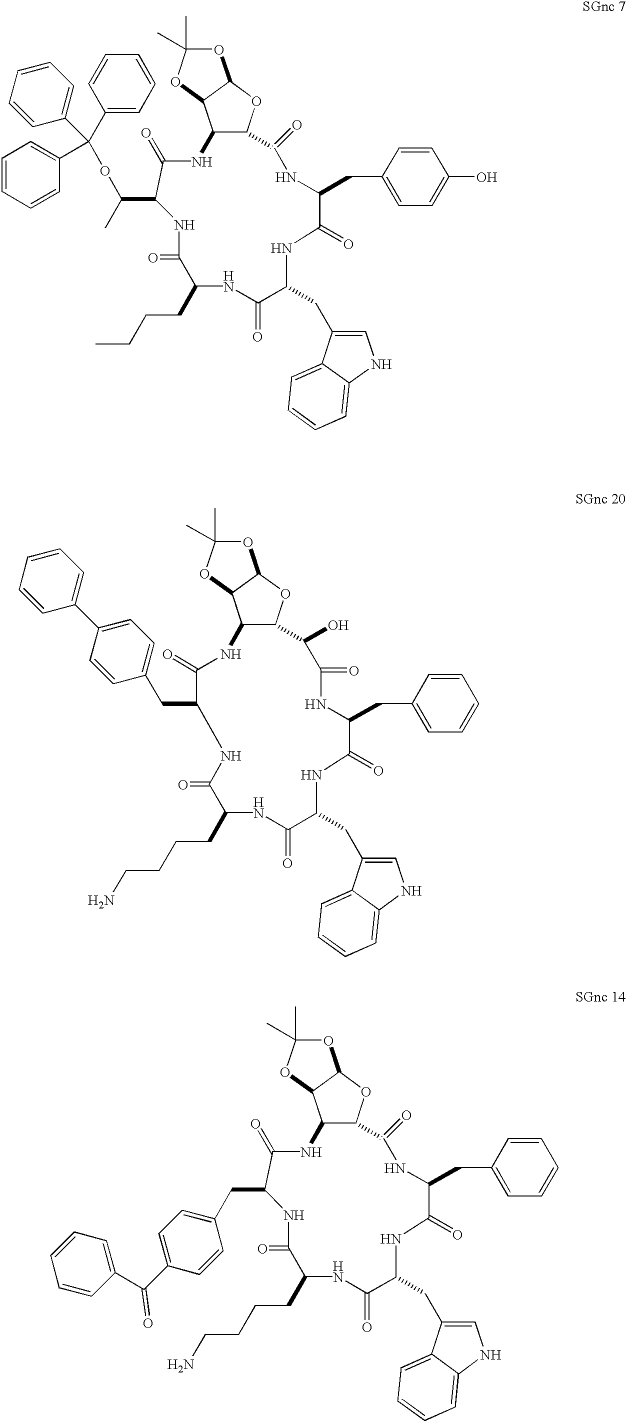

Each compound was tested under 4 conditions: 6 h (to exclude necrosis) and 48 h to see inhibition of proliferation and apoptosis. High ratio between 48/6 h inhibition shows little necrotic, but pronounced apoptotic activity of the tested compound. The results are summarized in Table 1.

TABLE 1
Apoptotic activity of the compounds shown above.

| compound | IC₅₀ [μM]* | Necrosis** |
|---|---|---|
| SGnc 7 | =10 | none |
| SGnc 18 | ˜50 | some |
| SGnc 20 | ˜60 | some |
| SGnc 14 | ˜100 | almost none |
| SGnc 15 | ˜110 | some |
| SGnc 38 | ˜50 | some |
| SGnc 51 | =35 | almost none |
| SGnc 50 | ˜38 | none |
| SGnc 8 | 40^{Panc-1} | some |
| | 50^{A431} | |
| SGnc 10 | 40^{Panc-1} | some |
| | 55^{A431} | |
| *Cell lines used for IC₅₀ determination. A431 (an epidermoid cancer) and Panc-1 (a well differentiated pancreatic adenocarcinoma), both of human origin. Only when the IC₅₀ values were not the same for both cell lines, different IC₅₀ values are given with the respective corresponding cell line noted in the superscript index. For the IC₅₀ determination for both A431 and Panc-1 the MB [Oliver MH, Harrison NK, Bishop JE, Cole PJ, Laurent GJ; J Cell Sci 1989 Mar; 92 | | |
| # (Pt 3):513-8] and the MTT assays were used according to Carmichael J et al. Cancer Res. 47(4): pp. 936-42, 1987. | | |

**The quantity of necrosis detected as number of cell deaths after 6 h of incubation.

### Example 9

### Biological Evaluation: Inhibition of the Mediator Release of Neurolenic Inflammation

Neurogenic inflammation participates in all inflammatory responeses where nociception or pain sensation occurs. The principal mediator of this type of inflammation is Substance P. Classical anti-inflammatory agents as the cyclooxygenase (COX) inhibitors do not inhibit neurogenic inflammation. Stable peptide analogues of somatostatin are potent broad spectrum anti-inflammatory agents which inhibit both the release of Substance P from sensory nerve terminals and also the development of neurogenic inflammation (Helyes, Zs., Pintér, E., Németh, J., Kéri, Gy., Thán,M., Oroszi, G., Horváth, A. and Szolcsányi, J.: Anti-inflammatory effect of synthetic somatostatin analogues in the rat. Br. J. Pharmacol. 134, 1571-1579, 2001, Pintér, E., Helyes, Zs, Németh, J., Pórszász, R., Peth\acute{\acute{o}}, G., Thán, M., Kéri Gy., Horváth A., Jakab B., Szolcsányi, J.: Pharmacological characterization of the somatostatin analogue TT-232: effects on neurogenic and non-neurogenic inflammation and neuropathic hyperalgesia. Naunyn-Schmiedeberg's Arch. Pharmacol. (2002, in press)).

Effect of TG, SGA, TR, and TT-232 on the Release of Substance P in vitro Methods:

After exsanguination the tracheae of 2-2 female Wistar rats were removed and perfused (1 ml min⁻¹) in an organ bath (1.8 ml) at 37° C. for 60 min with oxygenated (95% O₂ and 5% CO₂) Krebs solution of the following composition (in mM): NaCl 119, NaHCO₃ 25, KH₂PO₄ 1.2, MgSO₄ 1.5, KCl 4.7, CaCl₂ 2.5, glucose 11. After stopping the flow the solution was changed 3 times for 8 min (prestimulated—stimulated—poststimulated). Electrical field stimulation (40 V, 0.1 ms, 10 Hz, 120 s) was performed to induce release of sensory neuropeptides from the tissue pieces in the presence or absence of SGTG, SGA, SGTH, or TT-232 (500-500 nM). The fractions were collected in ice-cold tubes and the wet weight of the tracheae were measured. Concentration of SP was determined by specific radioimmunoassay (RIA) methods developed in our laboratory (Németh, J., Oroszi, G., Thán, M., Helyes, Zs., Pintér, E., Farkas, B. and Szolcsányi, J.: Substance P radioimmunoassay for quantitative characterization of sensory neurotransmitter release. Neurobiology, 7, 437-444, 1999) and was expressed as the released amount of peptide per tissue weight.

Results:

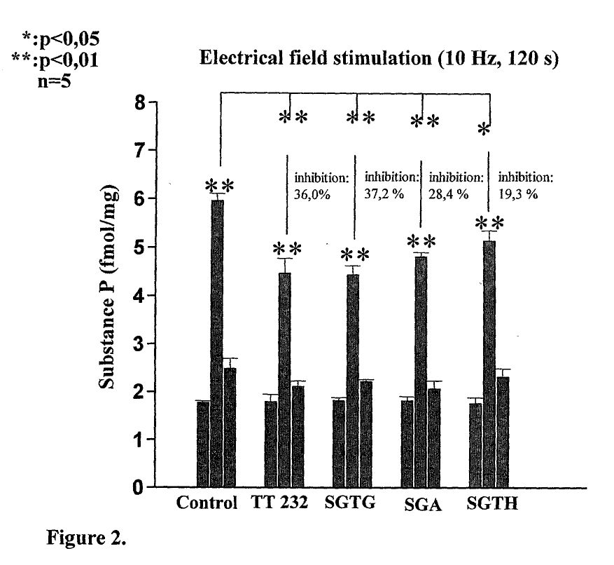

The Results which are summarized in Table 2 below and depicted in FIG. 2 show that Substance P release evoked by electrical stimulation of sensory nerve terminals is inhibited by SGTG, SGA and SGTH to a similar extent as elicited by TT-232.

TABLE 2

| | | | | |
|---|---|---|---|---|
| | TT 232 | SGTG | SGA | SGTH |
| Control | 500 nmol | 500 nmol | 500 nmol | 500 nmol |

| pre | | post | pre | | post | pre | | post | pre | | post | pre | | post |
|---|---|---|---|---|---|---|---|---|---|---|---|---|---|---|
| stim. | stim. | stim. | stim. | stim. | stim. | stim. | stim. | stim. | stim. | stim. | stim. | stim. | stim. | stim. |
| 1.77 ± | 5.96 ± | 2.48 ± | 1.79 ± | 4.47 ± | 2.11 ± | 1.81 ± | 4.44 ± | 2.21 ± | 1.81 ± | 4.81 ± | 2.07 ± | 1.76 ± | 5.14 ± | 2.31 ± |
| 0.04 | 0.15 | 0.22 | 0.15 | 0.30 | 0.12 | 0.09 | 0.18 | 0.05 | 0.09 | 0.09 | 0.16 | 0.12 | 0.21 | 0.18 |

| | | | |
|---|---|---|---|
| inhibition | inhibition | inhibition | inhibition |
| 36.0% | 37.2% | 28.4% | 19.3% |

1. A peptide selected from the general formulae 1, 2, 3, 4, 5, 6, and pharmaceutically acceptable salts thereof:
y¹-Aₙ-B—C-Dₘ-Z-y² (1)
y¹-Z-Aₙ-B—C-Dₘ-y² (2)
y¹-Dₘ-Z-Aₙ-B—C-y² (3)
—C-Dₘ-Z-Aₙ-B-y² (4)
y¹-B—C-Dₘ-Z-Aₙ-y² (5) 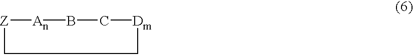
wherein Z is a radical of the general formula (7) 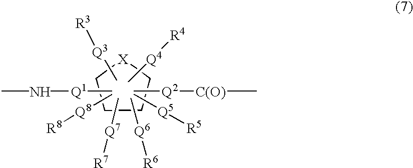
wherein the substituents Q¹, Q², Q³, Q⁴, Q⁵, Q⁶, Q⁷, Q⁸, R³, R⁴, R⁵, R⁶, R⁷, R⁸ and X have the following meaning:
X is selected from O, S, Se, NR⁹, PR⁹ and CR⁹R¹⁰, wherein R⁹, R¹⁰ are independently selected from H, OH, SH, F, Cl, Br, I, alkyl, alkenyl, alkinyl, aryl, alkylaryl, arylalkyl, alkoxy, alkenyloxy, aryloxy, thioalkyl, thioaryl, selenoalkyl, selenoaryl, which may optionally be substituted with F, OH, SH, SeH, an amino group, an oxo group or a carboxy group;
Q¹ and Q² are independently selected from a single bond, CH₂, CH(OH), CH(OR¹), CHR¹ and CR¹R²;
wherein R¹ and R² are independently selected from alkyl, alkenyl, aryl, arylalkyl, alkylaryl, which may optionally be substituted with F, OH, an amino group or a carboxy group;
Q³ to Q⁸ are independently selected from a single bond, O, S, Se, N₂, NR⁹, PO₃;
R³ to R⁸ are independently selected from the group consisting of H, OH, SH, N₃, CN, NC, SCN, F, Cl, Br, I, SO₃, NO₂, PR¹¹R¹², COOR¹¹, alkyl, alkenyl, alkinyl, aryl, alkylaryl, arylalkyl, alkanoyl, alkenoyl, alkinoyl, aroyl, arylalkanoyl, alkylaroyl, which may optionally be substituted with F, OH, SH, SeH, an amino group, an oxo group or a carboxy group;
wherein R¹¹ and R¹² are independently selected from H, OH, SH, F, Cl, Br, I, CN, NC, SCN, alkyl, alkenyl, alkinyl, aryl, alkylaryl, arylalkyl, alkoxy, alkenyloxy, aryloxy, thioalkyl, thioalkenyl, thioaryl, selenoalkyl, selenoalkenyl, selenoaryl, amidoalkyl, amidoalkenyl, amidoalkinyl, arylalkanoyloxy, alkylaroyloxy, arylalkoxy, alkylaryloxy, which may optionally be substituted with F, OH, SH, SeH, an amino group, an oxo group or a carboxy group;
wherein two substituents Rⁱ and R^{j}, with i, j=3 to 8, may optionally be linked, forming a 5- or 6-membered ring, wherein optionally one or more of the ring atoms are independently substituted with one or more groups selected from alkyl, alkenyl and aryl;
wherein the radicals, A, B, C and D have the following meaning:
A is an α-, β- or γ-amino carboxylic acid radical having an aromatic side chain or an aliphatic side chain;
B is an α-, β- or γ-amino carboxylic acid radical having an aromatic side chain;
C is an α-, β- or γ-amino carboxylic acid radical having a basic side chain or an aliphatic side chain;
D is an α-, β- or γ-amino carboxylic acid radical which does not have acidic groups or basic groups in the side chain;
wherein y¹ is linked to the amino group of the corresponding amino carboxylic acid and is selected from H, CH₃(CH₂)ᵣCO, with r=0 to 6, butoxy carbonyl and 9-fluorenyl methyoxy carbonyl;
wherein y² is linked to the carboxy group of the corresponding amino acid and is selected from H, NH₂, alkoxy, aryloxy, alkyl, aryl, alkenyl, alkinyl, F, Cl, Br, I, CN, NC, SCN, thioalkyl, thioaryl;
wherein n and m represent integers selected from 0 and 1 such that m+n is 1 or 2;
and the groups A, B, C, D and Z linked to each other via a peptide linkage each.

2. A peptide according to claim 1 wherein X is an oxygen atom.

3. A peptide according to one or more of the claims 1 and 2 wherein the substituents -Qⁱ-Rⁱ, with i=3 to 8, are selected in such a manner that each of the ring atoms in formula (7) except X bears a hydrogen atom and a substituent other than hydrogen.

4. A peptide according to one or more of the claims 1 to 3 wherein the substituents -Q¹-NH— and -Q²-C(O)— are linked to adjacent carbon atoms of the ring in formula (7).

5. A peptide according to one or more of the claims 1 to 4 wherein Q² represents the group CH(OH).

6. A peptide according to one or more of the claims 1 to 5 wherein the substituents -Qⁱ-Rⁱ, with i=3 to 8, are selected from H, alkyl, alkenyl, aryl, arylalkyl, alkylaryl, alkoxy, aryloxy, aroyloxy und alkanoyloxy.

7. A peptide according to one or more of the claims 1 to 6 wherein two of the substituents -Qⁱ-Rⁱ, with i=3 bis 8, jointly form an akyl ketal, an aryl ketal, an alkylaryl ketal, an alkyl acetal or an aryl acetal.

8. A peptide according to claim 7, wherein Z is

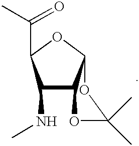

9. A peptide according to one or more of the claims 1 to 8 wherein the side chain of the amino carboxylic acid radical A is an C₁-C₁₀ alkyl group.

10. A peptide according to claim 9 wherein A is a valine radical.

11. A peptide according to one or more of the claims 1 to 8 wherein the side chain of the amino carboxylic acid radical A is an C₆-C₁₄ aryl group which may optionally be substituted with OH or I and wherein a carbon atom may optionally be isosterically replaced by nitrogen or sulfur.

12. A peptide according to one or more of the claims 1 to 8 wherein the side chain of the amino carboxylic acid radical A is a C₁-C₄ Alkyl-C₆-C₁₄ aryl group the aryl group of which may optionally be substituted with OH or I and wherein a carbon atom may optionally be isosterically replaced by nitrogen or sulfur.

13. A peptide according to claim 12 wherein the aminocarboxylic acid radical A may be a phenyl alanine radical or a tyrosine radical.

14. A peptide according to one or more of the claims 1 to 13 wherein the side chain of the amino carboxylic acid racidal B is a C₆-C₁₄ aryl group which may optionally be substituted with OH or I and wherein a carbon atom may optionally be isosterically replaced by nitrogen or sulfur.

15. A peptide according to one or more of the claims 1 to 13 wherein the side chain of the amino carboxylic acid B is a C₁-C₄ alkyl-C₆-C₁₄ aryl group which may optionally be substituted with OH or I and wherein a carbon atom may optionally be isosterically replaced by nitrogen or sulfur.

16. A peptide according to claim 15 wherein the amino carboxylic acid radical B is selected from I-naphthyl alanine, 2-naphthyl alanine, tryptophan und 3-benzothienyl alanine, wherein the amino carboxylic acid radical B may be in the L- or D-configuration.

17. A peptide according to one or more of the claims 1 to 16 wherein the side chain of the amino carboxylic acid radical C is a C₁-C₁₀ alkyl group which may be substituted with one or more of the groups selected from amino, acetyl, trifluoroacetyl and alkyl amide groups.

18. A peptide according to claim 17 wherein the side chain of the amino carboxylic acid radical C is a C₃-C₅ alkyl group.

19. A peptide according to claim 18, wherein the side chain of the amino carboxylic acid radical C is norleucine.

20. A peptide according to claim 17, wherein the side chain of the amino carboxylic acid radical C is a C₃-C₅ amino alkyl group.

21. A peptide according to claim 20 wherein the the amino carboxylic acid radical C is lysine.

22. A peptide according to one or more of the claims 1 to 21 wherein the side chain of the amino carboxylic acid radical D is a C₆-C₁₄ aryl group which may optionally be substituted with OH or I or which is linked to a further aryl group via an ether group and wherein a carbon atom may optionally be isosterically replaced by nitrogen or sulfur.

23. A peptide according to one or more of the claims 1 to 21 wherein the side chain of the amino carboxylic acid radical D is a C₁-C₄ alkyl-C₆-C₁₄ aryl group which may optionally be substituted with OH or I and wherein a carbon atom may optionally be isosterically replaced by nitrogen or sulfur.

24. A peptide according to one or more of the claims 1 to 21 wherein the side chain of the amino carboxylic acid radical D is a C₁-C₆ alkyl group which may optionally be substituted with one or more of the groups selected from OH, C₁-C₁₀ alkoxy, C₆-C₂₀-aryl-C₁-C₄-alkoxy, and C₆-C₂₀ aryloxy.

25. A peptide according to claim 24 wherein the amino carboxylic acid radical D is the trityl ether of L-threonine, the benzyl ether of L-threonine or the benzyl ether of L-tyrosine.

26. A peptide according to claim 25 wherein the side chain of the amino carboxylic acid radical D is the trityl ether of L-threonine.

27. A peptide according to claim 25 or 26 wherein the amino carboxylic acid radical A is L-tyrosine, which may optionally be substituted with ¹²⁵I, or L-phenyl alanine.

28. A peptide according to one or more of the claims 25 to 27 wherein the amino carboxylic acid radical B is D- or L-tryptophan.

29. A peptide according to one or more of the claims 25 to 27 wherein the amino carboxylic acid radical B is D- or L-benzothienylalanin.

30. A peptide according to one or more of the claims 25 to 29 wherein the amino carboxylic acid radical C is L-lysine or L-norleucine.

31. A peptide according to one or more of claims 1 to 21, wherein the peptide is selected from the group of tetrapeptides consisting of cyclo[-Phe-Trp-Lys-Z-], cyclo[Phe-D-Trp-Lys-Z-], cyclo[-Phe-Trp-Nle-Z-], cyclo[-Phe-D-Trp-Nle-Z-], cyclo[-Tyr-Trp-Lys-Z-], cyclo[-Tyr-D-Trp-Lys-Z-], cyclo[-Tyr-Trp-Nle-Z-], cyclo[-Tyr-D-Trp-Nle-Z-], cyclo[-Phe-Bta-Lys-Z-], cyclo[-Phe-D-Bta-Lys-Z-], cyclo[-Phe-Bta-Nle-Z-], cyclo[-Phe-D-Bta-Nle-Z-], cyclo[-Tyr-Bta-Lys-Z-], cyclo[-Tyr-D-Bta-Lys-Z-] and cyclo[-Tyr-Bta-Nle-Z-].

32. A compound derived from a peptide according to one or more of the claims 1 to 31, wherein the peptide is linked to one or more radionuclides suitable for radioscintigraphy or positron emission tomography, via a suitable linker and/or bifunctional chelating agent.

33. A compound according to claim 32, wherein the linker and/or bifunctional chelating agent is derived from a compound selected from EDTA, DFO, DTPA, DOTA, TETA, DADS and short peptides having 2 to 4 amino acids selected from Lys, Gly and Cys.

34. A compound according to the claims 32 or 33, werein each of the radionuclides is selected from ^{99m}Tc and ¹¹¹In, ⁶⁷Ga, ⁶⁸Ga, ⁸⁶Y, ⁹⁰Y and ⁶⁴Cu.

35. A pharmaceutical composition comprising the peptide according to one or more of the claims 1 to 31 and optionally pharmaceutically acceptable excipients and carriers.

36. A pharmaceutical composition according to claim 35 for the treatment of tumours and/or neurological and/or inflammatory disorders and/or pain.

37. A pharmaceutical composition according to claim 36 wherein the tumour is a tumour of the pituitary gland, a mamma carcinoma, glucagonoma, renal carcinoma, prostate carcinoma, meningioma, glioma, pancreas tumour, insulinoma, melanoma or liver tumour.

38. A composition to diagnose tumours by means of positron-emission tomography or scintigraphy comprising a peptide according to one or more of the claims 1 to 31 or a compound according to one or more of claims 32 to 34, wherein the peptide or compound contains one or more radioactive isotopes.

39. A composition according to claim 38 wherein the tumour is a tumour of the pituitary gland, a mamma carcinoma, glucagonoma, renal carcinoma, prostate carcinoma, meningioma, glioma, pancreas tumour, insulinoma, melanoma or liver tumour.

40. The use of the peptide according to one or more of the claims 1 to 31 for the treatment of tumours, neurological disorders and neurological inflammations.

41. The use of the peptide according to the claims 1 to 31 or of the compound according to the claims 32 to 34, wherein the peptide or the compound contains one or more radioactive isotopes, for the diagnosis of tumours by means of positron-emission tomography.

42. The use according to claim 40 or 41 wherein the tumour is a tumour of the * pituitary gland, a mamma carcinoma, glucagonoma, renal carcinoma, prostate carcinoma, meningioma, glioma, pancreas tumour, insulinoma, melanoma or liver tumour.

43. The use according to one or more of the claims 40 to 41 wherein the tumour is resistant against cytostatic agents (multidrug resistant).

44. The use according to one or more of claims 40 to 43 in combination with the use of cytostatic agents.

45. The use according to one or more of claims 40 to 43 wherein the tumor is resistant against one or more other chemotherapeutic agents.

46. The use according to claim 45, wherein the tumor is resistant against one or more other somatostatin derivatives.

47. The use according to claim 46, wherein the tumor is resistant to octreotide.

48. A process for preparing the peptide according to one or more of the claims 1 to 31 using solid-phase peptide synthesis methods and/or by synthesis in solution.
